(19) United States
(12) Patent Application Publication
(10) Pub. No.: US 2023/0250173 A1
(43) Pub. Date: Aug. 10, 2023

(54) BIOMARKERS FOR PD-1 AXIS BINDING ANTAGONIST THERAPY

(71) Applicant: Pfizer Inc., New York, NY (US)

(72) Inventors: Keith Anthony Ching, San Diego, CA (US); Craig Davis, San Diego, CA (US); Xinmeng Mu, San Diego, CA (US); Shobha Potluri, Foster City, CA (US); Yan Qu, Half Moon Bay, CA (US); Shahram Salek-Ardakani, San Diego, CA (US); Graham Thomas, San Diego, CA (US); Ji Wen, San Diego, CA (US)

(21) Appl. No.: 18/002,942

(22) PCT Filed: Jun. 29, 2021
(86) PCT No.: PCT/IB2021/055799

§ 371 Date: Dec. 22, 2022

Related U.S. Application Data
(60) Provisional application No. 63/193,306, filed on May 26, 2021.
(60) Provisional application No. 62/706,912, filed on Sep. 17, 2020.
(60) Provisional application No. 62/706,712, filed on Sep. 4, 2020.
(60) Provisional application No. 63/046,957, filed on Jul. 1, 2020.

(51) Int. Cl. C07K 16/28; C12Q 1/6886; A61P 35/00; A61P 13/02

(52) U.S. Cl. CPC C07K 16/2818; C12Q 1/6886; A61P 35/00; A61P 13/02; C12Q 2600/106; C12Q 2600/158

(57) ABSTRACT

The present disclosure describes therapies comprising a PD-1 axis binding antagonist, wherein a sample from the patient has been pre-determined to have certain biomarkers.

### FIELD

The present invention relates to PD-1 axis binding antagonists to treat cancer in patients in which a sample from the patient has one or more of certain gene expression profiles, gene mutations, and/or other biomarkers for use with PD-1 axis antagonists, and related methods and compositions.

### BACKGROUND

The programmed death 1 (PD-1) receptor and PD-1 ligands 1 and 2 (PD-L1 and PD-L2, respectively), play integral roles in immune regulation. Expressed on activated T cells, PD-1 is activated by PD-L1 (also known as B7-H1) and PD-L2 expressed by stromal cells, tumor cells, or both, initiating T-cell death and localized immune suppression (Dong et al., Nat Med 1999; 5:1365-69; Freeman et al. J Exp Med 2000; 192:1027-34), potentially providing an immune-tolerant environment for tumor development and growth. Conversely, inhibition of this interaction, by a PD-1 axis binding antagonist, can enhance local T-cell responses and mediate antitumor activity (Iwai Y, et al. Proc Natl Acad Sci USA 2002; 99:12293-97). Several PD-1 axis binding antagonists, including the PD-1 antibodies nivolumab (Opdivo), pembrolizumab (Keytruda) and PD-L1 antibodies avelumab (Bavencio), durvalumab (Imfinzi), and atezolizumab (Tecentriq) were approved by the U.S. Food and Drug Administration (FDA) for the treatment of cancer in recent years.

Despite of all the new cancer therapies in the recent years, there remains a need of improved therapies for the treatment of cancers. Furthermore, there is a need for improved methods and compositions for identifying patients that will respond to treatment with PD-1 axis binding antagonists.

### SUMMARY

Provided herein are biomarkers for use with PD-1 axis binding antagonist therapy, PD-1 axis binding antagonists for use with samples that have been predetermined to have one or more biomarkers of interest, and related compositions and methods.

In some embodiments, provided herein is method of treating a patient having a cancer, comprising administering to the patient a therapeutically effective amount of a PD-1 axis binding antagonist, wherein the expression level of at least one gene selected from the group consisting of Irf1, Stat1, and Gbp2 in a sample obtained from the patient has been determined to be increased as compared to a reference level prior to treatment. Optionally, the expression level of at least two or all three of the genes selected from the group consisting of Irf1, Stat1, and Gbp2 in the sample obtained from the patient has been determined to be increased as compared to a reference level. Optionally further, the expression level of at least one gene selected from the group consisting of Tap1, Psmb9, Ccl5 and Cd38 in the sample obtained from the patient has been determined to be increased as compared to a reference level. Optionally further, the expression level of at least two genes selected from the group consisting of Tap1, Psmb9, Ccl5 and Cd38 in the sample obtained from the patient has been determined to be increased as compared to a reference level. Optionally further, the expression level of at least one gene selected from the group consisting of Slamf8, Calhm6, Cd40, Cxcl9, PD-L1, Nos2, M6pr, Cd74, MHCII, and Ly6l in the sample obtained from the patient has been determined to be increased as compared to a reference level. Optionally further, the expression level of at least one gene selected from the group consisting of Cxcl10, Tor3a, Xaf1, Nlrp3, Ptgs2, Bhlhe40, Socs3, Socs1, Irf8, Cd86, Cd273, Gbp3, Plekho1, Lap3, Tnfaip2, Psme2, Atox1, Ube2I6, Scimp, Nfkbie, Tapbp, Trafd1, Rnf19b, Pomp, Dram1, and Syngr2 in the sample obtained from the patient has been determined to be increased as compared to a reference level. Optionally, the sample comprises leukocytes. Optionally, the sample further comprises at least one cell type selected from the group consisting of CD45+ cells, myeloid cells, and tumor-associated macrophages. Optionally, the sample consists of leukocytes and at least one cell type selected from the group consisting of CD45+ cells, myeloid cells, and tumor-associated macrophages. Optionally, the sample comprises or consists of leukocytes, CD45+ cells, myeloid cells, or tumor-associated macrophages.

In some embodiments, provided herein is a method of treating a patient having a cancer, comprising administering to the patient a therapeutically effective amount of a PD-1 axis binding antagonist, wherein the expression level of at least two, three, four, five, six, or all seven genes selected from the group consisting of Irf1, Stat1, Gbp2, Tap1, Psmb9, Ccl5 and Cd38 in a sample obtained from the patient has been determined to be increased as compared to a reference level prior to treatment. Optionally, the sample comprises leukocytes. Optionally, the sample further comprises at least one cell type selected from the group consisting of CD45+ cells, myeloid cells, and tumor-associated macrophages. Optionally, the sample consists of leukocytes and at least one cell type selected from the group consisting of CD45+ cells, myeloid cells, and tumor-associated macrophages. Optionally, the sample comprises or consists of leukocytes, CD45+ cells, myeloid cells, or tumor-associated macrophages.

In some embodiments, provided herein is a method of treating a patient having a cancer, comprising administering to the patient a therapeutically effective amount of a PD-1 axis binding antagonist, wherein the expression level of at least one gene selected from the group consisting of Slamf8 and Calhm6 in a sample obtained from the patient has been determined to be increased as compared to a reference level prior to treatment. Optionally, the expression level of both Slamf8 and Calhm6 in the sample obtained from the patient has been determined to be increased as compared to a reference level. Optionally further, the expression level of at least one gene selected from the group consisting of Cd40, Cxcl9, and PD-L1 in the sample obtained from the patient has been determined to be increased as compared to a reference level. Optionally further, the expression level of at least two genes selected from the group consisting of Cd40, Cxcl9, and PD-L1 in the sample obtained from the patient has been determined to be increased as compared to a reference level. Optionally further, the expression level of at least one gene selected from the group consisting of Nos2, Ccl5, M6pr, Cd38, Cd74, MHCII, Stat1, and Ly6l in the sample obtained from the patient has been determined to be increased as compared to a reference level. Optionally further, the expression level of at least one gene selected from the group consisting of Cxcl10, Tor3a, Xaf1, Nlrp3, Ptgs2, Bhlhe40, Socs3, Socs1, Irf8, Cd86, Cd273, Gbp3, Plekho1, Gbp2, Lap3, Tap1, Irf1, Tnfaip2, Psme2, Atox1, Ube2I6, Scimp, Nfkbie, Psmb9, Tapbp, Trafd1, Rnf19b, Pomp, Dram1, and Syngr2 in the sample obtained from the patient has been determined to be increased as compared to a reference level. Optionally, the sample comprises leukocytes. Optionally, the sample further comprises at least one cell type selected from the group consisting of CD45+ cells, myeloid cells, and tumor-associated macrophages. Optionally, the sample consists of leukocytes and at least one cell type selected from the group consisting of CD45+ cells, myeloid cells, and tumor-associated macrophages. Optionally, the sample comprises or consists of leukocytes, CD45+ cells, myeloid cells, or tumor-associated macrophages.

In some embodiments, provided herein is a method of treating a patient having a cancer, comprising administering to the patient a therapeutically effective amount of a PD-1 axis binding antagonist, wherein the expression level of at least two, three, four, five, six, seven, eight, nine, ten, eleven, twelve, or all thirteen genes selected from the group consisting of Slamf8, Calhm6, Cd40, Cxcl9, Nos2, Ccl5, M6pr, Cd38, Cd74, MHCII, Stat1, and Ly6l in a sample obtained from the patient has been determined to be increased as compared to a reference level prior to treatment. Optionally, the sample comprises leukocytes. Optionally, the sample further comprises at least one cell type selected from the group consisting of CD45+ cells, myeloid cells, and tumor-associated macrophages. Optionally, the sample consists of leukocytes and at least one cell type selected from the group consisting of CD45+ cells, myeloid cells, and tumor-associated macrophages. Optionally, the sample comprises or consists of leukocytes, CD45+ cells, myeloid cells, or tumor-associated macrophages.

In some embodiments, provided herein is a method of treating a patient having a cancer, comprising administering to the patient a therapeutically effective amount of a PD-1 axis binding antagonist, wherein the expression level of at least one gene selected from the group consisting of Slamf8, Calhm6, Cd40, Cxcl9, and PD-L1 in tumor-associated myeloid cells obtained from the patient has been determined to be increased as compared to a reference level prior to treatment. Optionally, the expression level of at least two, three, four, or all five of the genes selected from the group consisting of Slamf8, Calhm6, Cd40, Cxcl9, and PD-L1 in tumor-associated myeloid cells obtained from the patient has been determined to be increased as compared to a reference level. Optionally further, the expression level of at least one gene selected from the group consisting of Nos2, Ccl5, M6pr, Cd38, Cd74, MHCII, Stat1, and Ly6l in tumor-associated myeloid cells obtained from the patient has been determined to be increased as compared to a reference level. Optionally further, the expression level of at least one gene selected from the group consisting of Cxcl10, Tor3a, Xaf1, Nlrp3, Ptgs2, Bhlhe40, Socs3, Socs1, Irf8, Cd86, Cd273, Gbp3, Plekho1, Gbp2, Lap3, Tap1, Irf1, Tnfaip2, Psme2, Atox1, Ube2I6, Scimp, Nfkbie, Psmb9, Tapbp, Trafd1, Rnf19b, Pomp, Dram1, and Syngr2 in tumor-associated myeloid cells obtained from the patient has been determined to be increased as compared to a reference level.

In some embodiments, provided herein is a method of treating a patient having a cancer, comprising administering to the patient a therapeutically effective amount of a PD-1 axis binding antagonist, wherein the expression level of the gene Cxcl9, and at least one of the genes selected from the group consisting of Irf1, Stat1, Gbp2, Slamf8, and Calhm6, in a sample obtained from the patient has been determined to be increased as compared to a reference level prior to treatment. Optionally, the sample comprises leukocytes. Optionally, the sample further comprises at least one cell type selected from the group consisting of CD45+ cells, myeloid cells, and tumor-associated macrophages. Optionally, the sample consists of leukocytes and at least one cell type selected from the group consisting of CD45+ cells, myeloid cells, and tumor-associated macrophages. Optionally, the sample comprises or consists of leukocytes, CD45+ cells, myeloid cells, or tumor-associated macrophages.

In some embodiments, provided herein is a method of identifying a patient having a cancer who may benefit from a treatment comprising a therapeutically effective amount of a PD-1 axis binding antagonist, the method comprising determining an expression level of at least one, two, or all three genes selected from the group consisting of Irf1, Stat1, and Gbp2 in a sample obtained from the patient, wherein an increased expression level of the at least one, two, or all three genes in the sample as compared to a reference level identifies the patient as one who has an increased likelihood of benefiting from a treatment comprising a therapeutically effective amount of a PD-1 axis binding antagonist.

In some embodiments, provided herein is a method of predicting responsiveness of a patient having a cancer to a treatment comprising a therapeutically effective amount of a PD-1 axis binding antagonist, the method comprising determining an expression level of at least one, two, or all three genes selected from the group consisting of Irf1, Stat1, and Gbp2 in a sample obtained from the patient, wherein an increased expression level of the at least one, two, or all three genes in the sample as compared to a reference level indicates that the patient has an increased likelihood of benefiting from a treatment comprising a therapeutically effective amount of a PD-1 axis binding antagonist.

In some embodiments, provided herein is a medicament comprising a PD-1 axis binding antagonist for use in treating a cancer in a patient, wherein a sample from the patient is pre-determined to have at least one of and optionally two, three, four, five or all of the following characteristics: (i) it has an increased expression level of at least one, two, or all three of the genes Irf1, Stat1, and Gbp2 as compared to a reference level; (ii) it has an increased expression level of at least 1, 2, 3, 4, 5, 6 or all 7 genes selected from the group consisting of Irf1, Stat1, Gbp2, Tap1, Psmb9, Ccl5 and Cd38 as compared to a reference level; (iii) it has an increased expression level of at least 1, 2, 3, 4, 5, 6, 7, 8, 10, 15, or all 18 genes selected from the group consisting of Irf1, Stat1, Gbp2, Tap1, Psmb9, Ccl5, Cd38, Slamf8, Calhm6, Cd40, Cxcl9, PD-L1, Nos2, M6pr, Cd74, MHCII, and Ly6l as compared to a reference level; (iv) it has an increased expression level of at least 1, 2, 3, 4, 5, 6, 7, 8, 10, 15, 20, or 25 genes selected from the group consisting of Irf1, Stat1, Gbp2, Tap1, Psmb9, Ccl5, Cd38, Slamf8, Calhm6, Cd40, Cxcl9, PD-L1, Nos2, M6pr, Cd74, MHCII, Ly6I, Cxcl10, Tor3a, Xaf1, Nlrp3, Ptgs2, Bhlhe40, Socs3, Socs1, Irf8, Cd86, Cd273, Gbp3, Plekho1, Lap3, Tnfaip2, Psme2, Atox1, Ube2I6, Scimp, Nfkbie, Tapbp, Trafd1, Rnf19b, Pomp, Dram1, and Syngr2 i as compared to a reference level; (v) the sample comprises or consists of leukocytes, CD45+ cells, myeloid cells, or tumor-associated macrophages; (vi) it has an increased expression level of one or both of the genes Slamf8 and Calhm6 as compared to a reference level.

In some embodiments, provided herein is a PD-1 axis binding antagonist for use in a method of treating a cancer in a patient, wherein the method comprises: (a) determining whether a sample from the patient has at least one and optionally two, three, four, five or all of the following characteristics: (i) it has an increased expression level of at least one, two, or all three of the genes Irf1, Stat1, and Gbp2 as compared to a reference level; (ii) it has an increased expression level of at least 1, 2, 3, 4, 5, 6 or all 7 genes selected from the group consisting of Irf1, Stat1, Gbp2, Tap1, Psmb9, Ccl5 and Cd38 as compared to a reference level; (iii) it has an increased expression level of at least 1, 2, 3, 4, 5, 6, 7, 8, 10, 15, or all 18 genes selected from the group consisting of Irf1, Stat1, Gbp2, Tap1, Psmb9, Ccl5, Cd38, Slamf8, Calhm6, Cd40, Cxcl9, PD-L1, Nos2, M6pr, Cd74, MHCII, and Ly6l as compared to a reference level; (iv) it has an increased expression level of at least 1, 2, 3, 4, 5, 6, 7, 8, 10, 15, 20, or 25 genes selected from the group consisting of Irf1, Stat1, Gbp2, Tap1, Psmb9, Ccl5, Cd38, Slamf8, Calhm6, Cd40, Cxcl9, PD-L1, Nos2, M6pr, Cd74, MHCII, Ly6I, Cxcl10, Tor3a, Xaf1, Nlrp3, Ptgs2, Bhlhe40, Socs3, Socs1, Irf8, Cd86, Cd273, Gbp3, Plekho1, Lap3, Tnfaip2, Psme2, Atox1, Ube2I6, Scimp, Nfkbie, Tapbp, Trafd1, Rnf19b, Pomp, Dram1, and Syngr2 i as compared to a reference level; (v) the sample comprises or consists of leukocytes, CD45+ cells, myeloid cells, or tumor-associated macrophages; (vi) it has an increased expression level of one or both of the genes Slamf8 and Calhm6 as compared to a reference level, and (b) if the sample has said characteristic(s), administering to the patient an effective amount of the PD-1 axis binding antagonist.

Use of a PD-1 axis binding antagonist for the manufacture of a medicament for the treatment of cancer in a patient, wherein a sample from the patient is pre-determined to have at least one of and optionally two, three, four, five, or all of the following characteristics: (i) it has an increased expression level of at least one, two, or all three of the genes Irf1, Stat1, and Gbp2 as compared to a reference level; (ii) it has an increased expression level of at least 1, 2, 3, 4, 5, 6 or all 7 genes selected from the group consisting of Irf1, Stat1, Gbp2, Tap1, Psmb9, Ccl5 and Cd38 as compared to a reference level; (iii) it has an increased expression level of at least 1, 2, 3, 4, 5, 6, 7, 8, 10, 15, or all 18 genes selected from the group consisting of Irf1, Stat1, Gbp2, Tap1, Psmb9, Ccl5, Cd38, Slamf8, Calhm6, Cd40, Cxcl9, PD-L1, Nos2, M6pr, Cd74, MHCII, and Ly6l as compared to a reference level; (iv) it has an increased expression level of at least 1, 2, 3, 4, 5, 6, 7, 8, 10, 15, 20, or 25 genes selected from the group consisting of Irf1, Stat1, Gbp2, Tap1, Psmb9, Ccl5, Cd38, Slamf8, Calhm6, Cd40, Cxcl9, PD-L1, Nos2, M6pr, Cd74, MHCII, Ly6I, Cxcl10, Tor3a, Xaf1, Nlrp3, Ptgs2, Bhlhe40, Socs3, Socs1, Irf8, Cd86, Cd273, Gbp3, Plekho1, Lap3, Tnfaip2, Psme2, Atox1, Ube2I6, Scimp, Nfkbie, Tapbp, Trafd1, Rnf19b, Pomp, Dram1, and Syngr2 i as compared to a reference level; (v) the sample comprises or consists of leukocytes, CD45+ cells, myeloid cells, or tumor-associated macrophages; (vi) it has an increased expression level of one or both of the genes Slamf8 and Calhm6 as compared to a reference level.

In some embodiments, provided herein is a kit which comprises a first container and a package insert, wherein the first container comprises at least one dose of a medicament comprising an PD-1 axis binding antagonist and the package insert comprises instructions for treating a subject for cancer wherein a sample from the patient is pre-determined as having at least one of and optionally two, three, four, five or all of the following characteristics: (i) it has an increased expression level of at least one, two, or all three of the genes Irf1, Stat1, and Gbp2 as compared to a reference level; (ii) it has an increased expression level of at least 1, 2, 3, 4, 5, 6 or all 7 genes selected from the group consisting of Irf1, Stat1, Gbp2, Tap1, Psmb9, Ccl5 and Cd38 as compared to a reference level; (iii) it has an increased expression level of at least 1, 2, 3, 4, 5, 6, 7, 8, 10, 15, or all 18 genes selected from the group consisting of Irf1, Stat1, Gbp2, Tap1, Psmb9, Ccl5, Cd38, Slamf8, Calhm6, Cd40, Cxcl9, PD-L1, Nos2, M6pr, Cd74, MHCII, and Ly6l as compared to a reference level; (iv) it has an increased expression level of at least 1, 2, 3, 4, 5, 6, 7, 8, 10, 15, 20, or 25 genes selected from the group consisting of Irf1, Stat1, Gbp2, Tap1, Psmb9, Ccl5, Cd38, Slamf8, Calhm6, Cd40, Cxcl9, PD-L1, Nos2, M6pr, Cd74, MHCII, Ly6I, Cxcl10, Tor3a, Xaf1, Nlrp3, Ptgs2, Bhlhe40, Socs3, Socs1, Irf8, Cd86, Cd273, Gbp3, Plekho1, Lap3, Tnfaip2, Psme2, Atox1, Ube2I6, Scimp, Nfkbie, Tapbp, Trafd1, Rnf19b, Pomp, Dram1, and Syngr2 as compared to a reference level; (v) the sample comprises or consists of leukocytes, CD45+ cells, myeloid cells, or tumor-associated macrophages; (vi) it has an increased expression level of one or both of the genes Slamf8 and Calhm6 as compared to a reference level.

In some embodiments, in a method, medicament, PD-1 axis binding antagonist for use, or kit provided herein, the respective reference level of gene expression is determined based on an average level of the gene expression from a plurality of samples from patients having the cancer.

In some embodiments, in a method, medicament, PD-1 axis binding antagonist for use, or kit provided herein, the respective reference level of gene expression is determined based on an average level of the gene expression from a plurality of samples from human subjects.

In some embodiments, in a method, medicament, PD-1 axis binding antagonist for use, or kit provided herein, the respective reference level of gene expression is the level of gene expression of a reference gene in a cancer from the patient.

In some embodiments, in a method, medicament, PD-1 axis binding antagonist for use, or kit provided herein, the sample obtained from the patient is a tissue sample, a whole blood sample, a plasma sample, or a serum sample. Optionally, the tissue sample is a tumor tissue sample.

In some embodiments, in a method, medicament, PD-1 axis binding antagonist for use, or kit provided herein, the sample comprises or consists of tumor associated leukocytes, tumor associated CD45+ cells, tumor associated myeloid cells, or tumor associated macrophages.

In some embodiments, in a method, medicament, PD-1 axis binding antagonist for use, or kit provided herein involving leukocytes, CD45+ cells, myeloid cells, or macrophages, the cells are, respectively, tumor associated leukocytes, tumor associated CD45+ cells, tumor associated myeloid cells, or tumor associated macrophages.

In some embodiments, in a method, medicament, PD-1 axis binding antagonist for use, or kit provided herein involving an expression level of a gene, the expression level is an mRNA expression level. Optionally, the mRNA expression level is determined by RNA sequencing, RT-PCR, gene expression profiling, serial analysis of gene expression, or microarray analysis.

In some embodiments, in a method, medicament, PD-1 axis binding antagonist for use, or kit provided herein involving an expression level of a gene, the expression level is a protein expression level.

In some embodiments, in a method, medicament, PD-1 axis binding antagonist for use, or kit provided herein the PD-1 axis binding antagonist is an anti-PD-1 antibody. Optionally, the anti-PD-1 antibody is selected from the group consisting of pembrolizumab, nivolumab, cemiplimab and RN888. Optionally, the anti-PD-1 antibody comprises (a) a full length heavy chain having an amino acid sequence of SEQ ID NO: 9, and a full length light chain having an amino acid sequence of SEQ ID NO: 10; (b) a full length heavy chain having an amino acid sequence of SEQ ID NO: 7, and a full length light chain having an amino acid sequence of SEQ ID NO:8; or (c) a heavy chain variable region (VH) having an amino acid sequence of SEQ ID NO:5, and a light chain variable region (VL) of an amino acid sequence of SEQ ID NO:4.

In some embodiments, in a method, medicament, PD-1 axis binding antagonist for use, or kit provided herein, the PD-1 axis binding antagonist is an anti-PD-L1 antibody. Optionally, the anti-PD-L1 antibody is selected from the group consisting of avelumab, atezolizumab and durvalumab. Optionally, the anti-PD-L1 antibody comprises (a) a VH having an amino acid sequence of SEQ ID NO:14, and a VL having an amino acid sequences of SEQ ID NO:15; or (b) a VH having an amino acid sequence of SEQ ID NO:12, and a VL having an amino acid sequence of SEQ ID NO:13.

In some embodiments, in a method, medicament, PD-1 axis binding antagonist for use, or kit provided herein the PD-1 axis binding antagonist is administered at a dose of about 5 mg/kg, about 10 mg/kg, about 200 mg, about 240 mg, about 800 mg or about 1200 mg, and is administered about once a week, or about once every two, three, four, five weeks or six weeks.

In some embodiments, in a method, medicament, PD-1 axis binding antagonist for use, or kit provided herein, the PD-1 axis binding antagonist is avelumab or a biosimilar version thereof, which is administered intravenously at a dose selected from the group consisting of: 10 mg Q2W, 10 mg Q3W, 800 mg Q2W and 1200 mg Q3W.

In some embodiments, in a method, medicament, PD-1 axis binding antagonist for use, or kit provided herein, the PD-1 axis binding antagonist is pembrolizumab (aka MK-3475) or a biosimilar version thereof, which is administered at a dose selected from the group consisting of 1 mg/kg Q2W, 2 mg/kg Q2W, 3 mg/kg Q2W, 5 mg/kg Q2W, 10 mg Q2W, 1 mg/kg Q3W, 2 mg/kg Q3W, 3 mg/kg Q3W, 5 mg/kg Q3W, 10 mg Q3W, or a flat-dose equivalents of any of these doses, i.e., such as 200 mg Q3W. In some embodiments, MK-3475 is administered at a dose of 400 mg Q6W (400 mg flat dose every six weeks). In some embodiments, MK-3475 or a biosimilar version thereof, is administered at a dose of 200 mg Q2W for adults and 2 mg/kg (up to 200 mg) Q3W for children.

In some embodiments, in a method, medicament, PD-1 axis binding antagonist for use, or kit provided herein, the PD-1 axis binding antagonist is nivolumab or a biosimilar version thereof, which is administered at a dose of selected from the group consisting of 1 mg/kg Q2W, 2 mg/kg Q2W, 3 mg/kg Q2W, 5 mg/kg Q2W, 10 mg Q2W, 1 mg/kg Q3W, 2 mg/kg Q3W, 3 mg/kg Q3W, 5 mg/kg Q3W, and 10 mg Q3W, or a flat does equivalent of any of the forgoing doses, such as 240 mg Q2W.

In some embodiments, in a method, medicament, PD-1 axis binding antagonist for use, or kit provided herein, the PD-1 axis binding antagonist is atezolizumab or a biosimilar version thereof, which is administered at a dose of selected from the group consisting of 1 mg/kg Q2W, 2 mg/kg Q2W, 3 mg/kg Q2W, 5 mg/kg Q2W, 10 mg Q2W, 15 mg/kg Q2W 1 mg/kg Q3W, 2 mg/kg Q3W, 3 mg/kg Q3W, 5 mg/kg Q3W, and 10 mg Q3W, 15 mg/kg Q3W or a flat does equivalent of any of the forgoing doses, such as 1200 mg Q3W.

In some embodiments, in a method, medicament, PD-1 axis binding antagonist for use, or kit provided herein, the PD-1 axis binding antagonist is durvalumab or a biosimilar version thereof, which is administered at a dose of selected from the group consisting of 1 mg/kg Q2W, 2 mg/kg Q2W, 3 mg/kg Q2W, 5 mg/kg Q2W, 10 mg Q2W, 15 mg/kg Q2W 1 mg/kg Q3W, 2 mg/kg Q3W, 3 mg/kg Q3W, 5 mg/kg Q3W, and 10 mg Q3W, 15 mg/kg Q3W or a flat does equivalent of any of the forgoing doses. In some embodiments, durvalumab or a biosimilar version thereof is administered at a dose of 10 mg/kg Q2W and as an IV infusion over 60 minutes.

In some embodiments, in a method, medicament, PD-1 axis binding antagonist for use, or kit provided herein, the PD-1 axis binding antagonist is cemiplimab or a biosimilar version thereof, which is administered at a dose of selected from the group consisting of 1 mg/kg Q2W, 2 mg/kg Q2W, 3 mg/kg Q2W, 5 mg/kg Q2W, 10 mg Q2W, 15 mg/kg Q2W 1 mg/kg Q3W, 2 mg/kg Q3W, 3 mg/kg Q3W, 5 mg/kg Q3W, and 10 mg Q3W, 15 mg/kg Q3W or a flat does equivalent of any of the forgoing doses. In some embodiments, cemiplimab or a biosimilar version thereof is administered at a dose of 350 mg Q2W and as an IV infusion over 30 minutes.

In some embodiments, in a method, medicament, PD-1 axis binding antagonist for use, or kit provided herein the PD-1 axis binding antagonist is administered with at least a second anti-cancer therapeutic.

In some embodiments, in a method, medicament, PD-1 axis binding antagonist for use, or kit provided herein the cancer is advanced or metastatic solid tumor. Optionally, the cancer is bladder cancer, breast cancer, clear cell kidney cancer, lung squamous cell carcinoma, malignant melanoma, non-small-cell lung cancer (NSCLC), ovarian cancer, pancreatic cancer, prostate cancer, renal cell carcinoma, small-cell lung cancer (SCLC), triple negative breast cancer, acute lymphoblastic leukemia (ALL), acute myeloid leukemia (AML), chronic lymphocytic leukemia (CLL), chronic myeloid leukemia (CML), diffuse large B-cell lymphoma (DLBCL), follicular lymphoma, Hodgkin's lymphoma (HL), liver cancer, mantle cell lymphoma (MCL), multiple myeloma (MM), myelodysplastic syndrome (MDS), non-Hodgkin's lymphoma (NHL), Squamous Cell Carcinoma of the Head and Neck (SCCHN), small lymphocytic lymphoma (SLL), endometrial cancer, B-cell acute lymphoblastic leukemia, colorectal cancer, glioblastoma, cervical cancer, penile cancer, or non-melanoma skin cancer.

In any of the preceding embodiments involving an increased expression level of an M_3 myeloid gene (e.g. as described in the Examples herein; the genes Cxcl9, Cxcl10, Fam26f/Calhm6, Gbp3, Stat1, Plekho1, Gbp2, Lap3, PD-L1/Cd274, Cd40, Tap1, Irf1, Tnfaip2, Psme2, Atox1, Ube2I6, Scimp, Nfkbie, Psmb9, Tapbp, Trafd1, Rnf19b, Pomp, Dram1, Slamf8, Syngr2, Ccl5, Cd38, Nos2, M6pr, Tor3a, Ptgs2, Socs3, Irf8, Cd273, Cd74, MHCII, Ly6I, Xaf1, Nlrp3, Bhlhe40, Socs1, Cd86), or group of two or more M_3 myeloid associated genes in the sample obtained from a patient, expression of the gene or genes has been determined to have increased by 1% or more (e.g., 2% or more, 3% or more, 4% or more, 5% or more, 6% or more, 7% or more, 8% or more, 9% or more, 10% or more, 11% or more, 12% or more, 13% or more, 14% or more, 15% or more, 20% or more, 25% or more, 30% or more, 35% or more, 40% or more, 45% or more, or 50% or more, preferably 5% or more) relative to a reference level of the gene(s). Optionally, the reference level of an M_3 myeloid associated gene is an average gene expression level (e.g. median) across samples from multiple patients having cancer (e.g. the type of cancer to be treated in the patient). Optionally, an increased expression level is an expression level greater than the median expression level across samples from multiple patients having cancer (e.g. the type of cancer to be treated in the patient).

In any of the preceding embodiments involving an increased or high expression level of one or more M_3 myeloid associated genes in the sample obtained from a patient, expression the gene(s) has been determined to have increased by 1% or more (e.g., 2% or more, 3% or more, 4% or more, 5% or more, 6% or more, 7% or more, 8% or more, 9% or more, 10% or more, 11% or more, 12% or more, 13% or more, 14% or more, 15% or more, 20% or more, 25% or more, 30% or more, 35% or more, 40% or more, 45% or more, or 50% or more, preferably 5% or more) relative to a reference level of the respective gene(s). Optionally, the reference level of an average expression level (e.g. median) of the respective gene(s) across samples from multiple patients having cancer (e.g. the type of cancer to be treated in the patient). Optionally, an increased expression level is an expression level greater than the median expression level of the gene across samples from multiple patients having cancer (e.g. the type of cancer to be treated in the patient).

In any of the preceding embodiments, the presence and/or expression level (amount) of a gene (e.g. M_3 myeloid associated gene(s)) may be a nucleic acid expression level. In some instances, the nucleic acid expression level is determined using qPCR, rtPCR, RNA-Seq, multiplex qPCR or RT-qPCR, microarray analysis, SAGE, MassARRAY technique, or in situ hybridization (e.g., FISH).

In some embodiments, the expression of a gene is assessed in a sample that comprises or consists of tumor associated leukocytes, tumor associated CD45+ cells, tumor associated myeloid cells, or tumor associated macrophages. In some instances, the expression level of a gene is determined in tumor cells, tumor infiltrating immune cells, stromal cells, or combinations thereof.

In some embodiments, the expression level of a gene (e.g., M_3 myeloid associated gene(s)) is an mRNA expression level. Methods for the evaluation of mRNAs in cells are well known and include, for example, RNA-Seq (e.g., whole transcriptome shotgun sequencing) using next generation sequencing techniques, hybridization assays using complementary DNA probes (such as in situ hybridization using labeled riboprobes specific for the one or more genes, Northern blot and related techniques) and various nucleic acid amplification assays (such as RT-PCR using complementary primers specific for one or more of the genes, and other amplification type detection methods, such as, for example, branched DNA, SISBA, TMA and the like). In addition, such methods can include one or more steps that allow one to determine the levels of target mRNA in a biological sample (e.g., by simultaneously examining the levels a comparative control mRNA sequence of a “housekeeping” gene such as an actin family member). Optionally, the sequence of the amplified target cDNA can be determined. Optional methods include protocols that examine or detect mRNAs, such as target mRNAs, in a tissue or cell sample by microarray technologies. Using nucleic acid microarrays test and control mRNA samples from test and control tissue samples are reverse transcribed and labeled to generate cDNA probes. The probes are then hybridized to an array of nucleic acids immobilized on a solid support. The array is configured such that the sequence and position of each member of the array is known. For example, a selection of genes whose expression correlates with increased or reduced clinical benefit of treatment including a PD-1 axis binding antagonist may be arrayed on a solid support. Hybridization of a labeled probe with a particular array member indicates that the sample from which the probe was derived expresses that gene.

In some embodiments, in a method, medicament, PD-1 axis binding antagonist for use, or kit provided herein, the respective method, medicament, PD-1 axis binding antagonist for use, or kit is used to delay development of a disease or to slow the progression of a disease (e.g., a cancer, e.g., a lung cancer, a bladder cancer, a kidney cancer, or a breast cancer).

In some embodiments, in a method, medicament, PD-1 axis binding antagonist for use, or kit provided herein, application of the respective method, medicament, PD-1 axis binding antagonist for use, or kit to a patient results in an increase in progression-free survival (PFS) or overall survival (OS) in the patient. In some instances, the method, medicament, PD-1 axis binding antagonist for use, or kit may increase overall survival (OS) (e.g., by about 20% or greater, about 25% or greater, about 30% or greater, about 35% or greater, about 40% or greater, about 45% or greater, about 50% or greater, about 55% or greater, about 60% or greater, about 65% or greater, about 70% or greater, about 75% or greater, about 80% or greater, about 85% or greater, about 90% or greater, about 95% or greater, about 96% or greater, about 97% or greater, about 98% or greater, or about 99% or greater). In some instances, the method, medicament, PD-1 axis binding antagonist for use, or kit may increase OS, e.g., by about 5% to about 500%, e.g., from about 10% to about 450%, e.g., from about 20% to about 400%, e.g., from about 25% to about 350%, e.g., from about 30% to about 400%, e.g., from about 35% to about 350%, e.g., from about 40% to about 300%, e.g., from about 45% to about 250%, e.g., from about 50% to about 200%, e.g., from about 55% to about 150%, e.g., from about 60% to about 100%, e.g., from about 65% to about 100%, e.g., from about 70% to about 100%, e.g., from about 75% to about 100%, e.g., from about 80% to about 100%, e.g., from about 85% to about 100%, e.g., from about 90% to about 100%, e.g., from about 95% to about 100%, e.g., from about 98% to about 100%. In some instances, the method, medicament, PD-1 axis binding antagonist for use, or kit may increase the progression-free survival (PFS) (e.g., by about 20% or greater, about 25% or greater, about 30% or greater, about 35% or greater, about 40% or greater, about 45% or greater, about 50% or greater, about 55% or greater, about 60% or greater, about 65% or greater, about 70% or greater, about 75% or greater, about 80% or greater, about 85% or greater, about 90% or greater, about 95% or greater, about 96% or greater, about 97% or greater, about 98% or greater, or about 99% or greater). In some instances, the method, medicament, PD-1 axis binding antagonist for use, or kit may increase PFS, e.g., by about 5% to about 500%, e.g., from about 10% to about 450%, e.g., from about 20% to about 400%, e.g., from about 25% to about 350%, e.g., from about 30% to about 400%, e.g., from about 35% to about 350%, e.g., from about 40% to about 300%, e.g., from about 45% to about 250%, e.g., from about 50% to about 200%, e.g., from about 55% to about 150%, e.g., from about 60% to about 100%, e.g., from about 65% to about 100%, e.g., from about 70% to about 100%, e.g., from about 75% to about 100%, e.g., from about 80% to about 100%, e.g., from about 85% to about 100%, e.g., from about 90% to about 100%, e.g., from about 95% to about 100%, e.g., from about 98% to about 100%. In any of the above embodiments, the increase may be as compared to OS or PFS without the method, medicament, PD-1 axis binding antagonist for use, or kit, or relative to OS or PFS as compared to OS or PFS for the relevant standard of care.

In some embodiments, provided herein is a method of treating a patient having cancer, the method comprising administering to the patient a therapeutically effective amount of a PD-1 axis binding antagonist and a therapeutically effective amount of a cell-to-cell signaling antagonist. Optionally, the method further comprises administering a therapeutically effective amount of VEGFR inhibitor. Exemplary VEGFR inhibitors include sorafenib, sunitinib, tivozanib, AG13726, ABT869, and axitinib. In some embodiments, the cell-to-cell signaling antagonist is selected from the group consisting of: growth factor receptor tyroskine kinases, such as ERBB1-4 inhibitors (e.g. erlotinib, canertinib, gefitinib, lapatinib), PDGFR inhibitors (e.g. nolitinib, sorafenib, ABT869), c-Met inhibitors (e.g. PHA665752), IGF1R inhibitors (e.g. BMS536924), c-Kit inhibitors (e.g. imatinib, nolitinib), and VEGFR inhibitors.

In some embodiments, provided herein is a method of treating a patient having cancer, the method comprising administering to the patient a therapeutically effective amount of a PD-1 axis binding antagonist and a therapeutically effective amount of an epithelial-mesenchymal transition (EMT) antagonist. Optionally, the method further comprises administering a therapeutically effective amount of VEGFR inhibitor. In some embodiments, the EMT antagonist is selected from the group consisting of: galunisertib, and mTOR pathway inhibitors [e.g. rapamycin (sirolimus), RAD001 (everolimus), AP23573 (deforlimus), CI779 (temsirolimus), BEZ235, PI103].

In some embodiments, provided herein is a method of treating a patient having cancer, the method comprising administering to the patient a therapeutically effective amount of a PD-1 axis binding antagonist and a therapeutically effective amount of a cell cycle antagonist. Optionally, the method further comprises administering a therapeutically effective amount of VEGFR inhibitor. In some embodiments, the cell cycle antagonist is selected from the group consisting of: ribociclib, abemaciclib, flavopiridol, AT9283, alisertib, MK-1775, ispinesib, paclitaxel, nab-paclitaxel, docetaxel, vincristine, brentuximab, abemaciclib, PARP inhibitors (e.g. niraparib, olaparib, and rucaparib), and radiation.

In some embodiments, provided herein is a method of treating a patient having cancer, the method comprising administering to the patient a therapeutically effective amount of a PD-1 axis binding antagonist and a therapeutically effective amount morphogenesis homeobox antagonist. Optionally, the method further comprises administering a therapeutically effective amount of VEGFR inhibitor. In some embodiments, the morphogenesis homeobox antagonist is selected from the group consisting of: HXR9.

In some embodiments, provided herein is a method of treating a patient having cancer, the method comprising administering to the patient a therapeutically effective amount of a PD-1 axis binding antagonist and a therapeutically effective amount of an oxygen transport agonist. Optionally, the method further comprises administering a therapeutically effective amount of VEGFR inhibitor. In some embodiments, the oxygen transport agonist is selected from the group consisting of: hypoxia-inducible factor (HIF) inhibitors such as EZN-2968, EZN-2208, topotecan, PX-478, 2-methoxyestradiol, KC7F2, glyceollins, CAY10585, 17-AAG, 17-DMAG, Bisphenol A, BAY 87-2243, cryptotanshinone, vorinostat, LW6, HIF-1a inhibitors, PX-12, TAT-cyclo-CLLFVY, TC-S7009, PT2385, acriflavine, echinomycin, indenopyrazole 21, FM19G11, YC-1, NSC607097.

In some embodiments, provided herein is a method of treating a patient having cancer, the method comprising administering to the patient a therapeutically effective amount of a PD-1 axis binding antagonist and a therapeutically effective amount of a lipid metabolic process agonist. Optionally, the method further comprises administering a therapeutically effective amount of VEGFR inhibitor. In some embodiments, the lipid metabolic process agonist is selected from the group consisting of: thiazolidinedione (TZD), MD001, PPAR agonists.

In some embodiments, provided herein is a method of treating a patient having cancer, the method comprising administering to the patient a therapeutically effective amount of a PD-1 axis binding antagonist and a therapeutically effective amount of a notch pathway antagonist. Optionally, the cancer is urothelial carcinoma. Optionally, the notch pathway antagonist is selected from the group consisting of: OMP-21M18 (anti-DII4 mAb), A5226A (anti-nicastrin mAb), and gamma secretase inhibitors.

In some embodiments, provided herein is a method of treating a patient having cancer, the method comprising administering to the patient a therapeutically effective amount of a PD-1 axis binding antagonist and a therapeutically effective amount of a hedgehog pathway antagonist. Optionally, the cancer is urothelial carcinoma. Optionally, the hedgehog pathway antagonist is selected from the group consisting of: vismodegib and sonidegib.

In some embodiments, provided herein is a method of treating a patient having cancer, the method comprising administering to the patient a therapeutically effective amount of a PD-1 axis binding antagonist and a therapeutically effective amount of a TGFβ pathway antagonist. Optionally, the cancer is urothelial carcinoma. Optionally, the TGFβ pathway antagonist is selected from the group consisting of: 6.3G9 antibody, 264RAD, lerdelimumab, fresolimumab, ly3022859, galunisertib, and AP12009.

In some embodiments, provided herein is a method of treating a patient having cancer, the method comprising administering to the patient a therapeutically effective amount of a PD-1 axis binding antagonist and a therapeutically effective amount of an angiogenesis pathway antagonist. Optionally, the cancer is urothelial carcinoma. Optionally, the angiogenesis pathway antagonist is selected from the group consisting of: bevacizumab, brolucizumab, ranibizumab, ramucirumab, itraconazole, suramin, sorafenib, pazopanib, and everolimus.

In some embodiments, provided herein is a method for treating cancer in a patient, wherein the cancer in the patient is pre-determined to contain one or more protein altering mutations in one or more gene(s) selected from the group consisting of FRYL, FER1 L5, NBEA, SACS, CEP350, SOX17, SSPO, TENM4, KCP, ARAP1, NUP160, PTPRS, C6ORF132, KIAA1551, EPB41, MYH4, TRIM10, and TOP2B and/or to not contain a protein altering mutation in one or more gene(s) selected from the group consisting of DNAH1, AGRN, NHSL1, EPHB4, EYS, GNAS, PLXNC1, AKAP1, and TBCD; the method comprising administering to the patient a therapeutically effective amount of a PD-1 axis binding antagonist. Optionally, the cancer is urothelial carcinoma. Optionally the method further comprises administering to the patient BSC for urothelial carcinoma, as described in Example 4 herein. Optionally, the PD-1 axis binding antagonist is avelumab.

In some embodiments, provided herein is a method for treating cancer in a patient, the method comprising identifying if the cancer in the patient contains one or more protein altering mutations in one or more gene(s) selected from the group consisting of FRYL, FER1L5, NBEA, SACS, CEP350, SOX17, SSPO, TENM4, KCP, ARAP1, NUP160, PTPRS, C6ORF132, KIAA1551, EPB41, MYH4, TRIM10, and TOP2B, and if the cancer contains one or more protein altering mutations in one or more the genes, administering to the patient a therapeutically effective amount of a PD-1 axis binding antagonist. Optionally, the cancer is urothelial carcinoma. Optionally the method further comprises administering to the patient BSC for urothelial carcinoma, as described in Example 4 herein. Optionally, the PD-1 axis binding antagonist is avelumab.

In some embodiments, provided herein is a method for treating cancer in a patient, the method comprising identifying if the cancer in the patient does not contain one or more protein altering mutations in one or more gene(s) selected from the group consisting of DNAH1, AGRN, NHSL1, EPHB4, EYS, GNAS, PLXNC1, AKAP1, and TBCD, and if the cancer does not contain one or more protein altering mutations in one or more the genes, administering to the patient a therapeutically effective amount of a PD-1 axis binding antagonist. Optionally, the cancer is urothelial carcinoma. Optionally the method further comprises administering to the patient BSC for urothelial carcinoma, as described in Example 4 herein. Optionally, the PD-1 axis binding antagonist is avelumab.

### BRIEF DESCRIPTION OF THE FIGURES

FIG. 1A depicts overall survival data of patients from the IMvigor201 clinical trial treated with the PD-L1 antagonist atezolizumab, with a high, intermediate, or low combined expression of the M_3 associated genes Irf1, Stat1, and Gbp2. The X-axis depicts time (months), and the Y-axis depicts overall survival (fraction of patients; scale of 0-1; e.g. survival fraction of 0.5=50% of patients). Lines depicting patient data for high, intermediate, or low expression are labeled accordingly.

FIG. 1B depicts overall survival data of patients from the IMvigor201 clinical trial treated with the PD-L1 antagonist atezolizumab, with a high, intermediate, or low combined expression of the M_3 associated genes Irf1, Stat1, Gbp2 and Tap1. The X-axis depicts time (months), and the Y-axis depicts overall survival (fraction of patients). Lines depicting patient data for high, intermediate, or low expression are labeled accordingly.

FIG. 2 Depicts a volcano plot of association of coexpression signatures with PFS in the avelumab+axitinib combination arm of the JAVELIN Renal 101 trial. Cox Proportional Hazards model was used and <median (low) group is the reference group. Two-sided Wald test was used for p-values. Q-values were derived from multiple hypothesis adjustment using FDR.

FIG. 3 depicts the % OS over time of subjects in the JAVELIN Bladder 100 trial having at least two high-affinity FcgR variant alleles or less than two high-affinity FcgR variant alleles, treated with either avelumab+BSC or BSC alone. The lines in the graph are labeled: solid triangle: avelumab+BSC and at least two high-affinity FcgR variant alleles; empty triangle: avelumab+BSC and less than two high-affinity FcgR variant alleles; solid circle: BSC alone and at least two high-affinity FcgR variant alleles; empty circle: BSC alone and less than two high-affinity FcgR variant alleles. The Y-axis is % OS, and the X-axis is time (months).

FIG. 4A depicts OS % over time for patients in the JAVELIN Bladder 100 trial treated with avelumab+BSC or BSC alone and having JAVELIN Immuno scores less than or equal to the median. FIG. 4B depicts OS % over time for patients in the JAVELIN Bladder 100 trial treated with avelumab+BSC or BSC alone and having JAVELIN Immuno scores greater than the median. For both FIGS. 4A and 4B, the Y-axis is % OS, and the X-axis is time (months).

FIG. 5A depicts OS % over time for patients in the JAVELIN Bladder 100 trial treated with avelumab+BSC or BSC alone and having T cell-inflamed signature scores less than or equal to the median. FIG. 5B depicts OS % over time for patients in the JAVELIN Bladder 100 trial treated with avelumab+BSC or BSC alone and having T cell-inflamed signature scores greater than the median.

FIG. 6 depicts a coefficient and bootstrapping plot identifying 22 features (labeled, left side) showing the strongest association with OS benefit with avelumab/BSC vs BSC. Bootstrapping frequency (bars) associated with a biomarker indicates the probability that the biomarker would be included if the analysis were repeated multiple times. Average coefficient value is shown in each respective bar in a shaded light circle. Positive coefficients indicate that the biomarker is positively associated with the hazard for risk of death, whereas negative coefficients indicate that the biomarker is negatively associated the hazard for death.

### DETAILED DESCRIPTION

### I. Definitions

So that the invention may be more readily understood, certain technical and scientific terms are specifically defined below. Unless specifically defined elsewhere in this document, all other technical and scientific terms used herein have the meaning commonly understood by one of ordinary skill in the art to which this invention belongs.

“About” when used to modify a numerically defined parameter (e.g., the dose of a PD-1 axis binding antagonist, or the length of treatment time with a therapy described herein) means that the parameter may vary by as much as 10% below or above the stated numerical value for that parameter. For example, a dose of about 5 mg/kg may vary between 4.5 mg/kg and 5.5 mg/kg.

As used herein, including the appended claims, the singular forms of words such as “a,” “an,” and “the,” include their corresponding plural references unless the context clearly dictates otherwise.

“Administration” and “treatment,” as it applies to an animal, human, experimental subject, cell, tissue, organ, or biological fluid, refers to contact of an exogenous pharmaceutical, therapeutic, diagnostic agent, or composition to the animal, human, subject, cell, tissue, organ, or biological fluid. Treatment of a cell encompasses contact of a reagent to the cell, as well as contact of a reagent to a fluid, where the fluid is in contact with the cell. “Administration” and “treatment” also means in vitro and ex vivo treatments, e.g., of a cell, by a reagent, diagnostic, binding compound, or by another cell. The term “subject” includes any organism, preferably an animal, more preferably a mammal (e.g., rat, mouse, dog, cat, rabbit) and most preferably a human.

An “antibody” is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term encompasses not only intact polyclonal or monoclonal antibodies, but also fragments thereof (such as Fab, Fab′, F(ab′)2, Fv), single chain (scFv) and domain antibodies (including, for example, shark and camelid antibodies), and fusion proteins comprising an antibody, and any other modified configuration of the immunoglobulin molecule that comprises an antigen recognition site. An antibody includes an antibody of any class, such as IgG, IgA, or IgM (or sub-class thereof), and the antibody need not be of any particular class. Depending on the antibody amino acid sequence of the constant region of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant regions that correspond to the different classes of immunoglobulins are called alpha, delta, epsilon, gamma, and mu, respectively. The subunit structures and three-dimensional configurations of different classes of immunoglobulins are well known.

The term “antigen binding fragment” or “antigen binding portion” of an antibody, as used herein, refers to one or more fragments of an intact antibody that retain the ability to specifically bind to a given antigen (e.g., PD-1 or PD-L1). Antigen binding functions of an antibody can be performed by fragments of an intact antibody. Examples of binding fragments encompassed within the term “antigen binding fragment” of an antibody include Fab; Fab′; F(ab′)2; an Fd fragment consisting of the VH and CH1 domains; an Fv fragment consisting of the VL and VH domains of a single arm of an antibody; a single domain antibody (dAb) fragment (Ward et al., Nature 341:544-546,1989), and an isolated complementarity determining region (CDR).

An antibody, an antibody conjugate, or a polypeptide that “preferentially binds” or “specifically binds” (used interchangeably herein) to a target (e.g., PD-1 or PD-L1 protein) is a term well understood in the art, and methods to determine such specific or preferential binding are also well known in the art. A molecule is said to exhibit “specific binding” or “preferential binding” if it reacts or associates more frequently, more rapidly, with greater duration and/or with greater affinity with a particular cell or substance than it does with alternative cells or substances. An antibody “specifically binds” or “preferentially binds” to a target if it binds with greater affinity, avidity, more readily, and/or with greater duration than it binds to other substances. For example, an antibody that specifically or preferentially binds to a PD-L1 epitope is an antibody that binds this epitope with greater affinity, avidity, more readily, and/or with greater duration than it binds to other PD-L1 epitopes or non-PD-L1 epitopes. It is also understood that by reading this definition, for example, an antibody (or moiety or epitope) that specifically or preferentially binds to a first target may or may not specifically or preferentially bind to a second target. As such, “specific binding” or “preferential binding” does not necessarily require (although it can include) exclusive binding. Generally, but not necessarily, reference to binding means preferential binding.

A “variable region” of an antibody refers to the variable region of the antibody light chain or the variable region of the antibody heavy chain, either alone or in combination. As known in the art, the variable regions of the heavy and light chain each consist of four framework regions (FR) connected by three complementarity determining regions (CDRs) also known as hypervariable regions. The CDRs in each chain are held together in close proximity by the FRs and, with the CDRs from the other chain, contribute to the formation of the antigen binding site of antibodies. There are at least two techniques for determining CDRs: (1) an approach based on cross-species sequence variability (i.e., Kabat et al. Sequences of Proteins of Immunological Interest, (5th ed., 1991, National Institutes of Health, Bethesda Md.)); and (2) an approach based on crystallographic studies of antigen-antibody complexes (Al-lazikani et al., 1997, J. Molec. Biol. 273:927-948). As used herein, a CDR may refer to CDRs defined by either approach or by a combination of both approaches.

A “CDR” of a variable domain are amino acid residues within the variable region that are identified in accordance with the definitions of the Kabat, Chothia, the accumulation of both Kabat and Chothia, AbM, contact, and/or conformational definitions or any method of CDR determination well known in the art. Antibody CDRs may be identified as the hypervariable regions originally defined by Kabat et al. See, e.g., Kabat et al., 1992, Sequences of Proteins of Immunological Interest, 5th ed., Public Health Service, NIH, Washington D.C. The positions of the CDRs may also be identified as the structural loop structures originally described by Chothia and others. See, e.g., Chothia et al., Nature 342:877-883, 1989. Other approaches to CDR identification include the “AbM definition,” which is a compromise between Kabat and Chothia and is derived using Oxford Molecular's AbM antibody modeling software (now Accelrys®), or the “contact definition” of CDRs based on observed antigen contacts, set forth in MacCallum et al., J. Mol. Biol., 262:732-745, 1996. In another approach, referred to herein as the “conformational definition” of CDRs, the positions of the CDRs may be identified as the residues that make enthalpic contributions to antigen binding. See, e.g., Makabe et al., Journal of Biological Chemistry, 283:1156-1166, 2008. Still other CDR boundary definitions may not strictly follow one of the above approaches, but will nonetheless overlap with at least a portion of the Kabat CDRs, although they may be shortened or lengthened in light of prediction or experimental findings that particular residues or groups of residues or even entire CDRs do not significantly impact antigen binding. As used herein, a CDR may refer to CDRs defined by any approach known in the art, including combinations of approaches. The methods used herein may utilize CDRs defined according to any of these approaches. For any given embodiment containing more than one CDR, the CDRs may be defined in accordance with any of Kabat, Chothia, extended, AbM, contact, and/or conformational definitions.

“Isolated antibody” and “isolated antibody fragment” refers to the purification status and in such context means the named molecule is substantially free of other biological molecules such as nucleic acids, proteins, lipids, carbohydrates, or other material such as cellular debris and growth media. Generally, the term “isolated” is not intended to refer to a complete absence of such material or to an absence of water, buffers, or salts, unless they are present in amounts that substantially interfere with experimental or therapeutic use of the binding compound as described herein.

“Monoclonal antibody” or “mAb” or “Mab”, as used herein, refers to a population of substantially homogeneous antibodies, i.e., the antibody molecules comprising the population are identical in amino acid sequence except for possible naturally occurring mutations that may be present in minor amounts. In contrast, conventional (polyclonal) antibody preparations typically include a multitude of different antibodies having different amino acid sequences in their variable domains, particularly their CDRs, which are often specific for different epitopes. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al. (1975) Nature 256: 495, or may be made by recombinant DNA methods (see, e.g., U.S. Pat. No. 4,816,567). The “monoclonal antibodies” may also be isolated from phage antibody libraries using the techniques described in Clackson et al. (1991) Nature 352: 624-628 and Marks et al. (1991) J. Mol. Biol. 222: 581-597, for example. See also Presta (2005) J. Allergy Clin. Immunol. 116:731.

The term “biomarker” as used herein refers to an indicator molecule or set of molecules (e.g., predictive, diagnostic, and/or prognostic indicator), which can be detected in a sample. The biomarker may be a predictive biomarker and serve as an indicator of the likelihood of sensitivity or benefit of a patient having a particular disease or disorder (e.g., a proliferative cell disorder (e.g., cancer)) to a particular treatment (e.g. treatment with a PD-1 axis binding antagonist). Biomarkers include, but are not limited to, polynucleotides (e.g., DNA and/or RNA (e.g., mRNA)), polynucleotide copy number alterations (e.g., DNA copy numbers), polynucleotide sequence alterations (e.g. gene mutations or gene variants), polypeptides, polypeptide and polynucleotide modifications (e.g., post-translational modifications), carbohydrates, and/or glycolipid-based molecular markers. In some embodiments, a biomarker is a gene.

The terms “cancer”, “cancerous”, or “malignant” refer to or describe the physiological condition in mammals that is typically characterized by unregulated cell growth. Examples of cancer include but are not limited to, carcinoma, lymphoma, leukemia, blastoma, and sarcoma. More particular examples of such cancers include squamous cell carcinoma, myeloma, small-cell lung cancer, non-small cell lung cancer, glioma, Hodgkin's lymphoma, non-Hodgkin's lymphoma, acute myeloid leukemia (AML), multiple myeloma, gastrointestinal (tract) cancer, renal cancer, ovarian cancer, liver cancer, lymphoblastic leukemia, lymphocytic leukemia, colorectal cancer, endometrial cancer, kidney cancer, prostate cancer, thyroid cancer, melanoma, chondrosarcoma, neuroblastoma, pancreatic cancer, glioblastoma multiforme, cervical cancer, brain cancer, stomach cancer, bladder cancer, hepatoma, breast cancer, colon carcinoma, and head and neck cancer. Another particular example of cancer includes renal cell carcinoma.

“Biotherapeutic agent” means a biological molecule, such as an antibody or fusion protein, that blocks ligand/receptor signaling in any biological pathway that supports tumor maintenance and/or growth or suppresses the anti-tumor immune response.

“Chemotherapeutic agent” is a chemical compound useful in the treatment of cancer. Classes of chemotherapeutic agents include, but are not limited to: alkylating agents, antimetabolites, kinase inhibitors, spindle poison plant alkaloids, cytotoxic/antitumor antibiotics, topisomerase inhibitors, photosensitizers, anti-estrogens and selective estrogen receptor modulators (SERMs), anti-progesterones, estrogen receptor down-regulators (ERDs), estrogen receptor antagonists, leutinizing hormone-releasing hormone agonists, anti-androgens, aromatase inhibitors, EGFR inhibitors, VEGF inhibitors, and anti-sense oligonucleotides that inhibit expression of genes implicated in abnormal cell proliferation or tumor growth. Chemotherapeutic agents useful in the treatment methods of the present invention include cytostatic and/or cytotoxic agents.

“Conservatively modified variants” or “conservative substitution” refers to substitutions of amino acids in a protein with other amino acids having similar characteristics (e.g. charge, side-chain size, hydrophobicity/hydrophilicity, backbone conformation and rigidity, etc.), such that the changes can frequently be made without altering the biological activity or other desired property of the protein, such as antigen affinity and/or specificity. Those of skill in this art recognize that, in general, single amino acid substitutions in non-essential regions of a polypeptide do not substantially alter biological activity (see, e.g., Watson et al. (1987) Molecular Biology of the Gene, The Benjamin/Cummings Pub. Co., p. 224 (4th Ed.)). In addition, substitutions of structurally or functionally similar amino acids are less likely to disrupt biological activity. Exemplary conservative substitutions are set forth in Table 1 below.

TABLE 1
Exemplary Conservative Amino Acid Substitutions

| Original residue | Conservative substitution |
|---|---|
| Ala (A) | Gly; Ser |
| Arg (R) | Lys; His |
| Asn (N) | Gln; His |
| Asp (D) | Glu; Asn |
| Cys (C) | Ser; Ala |
| Gln (Q) | Asn |
| Glu (E) | Asp; Gln |
| Gly (G) | Ala |
| His (H) | Asn; Gln |
| Ile (I) | Leu; Val |
| Leu (L) | Ile; Val |
| Lys (K) | Arg; His |
| Met (M) | Leu; Ile; Tyr |
| Phe (F) | Tyr; Met; Leu |
| Pro (P) | Ala |
| Ser (S) | Thr |
| Thr (T) | Ser |
| Trp (W) | Tyr; Phe |
| Tyr (Y) | Trp; Phe |
| Val (V) | Ile; Leu |

“Genetic mutation”, or “genetic alteration”, as used here in, refer to a germline, somatic or recombinant mutation of a wild type gene, including substitution, insertion, and deletion of one or more nucleotides in the gene's coding or non-coding sequence.

“A protein altering mutation” as used herein refers to a genetic mutation that (a) results in a change in the amino acid sequence of the corresponding protein; or (b) otherwise results in a disruption of the expression, or function of the protein which the gene encodes. Examples of a protein altering genetic mutation includes but is not limited to disruptive inframe deletion, disruptive inframe insertion, frameshift variant, inframe deletion, inframe insertion, initiator codon variant, intron variant, missense variant, non-canonical start codon, splice acceptor variant, splice donor variant, splice region variant, start lost, stop gained, stop lost, and stop retained variant. Any reference to a “mutation” in a gene herein may include a protein altering mutation, unless the context clearly dictates otherwise.

“Expression level”, “level of expression” and the like refers to the amount of a biomarker in a biological sample. “Expression” generally refers to the process by which information (e.g., gene-encoded and/or epigenetic information) is converted into the structures present and operating in the cell. Therefore, as used herein, “expression” may refer to transcription into a polynucleotide, translation into a polypeptide, or even polynucleotide and/or polypeptide modifications (e.g., posttranslational modification of a polypeptide). Fragments of the transcribed polynucleotide, the translated polypeptide, or polynucleotide and/or polypeptide modifications (e.g., posttranslational modification of a polypeptide) shall also be regarded as expressed whether they originate from a transcript generated by alternative splicing or a degraded transcript, or from a posttranslational processing of the polypeptide, e.g., by proteolysis. “Expressed genes” include those that are transcribed into a polynucleotide as mRNA and then translated into a polypeptide, and also those that are transcribed into RNA but not translated into a polypeptide (for example, transfer and ribosomal RNAs).

“Increased expression”, “increased expression level”, “increased levels”, “elevated expression”, “elevated expression levels”, or “elevated levels” refers to an increased expression or increased levels of a biomarker in an individual relative to a reference level or control, such as an individual or individuals who do not have the disease or disorder (e.g., cancer), an internal control (e.g., a housekeeping biomarker), or a median expression level of the biomarker in samples from a group/population of patients.

“Decreased expression”, “decreased expression level”, “decreased levels”, “reduced expression”, “reduced expression levels”, or “reduced levels” refers to a decrease expression or decreased levels of a biomarker in an individual relative to a reference level or control, such as an individual or individuals who do not have the disease or disorder (e.g., cancer), an internal control (e.g., a housekeeping biomarker), or a median expression level of the biomarker in samples from a group/population of patients. In some embodiments, reduced expression is little or no expression.

“Housekeeping gene” refers herein to a gene or group of genes that encode proteins whose activities are essential for the maintenance of cell function and which are typically similarly present in all cell types. In some embodiments, the housekeeping gene can be beta actin (ACTB), glyceraldehyde 3-phosphate dehydrogenase (GAPDH), phosphoglycerate kinase 1 (PGK1), heterogenous nuclear ribonucleoprotein L (HNRNPL), poly-binding protein 1 (PCBP1), or retention in endoplasmic reticulum sorting receptor 1 (RER1).

“Patient” or “subject” refers to any single subject for which therapy is desired or that is participating in a clinical trial, epidemiological study or used as a control, including humans and mammalian veterinary patients such as cattle, horses, dogs, and cats.

“PD-1 axis binding antagonist” refers to a molecule that inhibits the interaction of a PD-1 axis binding partner with either one or more of its binding partner, so as to remove T-cell dysfunction resulting from signaling on the PD-1 signaling axis, with a result being to restore or enhance T-cell function. As used herein, a PD-1 axis binding antagonist includes a PD-1 antagonist, a PD-L1 antagonist and a PD-L2 antagonist.

“PD-L1 antagonist” means any chemical compound or biological molecule that blocks binding of PD-L1 expressed on a cancer cell to PD-1. In any of the treatment method, medicaments and uses of the present invention in which a human subject is being treated, the PD-L1 antagonist blocks binding of human PD-L1 to human PD-1.

PD-L1 antagonists useful in the any of the treatment methods, medicaments, and uses of the present invention include a monoclonal antibody (mAb) which specifically binds to PD-L1, and preferably specifically binds to human PD-L1. The mAb may be a human antibody, a humanized antibody or a chimeric antibody, and may include a human constant region. In some embodiments the human constant region is selected from the group consisting of IgG1, IgG2, IgG3 and IgG4 constant regions, and in preferred embodiments, the human constant region is an IgG1 or IgG4 constant region. In some embodiments, the antigen binding fragment is selected from the group consisting of Fab, Fab′-SH, F(ab′)2, scFv and Fv fragments. As used herein, an anti-human PD-L1 antibody refers to an antibody that specifically binds to mature human PD-L1. A mature human PD-L1 molecule consists of amino acids 19-290 of the following sequence (SEQ ID NO: 16):

(SEQ ID NO: 16)
MRIFAVFIFMTYWHLLNAFTVTVPKDLYVVEYGSNMTIECKFPVEKQLD
LAALIVYWEMEDKNIIQFVHGEEDLKVQHSSYRQRARLLKDQLSLGNAA
LQITDVKLQDAGVYRCMISYGGADYKRITVKVNAPYNKINQRILVVDPV
TSEHELTCQAEGYPKAEVIWTSSDHQVLSGKTTTTNSKREEKLFNVTST
LRINTTTNEIFYCTFRRLDPEENHTAELVIPELPLAHPPNERTHLVILG
AILLCLGVALTFIFRLRKGRMMDVKKCGIQDTNSKKQSDTHLEET.

Examples of PD-1 axis binding antagonist and useful in the treatment method, medicaments and uses of the present invention, are described in WO2013079174, WO2015061668, WO2010089411, WO/2007/005874, WO/2010/036959, WO/2014/100079, WO2013/019906, WO/2010/077634, and U.S. Pat. Nos. 8,552,154, 8,779,108, and 8,383,796. Specific PD-1 axis binding antagonist useful in the treatment method, medicaments and uses of the present invention include, for example without limitation: pembrolizumab (aka MK-3475, an anti-PD-1 IgG4 monoclonal antibody) nivolumab (aka BMS-936558 or MDX1106, an anti-PD-1 IgG4 monoclonal antibody), cemiplimab (aka REGN-2810, an anti-PD-1 antibody), avelumab (aka MSB0010718C, an anti-PD-L1 IgG1 monoclonal antibody), atezolizumab (aka MPDL3280A an IgG1-engineered, anti-PD-L1 antibody), BMS-936559 (a fully human, anti-PD-L1, IgG4 monoclonal antibody), MED14736 (aka durvalumab, an engineered IgG1 kappa anti-PD-L1 monoclonal antibody with triple mutations in the Fc domain to remove antibody-dependent, cell-mediated cytotoxic activity). Additional exemplary PD-1 axis binding antagonist useful in the treatment method, medicaments and uses of the present invention include SHR1210 (anti-PD-1 antibody), KN035 (anti-PD-L1 antibody), 161308 (anti-PD-1 antibody), PDR001 (anti-PD-1 antibody), BGB-A317 (anti-PD-1 antibody), BCD-100 (anti-PD-1 antibody), JS001 (anti-PD-1 antibody), as described in Darvin et al. Experimental & Molecular Medicine (2018) 50:165, the disclosure of which is herein incorporated by reference in its entirety. In some embodiments, a PD-1 axis binding antagonist is a small molecule PD-1 or PD-L1 antagonist (e.g. CA-170), as described in Yang et al Med. Res. Rev. (2019), 39, pp 265-301, the disclosure of which is herein incorporated by reference in its entirety.

Other PD-1 axis binding antagonist useful in the any of the treatment method, medicaments and uses of the present invention also include an immunoadhesin that specifically binds to PD-1 or PD-L1, and preferably specifically binds to human PD-1 or PD-L1, e.g., a fusion protein containing a portion that binds to PD-1 or PD-L1, fused to a constant region such as an Fc region of an immunoglobulin molecule.

Table 2 below provides sequences of some of the exemplary antibodies that are PD-1 axis binding antagonist for use in the treatment method, medicaments and uses of the present invention. CDRs are underlined for mAb7 and mAb15. The mAB7 is also known as RN888 or PF-6801591. mAb7 (aka RN888) and mAb15 are disclosed in International Patent Publication No. WO2016/092419, the disclosure of which is hereby incorporated by reference in its entirety.

TABLE 2
Sequences of Exemplary PD-1 Axis Binding Antagonist

| | |
|---|---|
| mAb7(aka RN888) | QVQLVQSGAEVKKPGASVKVSCKASGYTFTSYWINWVRQAPG |
| or mAb15 full- | QGLEWMGNIYPGSSLTNYNEKFKNRVTMTRDTSTSTVYMELSS |
| length heavy chain | LRSEDTAVYYCARLSTGTFAYWGQGTLVTVSSASTKGPSVFPL |
| | APCSRSTSESTAALGCLVKDYFPEPVTVSWNSGALTSGVHTFP |
| | AVLQSSGLYSLSSVVTVPSSSLGTKTYTCNVDHKPSNTKVDKR |
| | VESKYGPPCPPCPAPEFLGGPSVFLFPPKPKDTLMISRTPEVTC |
| | VVVDVSQEDPEVQFNWYVDGVEVHNAKTKPREEQFNSTYRVV |
| | SVLTVLHQDWLNGKEYKCKVSNKGLPSSIEKTISKAKGQPREP |
| | QVYTLPPSQEEMTKNQVSLTCLVKGFYPSDIAVEWESNGQPEN |
| | NYKTTPPVLDSDGSFFLYSRLTVDKSRWQEGNVFSCSVMHEAL |
| | HNHYTQKSLSLSLGK (SEQ ID NO: 1) |
| mAb7 or mAb 15 | QVQLVQSGAEVKKPGASVKVSCKASGYTFTSYWINWVRQAPG |
| full-length heavy | QGLEWMGNIYPGSSLTNYNEKFKNRVTMTRDTSTSTVYMELSS |
| chain without the C- | LRSEDTAVYYCARLSTGTFAYWGQGTLVTVSSASTKGPSVFPL |
| terminal lysine | APCSRSTSESTAALGCLVKDYFPEPVTVSWNSGALTSGVHTFP |
| | AVLQSSGLYSLSSVVTVPSSSLGTKTYTCNVDHKPSNTKVDKR |
| | VESKYGPPCPPCPAPEFLGGPSVFLFPPKPKDTLMISRTPEVTC |
| | VVVDVSQEDPEVQFNWYVDGVEVHNAKTKPREEQFNSTYRVV |
| | SVLTVLHQDWLNGKEYKCKVSNKGLPSSIEKTISKAKGQPREP |
| | QVYTLPPSQEEMTKNQVSLTCLVKGFYPSDIAVEWESNGQPEN |
| | NYKTTPPVLDSDGSFFLYSRLTVDKSRWQEGNVFSCSVMHEAL |
| | HNHYTQKSLSLSLG (SEQ ID NO: 2) |
| mAb7 full-length | DIVMTQSPDSLAVSLGERATINCKSSQSLWDSGNQKNFLTWYQ |
| light chain | QKPGQPPKLLIYWTSYRESGVPDRFSGSGSGTDFTLTISSLQAE |
| | DVAVYYCQNDYFYPHTFGGGTKVEIKRGTVAAPSVFIFPPSDE |
| | QLKSGTASVVCLLNNFYPREAKVQWKVDNALQSGNSQESVTE |
| | QDSKDSTYSLSSTLTLSKADYEKHKVYACEVTHQGLSSPVTKS |
| | FNRGEC (SEQ ID NO: 3) |
| mAb7 light chain | DIVMTQSPDSLAVSLGERATINCKSSQSLWDSGNQKNFLTWYQ |
| variable region | QKPGQPPKLLIYWTSYRESGVPDRFSGSGSGTDFTLTISSLQAE |
| | DVAVYYCQNDYFYPHTFGGGTKVEIK (SEQ ID NO: 4) |
| mAB7 and mAB15 | QVQLVQSGAEVKKPGASVKVSCKASGYTFTSYWINWVRQAPG |
| heavy chain | QGLEWMGNIWPGSSLTNYNEKFKNRVTMTRDTSTSTVYMELS |
| variable region | SLRSEDTAVYYCARLLTGTFAYWGQGTLVTVSS (SEQ ID NO: |
| | 5) |
| mAb15 light chain | DIVMTQSPDSLAVSLGERATINCKSSQSLWDSGNQKNFLTWYQ |
| variable region | QKPGQPPKLLIYWTSYRESGVPDRFSGSGSGTDFTLTISSLQAE |
| | DVAVYYCQNDYFYPHTFGGGTKVEIK (SEQ ID NO: 6) |
| Nivolumab, | QVQLVESGGGWQPGRSLRLDCKASGITFSNSGMHWVRQAPG |
| MDX1106, full | KGLEWVAVrWYDGSKRYYADSVKGRFTISRDNSKNTLFLQMNS |
| length heavy chain | LRAEDTAVYYCATNDDYWGQGTLVTVSSASTKGPSVFPLAPCS |
| From | RSTSESTAALGCLVDYFPEPVTVSWNSGALTSGVHTFPAVLQS |
| WO2006/121168 | SGLYSLSSVVTVPSSSLGTTYTCNVDHKPSNTKVDRVESYGPP |
| | CPPCPAPEFLGGPSVFLFPPKPKDTLMISRTPEVTCWVDVSQE |
| | DPEVQFNWYYDGVEVHNATKPREEQFNSTYRVVSVLTVLHQD |
| | WLNGKEYKCKVSNKGLPSSIEKTISKA |
| | GQPREPQVYTLPPSQEEMTKNQVSLTCLVKGFYPSDIAVEWES |
| | NGQPEKNYKTTPPVLDSDGSFFLYSRLTVDKSRWQEGNVFSC |
| | SVMHEALHNHYTQKSLSLSLGK (SEQ ID NO: 7) |
| Nivolumab, | EIVLTQSPATLSLSPGERATLSCRASQSVSSYLAWYQQPGQAP |
| MDX1106, full | RLLIYDASNRATGIPARFSGSGSGTDFTLTISSLEPEDFAVYYCQ |
| length light chain | QSSNWPRTFGQGTKVEIRTVAAPSVFIFPPSDEQLSGTASVVCL |
| From | LNNFYPREAVQWKVDNALQSGNSQESVTEQDSDSTYSLSSTL |
| WO2006/121168 | TLSKADYEKHKVYACEVTHQGLSSPVT SFNRGEC (SEQ ID |
| | NO: 8) |
| Pembrolizumab, | QVQLVQSGVEVKKPGASVKVSCKASGYTFTNYYMYWVRQA |
| MK3475, full length | PGQGLEWMGGINPSNGGTNFNEKFKNRVTLTTDSSTTTAYM |
| heavy chain | ELKSLQFDDTAVYYCARRDYRFDMGFDYWGQGTTVTVSSA |
| From | STKGPSVFPLAPCSRSTSESTAALGCLVKDYFPEPVTVSWNS |
| WO2009114335 | GALTSGVHTFPAVLQSSGLYSLSSVVTVPSSSLGTKTYTCNV |
| | DHKPSNTKVDKRVESKYGPPCPPCPAPEFLGGPSVFLFPPK |
| | PKDTLMISRTPEVTCVVVDVSQEDPEVQFNWYVDGVEVHNA |
| | KTKPREEQFNSTYRVVSVLTVLHQDWLNGKEYKCKVSNKGL |
| | PSSIEKTISKAKGQPREPQVYTLPPSQEEMTKNQVSLTCLVK |
| | GFYPSDIAVEWESNGQPENNYKTTPPVLDSDGSFFLYSRLTV |
| | DKSRWQEGNVFSCSVMHEALHNHYTQKSLSLSLGK (SEQ |
| | ID NO: 9) |
| Pembrolizumab, | EIVLTQSPATLSLSPGERATLSCRASKGVSTSGYSYLHWYQQ |
| MK3475, full length | KPGQAPRLLIYLASYLESGVPARFSGSGSGTDFTLTISSLEPE |
| light chain | DFAVYYCQHSRDLPLTFGGGTKVEIKRTVAAPSVFIFPPSDE |
| From | QLKSGTASVVCLLNNFYPREAKVQWKVDNALQSGNSQESVT |
| WO2009114335 | EQDSKDSTYSLSSTLTLSKADYEKHKVYACEVTHQGLSSPVT |
| | KSFNRGEC (SEQ ID NO: 10) |
| AMP224, without | LFTVTVPKELYIIEHGSNVTLECNFDTGSHVNLGAITASLQKVEN |
| signal sequence | DTSPHRERATLLEEQLPLGKASFHIPQVQVRDEGQYQCIIIYGVA |
| From | WDYKYLTLKVKASYRKINTHILKVPETDEVELTCQATGYPLAEV |
| WO2010027827 | SWPNVSVPANTSHSRTPEGLYQVTSVLRLKPPPGRNFSCVFW |
| and | NTHVRELTLASIDLQSQMEPRTHPTWEPKSCDKTHTCPPCPAP |
| WO2011066342 | ELLGGPSVFLFPPKPKDTLMISRTPEVTCWVDVSHEDPEVKFN |
| | WYVDGVEVHNAKTKPREEQYNSTYRWSVLTVLHQDWLNGKE |
| | YKCKVSNKALPAPIEKTISKAKGQPREPQVYTLPPSRDELTKNQ |
| | VSLTCLVKGFYPSDIAVEWESNGQPENNYKTTPPVLDSDGS |
| | FFLYSKLTVDKSRWQQGNVFSCSVMHEALHNHYTQKSLSLSP |
| | GK (SEQ ID NO: 11) |
| YW243.55.S70 | EVQLVESGGGLVQPGGSLRLSCAASGFTFSDSWIHWVRQAPG |
| heavy chain | KGLEWVAWISPYGGSTYYADSVKGRFTISADTSKNTAYLQMNS |
| From | LRAEDTAVYYCARRHWPGGFDYWGQGTLVTVSA (SEQ ID NO: |
| WO2010077634 | 12) |
| YW243.55.S70 light | DIQMTQSPSSLSASVGDRVTITCRASQDVSTAVAWYQQKPGKA |
| chain | PKLLIYSASFLYSGVPSRFSGSGSGTDFTLTISSLQPEDFATYYC |
| From | QQYLYH PATFGQGTKVEIKR (SEQ ID NO: 13) |
| WO2010077634 | |
| avelumab heavy | EVQLLESGGGLVQPGGSLRLSCAASGFTFSSYIMWVRQAPGK |
| chain variable | GLEWVSSIYPSGGITFYADKGRFTISRDNSKNTLYLQMNSLRAE |
| region | DTAVYYCARIKLGTVTTVDYWGQ GTLVTVSS (SEQ ID NO: 14) |
| From WO13079174 | |
| avelumab light | QSALTQPASVSGSPGQSITISCTGTSSDVGGYNYVSWYQQHP |
| chain variable | GKAPKLMIYDVSNRPSGVSNRFSGSKSGNTASLTISGLQAEDE |
| region | ADYYCSSYTSSSTRVFGTGTKVTVL (SEQ ID NO: 15) |
| From WO13079174 | |

Table 3 below provides the sequences of the anti-PD-L1 antibody avelumab for use in the treatment methods, medicaments and uses of the present invention. Avelumab is disclosed as A09-246-2, in International Patent Publication No. WO2013/079174, the disclosure of which is hereby incorporated by reference in its entirety.

TABLE 3
ANTI-HUMAN PD-L1 MONOCLONAL ANTIBODY AVELUMAB
SEQUENCES

| | |
|---|---|
| Heavy chain | SYIMM (SEQ ID NO: 17) |
| CDR1 (CDRH1) | |
| Heavy chain | SIYPSGGITFY (SEQ ID NO: 18) |
| CDR2 (CDRH2) | |
| Heavy chain | IKLGTVTTVDY (SEQ ID NO: 19) |
| CDR3 (CDRH3) | |
| Light chain | TGTSSDVGGYNYVS (SEQ ID NO: 20) |
| CDR1 (CDRL1) | |
| Light chain | DVSNRPS (SEQ ID NO: 21) |
| CDR2 (CDRL2) | |
| Light chain | SSYTSSSTRV (SEQ ID NO: 22) |
| CDR3 (CDRL3) | |
| Heavy chain | EVQLLESGGGLVQPGGSLRLSCAASGFTFSSYIMMWVR |
| variable region | QAPGKGLEWVSSIYPSGGITFYADKGRFTISRDNSKNTL |
| (VR) | YLQMNSLRAEDTAVYYCARIKLGTVTTVDYWGQGTLVT |
| | VSS (SEQ ID NO: 14) |
| Light chain VR | QSALTQPASVSGSPGQSITISCTGTSSDVGGYNYVSWY |
| | QQHPGKAPKLMIYDVSNRPSGVSNRFSGSKSGNTASLTI |
| | SGLQAEDEADYYCSSYTSSSTRVFGTGTKVTVL (SEQ |
| | ID NO: 15) |
| Heavy chain | EVQLLESGGGLVQPGGSLRLSCAASGFTFSSYIMMWVR |
| | QAPGKGLEWVSSIYPSGGITFYADTVKGRFTISRDNSKN |
| | TLYLQMNSLRAEDTAVYYCARIKLGTVTTVDYWGQGTLV |
| | TVSSASTKGPSVFPLAPSSKSTSGGTAALGCLVKDYFPE |
| | PVTVSWNSGALTSGVHTFPAVLQSSGLYSLSSVVTVPS |
| | SSLGTQTYICNVNHKPSNTKVDKKVEPKSCDKTHTCPPC |
| | PAPELLGGPSVFLFPPKPKDTLMISRTPEVTCVVVDVSH |
| | EDPEVKFNWYVDGVEVHNAKTKPREEQYNSTYRVVSVL |
| | TVLHQDWLNGKEYKCKVSNKALPAPIEKTISKAKGQPRE |
| | PQVYTLPPSRDELTKNQVSLTCLVKGFYPSDIAVEWESN |
| | GQPENNYKTTPPVLDSDGSFFLYSKLTVDKSRWQQGNV |
| | FSCSVMHEALHNHYTQKSLSLSPGK (SEQ ID NO: 23) |
| Light chain | QSALTQPASVSGSPGQSITISCTGTSSDVGGYNYVSWY |
| | QQHPGKAPKLMIYDVSNRPSGVSNRFSGSKSGNTASLTI |
| | SGLQAEDEADYYCSSYTSSSTRVFGTGTKVTVLGQPKA |
| | NPTVTLFPPSSEELQANKATLVCLISDFYPGAVTVAWKA |
| | DGSPVKAGVETTKPSKQSNNKYAASSYLSLTPEQWKSH |
| | RSYSCQVTHEGSTVEKTVAPTECS (SEQ ID NO: 24) |

“PD-L1” expression as used herein means any detectable level of expression of PD-L1 protein on the cell surface or of PD-L1 mRNA within a cell or tissue. PD-L1 protein expression may be detected with a diagnostic PD-L1 antibody in an IHC assay of a tumor tissue section or by flow cytometry. Alternatively, PD-L1 protein expression by tumor cells may be detected by PET imaging, using a binding agent (e.g., antibody fragment, affibody and the like) that specifically binds to PD-L1. Techniques for detecting and measuring PD-L1 mRNA expression include RT-PCR and real-time quantitative RT-PCR.

Several approaches have been described for quantifying PD-L1 protein expression in IHC assays of tumor tissue sections. See, e.g., Thompson, R. H., et al., PNAS 101 (49); 17174-17179 (2004); Thompson, R. H. et al., Cancer Res. 66:3381-3385 (2006); Gadiot, J., et al., Cancer 117:2192-2201 (2011); Taube, J. M. et al., Sci Transl Med 4, 127ra37 (2012); and Toplian, S. L. et al., New Eng. J Med. 366 (26): 2443-2454 (2012).

One approach employs a simple binary end-point of positive or negative for PD-L1 expression, with a positive result defined in terms of the percentage of tumor cells that exhibit histologic evidence of cell-surface membrane staining. A tumor tissue section is counted as positive for PD-L1 expression is at least 1%, and preferably 5% of total tumor cells.

In another approach, PD-L1 expression in the tumor tissue section is quantified in the tumor cells as well as in infiltrating immune cells, which predominantly comprise lymphocytes. The percentage of tumor cells and infiltrating immune cells that exhibit membrane staining are separately quantified as <5%, 5 to 9%, and then in 10% increments up to 100%. For tumor cells, PD-L1 expression is counted as negative if the score is <5% score and positive if the score is ≥5%. PD-L1 expression in the immune infiltrate is reported as a semi-quantitative measurement called the adjusted inflammation score (AIS), which is determined by multiplying the percent of membrane staining cells by the intensity of the infiltrate, which is graded as none (0), mild (score of 1, rare lymphocytes), moderate (score of 2, focal infiltration of tumor by lymphohistiocytic aggregates), or severe (score of 3, diffuse infiltration). A tumor tissue section is counted as positive for PD-L1 expression by immune infiltrates if the AIS is 5.

The level of PD-L1 mRNA expression may be compared to the mRNA expression levels of one or more reference genes that are frequently used in quantitative RT-PCR, such as ubiquitin C.

In some embodiments, a level of PD-L1 expression (protein and/or mRNA) by malignant cells and/or by infiltrating immune cells within a tumor is determined to be “overexpressed” or “elevated” based on comparison with the level of PD-L1 expression (protein and/or mRNA) by an appropriate control. For example, a control PD-L1 protein or mRNA expression level may be the level quantified in nonmalignant cells of the same type or in a section from a matched normal tissue.

“RECIST 1.1 Response Criteria” as used herein means the definitions set forth in Eisenhauer et al., E. A. et al., Eur. J Cancer 45:228-247 (2009) for target lesions or nontarget lesions, as appropriate based on the context in which response is being measured.

“Sustained response” means a sustained therapeutic effect after cessation of treatment with a therapeutic agent, or a therapy described herein. In some embodiments, the sustained response has a duration that is at least the same as the treatment duration, or at least 1.5, 2.0, 2.5 or 3 times longer than the treatment duration.

An “effective response” of a patient or a patient's “responsiveness” to treatment with a medicament and similar wording refers to the clinical or therapeutic benefit imparted to a patient at risk for, or having a, a disease or disorder, such as cancer. In one embodiment, such benefit includes any one or more of: extending survival (including overall survival and/or progression-free survival); resulting in an objective response (including a complete response or a partial response); or improving signs or symptoms of cancer.

“Tissue Section” refers to a single part or piece of a tissue sample, e.g., a thin slice of tissue cut from a sample of a normal tissue or of a tumor.

“Treat” or “treating” a cancer as used herein means to administer a therapy of a PD-1 axis binding antagonist to a subject having a cancer, or diagnosed with a cancer, to achieve at least one positive therapeutic effect, such as for example, reduced number of cancer cells, reduced tumor size, reduced rate of cancer cell infiltration into peripheral organs, or reduced rate of tumor metastasis or tumor growth. Positive therapeutic effects in cancer can be measured in a number of ways (See, W. A. Weber, J. Nucl. Med. 50:1S-10S (2009)). For example, with respect to tumor growth inhibition, according to National Cancer Institute (NCI) standards, a T/C less than or equal to 42% is the minimum level of anti-tumor activity. A T/C<10% is considered a high anti-tumor activity level, with T/C (%)=Median tumor volume of the treated/Median tumor volume of the control×100. In some embodiments, the treatment achieved by a combination of the invention is any of partial response (PR), complete response (CR), overall response (OR), progression free survival (PFS), disease free survival (DFS) and overall survival (OS). PFS, also referred to as “Time to Tumor Progression” indicates the length of time during and after treatment that the cancer does not grow, and includes the amount of time patients have experienced a CR or PR, as well as the amount of time patients have experienced stable disease (SD). DFS refers to the length of time during and after treatment that the patient remains free of disease. OS refers to a prolongation in life expectancy as compared to naive or untreated subjects or patients. In some embodiments, response to a combination of the invention is any of PR, CR, PFS, DFS, OR, or OS that is assessed using Response Evaluation Criteria in Solid Tumors (RECIST) 1.1 response criteria. The treatment regimen for a combination of the invention that is effective to treat a cancer patient may vary according to factors such as the disease state, age, and weight of the patient, and the ability of the therapy to elicit an anti-cancer response in the subject. While an embodiment of any of the aspects of the invention may not be effective in achieving a positive therapeutic effect in every subject, it should do so in a statistically significant number of subjects as determined by any statistical test known in the art such as the Student's t-test, the chi2-test, the U-test according to Mann and Whitney, the Kruskal-Wallis test (H-test), Jonckheere-Terpstra-test and the Wilcoxon-test.

The terms “treatment regimen”, “dosing protocol” and dosing regimen are used interchangeably to refer to the dose and timing of administration of each therapeutic agent in a combination of the invention.

As used herein, “treatment” is an approach for obtaining beneficial or desired clinical results. For purposes of this invention, beneficial or desired clinical results include, but are not limited to, one or more of the following: reducing the proliferation of (or destroying) neoplastic or cancerous cells, inhibiting metastasis of neoplastic cells, shrinking or decreasing the size of tumor, remission of a PD-1 axis associated disease (e.g., cancer), decreasing symptoms resulting from a PD-1 axis associated disease (e.g., cancer), increasing the quality of life of those suffering from a PD-1 axis associated disease (e.g., cancer), decreasing the dose of other medications required to treat a PD-1 axis associated disease (e.g., cancer), delaying the progression of a PD-1 axis associated disease (e.g., cancer), curing a PD-1 axis associated disease (e.g., cancer), and/or prolong survival of patients having a PD-1 axis associated disease (e.g., cancer).

“Ameliorating” means a lessening or improvement of one or more symptoms as compared to not administering a therapy or medicament. “Ameliorating” also includes shortening or reduction in duration of a symptom.

As used herein, an “effective dosage” or “effective amount” of drug, compound, or pharmaceutical composition is an amount sufficient to effect any one or more beneficial or desired results. For prophylactic use, beneficial or desired results include eliminating or reducing the risk, lessening the severity, or delaying the outset of the disease, including biochemical, histological and/or behavioral symptoms of the disease, its complications and intermediate pathological phenotypes presenting during development of the disease. For therapeutic use, beneficial or desired results include clinical results such as reducing incidence or amelioration of one or more symptoms of various PD-1 axis associated diseases or conditions (such as for example advanced RCC), decreasing the dose of other medications required to treat the disease, enhancing the effect of another medication, and/or delaying the progression of the PD-1 axis associated disease of patients. An effective dosage can be administered in one or more administrations. For purposes of this invention, an effective dosage of drug, compound, or pharmaceutical composition is an amount sufficient to accomplish prophylactic or therapeutic treatment either directly or indirectly. As is understood in the clinical context, an effective dosage of a drug, compound, or pharmaceutical composition may or may not be achieved in conjunction with another drug, compound, or pharmaceutical composition. Thus, an “effective dosage” may be considered in the context of administering one or more therapeutic agents, and a single agent may be considered to be given in an effective amount if, in conjunction with one or more other agents, a desirable result may be or is achieved. In reference to the treatment of cancer, an effective amount refers to that amount which has the effect of (1) reducing the size of the tumor, (2) inhibiting (that is, slowing to some extent, preferably stopping) tumor metastasis emergence, (3) inhibiting to some extent (that is, slowing to some extent, preferably stopping) tumor growth or tumor invasiveness, and/or (4) relieving to some extent (or, preferably, eliminating) one or more signs or symptoms associated with the cancer. Therapeutic or pharmacological effectiveness of the doses and administration regimens may also be characterized as the ability to induce, enhance, maintain or prolong disease control and/or overall survival in patients with these specific tumors, which may be measured as prolongation of the time before disease progression

The terms “for improving progression free survival” in the context of the present invention refer, with respect to a patient within a patient group, to the average length of time during and after treatment in which a patient's disease does not get worse. As the skilled person will appreciate, a patient's progression free survival is improved or enhanced, if the patient belongs to a subgroup of patients that has a significantly longer mean length of time during which the disease does not get worse compared to another subgroup of patients.

“Tumor” as it applies to a subject diagnosed with, or suspected of having, a cancer refers to a malignant or potentially malignant neoplasm or tissue mass of any size, and includes primary tumors and secondary neoplasms. A solid tumor is an abnormal growth or mass of tissue that usually does not contain cysts or liquid areas. Different types of solid tumors are named for the type of cells that form them. Examples of solid tumors are sarcomas, carcinomas, and lymphomas. Leukemias (cancers of the blood) generally do not form solid tumors (National Cancer Institute, Dictionary of Cancer Terms).

“Tumor burden” also referred to as “tumor load”, refers to the total amount of tumor material distributed throughout the body. Tumor burden refers to the total number of cancer cells or the total size of tumors, throughout the body, including lymph nodes and bone narrow. Tumor burden can be determined by a variety of methods known in the art, such as, e.g. by measuring the dimensions of tumors upon removal from the subject, e.g., using calipers, or while in the body using imaging techniques, e.g., ultrasound, bone scan, computed tomography (CT) or magnetic resonance imaging (MRI) scans.

The term “tumor size” refers to the total size of the tumor which can be measured as the length and width of a tumor. Tumor size may be determined by a variety of methods known in the art, such as, e.g. by measuring the dimensions of tumors upon removal from the subject, e.g., using calipers, or while in the body using imaging techniques, e.g., bone scan, ultrasound, CT or MRI scans.

“Variable regions” or “V region” as used herein means the segment of IgG chains which is variable in sequence between different antibodies. It extends to Kabat residue 109 in the light chain and 113 in the heavy chain.

“M_3 myeloid gene(s)” refers to any of the genes: Cxcl9, Cxcl10, Fam26f/Calhm6, Gbp3, Stat1, Plekho1, Gbp2, Lap3, PD-L1/Cd274, Cd40, Tap1, Irf1, Tnfaip2, Psme2, Atox1, Ube2I6, Scimp, Nfkbie, Psmb9, Tapbp, Trafd1, Rnf19b, Pomp, Dram1, Slamf8, Syngr2, Ccl5, Cd38, Nos2, M6pr, Tor3a, Ptgs2, Socs3, Irf8, Cd273, Cd74, MHCII, Ly6I, Xaf1, Nlrp3, Bhlhe40, Socs1, Cd86, or a group of two or more thereof.

“Irf1” as used herein, refers to the interferon regulatory factor 1 gene or the protein encoded by the gene. This gene encodes a transcription factor that regulates a wide range of genes.

“Stat1” as used herein, refers to the signal transducer and activator of transcription 1-alpha/beta gene or the protein encoded by the gene. The Stat1 protein is a transcription activator that mediates cellular response to inteferons, cytokines, and other growth factors.

“GBP2” as used herein, refers to the interferon-induced guanylate-binding protein 2 gene or a protein encoded by the gene. The GBP2 protein is a GTPas which is induced, for example, by interferon gamma.

“SlamF8” as used herein, refers to the SLAM family member 8 gene, or a protein encoded by the gene. The SlamF8 protein encodes a member of the CD2 family of cell surface proteins involved in lymphocyte activation.

“Calhm6” as used herein, refers to the calcium homeostasis modulator protein 6 gene, or a protein encoded by the gene. This gene is also known as “Fam26F”.

The genes provided herein, such as M_3 myeloid associated gene(s) are known to persons of skill in the art. Information about the genes is available at, for example, the National Center for Biotechnology Information (NCBI), the Universal Protein Resource (UniProt), The Human Protein Atlas, LifeMap Sciences (e.g. GeneCards), Catalogue Of Somatic Mutations In Cancer (COSMIC), and the National Cancer Institute (e.g. The Cancer Genome Atlas Program (TCGA)).

Exemplary sequence reference information for M_3 myeloid associated gene(s) and other genes provided herein is listed below in Table 4. Sequences (e.g. mRNA and gene) associated with the transcript accession numbers in Table 4 are available, for example at the National Center for Biotechnology Information (NCBI). While reference information for a single isoform for each gene is provide in Table 4, additional isoforms are also contemplated and within the scope of the embodiments provided herein.

TABLE 4
Transcript accession numbers of genes provided herein

| | Transcript | | Transcript |
|---|---|---|---|
| Gene | Accession No. | Gene | Accession No. |
| Irf1 | NM_002198.3 | Cd74 | NM_001025159.2 |
| Stat1 | NM_007315.4 | MHCII/ | NM_019111.5 |
| | | HLA-DRA | |
| Gbp2 | NM_004120.5 | Ly6l | NM_001368160.1 |
| Tap1 | NM_000593.6 | Cxcl10 | NM_001565.4 |
| Psmb9 | NM_002800.5 | Gbp3 | NM_018284.3 |
| Ccl5 | NM_002985.3 | Plekho1 | NM_016274.6 |
| Cd38 | NM_001775.4 | Lap3 | NM_015907.2 |
| Slamf8 | NM_020125.3 | Fcgr4 | NM_144559.2 |
| Calhm6/ | NM_001010919.3 | H2-Eb1 | NM_010382.2 |
| Fam26f | | | |
| Cd40 | NM_001250.6 | Tnfaip2 | NM_006291.4 |
| Cxcl9 | NM_002416.3 | Psme2 | NM_002818.3 |
| PD-L1/ | NM_014143.4 | Atox1 | NM_004045.4 |
| Cd274 | | | |
| Nos2 | NM_000625.4 | Gbp2b | NM_010259.2 |
| M6pr | NM_002355.4 | Ube2l6 | NM_004223.5 |
| Ly6a | NM_001271419.1 | Scimp | NM_207103.3 |
| Nfkbie | NM_004556.3 | Tapbp | NM_003190.5 |
| Trafd1 | NM_001143906.1 | Rnf19b | NM_153341.4 |
| Fcgr1 | NM_010186.5 | Pomp | NM_015932.6 |
| Dram1 | NM_018370.3 | Syngr2 | NM_004710.7 |
| IFN-gamma | NM_000619.3 | Ifi47 | NM_008330.2 |
| Tor3a | NM_022371.4 | Xaf1 | NM_017523.5 |
| Ly6c2 | NM_001357693.1 | Nlrp3 | NM_004895.5 |
| Ptgs2 | NM_000963.4 | Bhlhe40 | NM_003670.3 |
| Socs3 | NM_003955.5 | Socs1 | NM_003745.1 |
| Irf8 | NM_001363907.1 | Cd86 | NM_175862.5 |
| Cd273 | NM_025239.4 | Fancm | NM_020937.4 |
| Ren | NM_000537.4 | Gnas | NM_000516.7 |
| Wnt4 | NM_030761.5 | Wnt11 | NM_004626.3 |
| Magi2 | NM_012301.4 | Insr | NM_000208.4 |
| Itgb8 | NM_002214.3 | Arg2 | NM_001172.4 |
| Hla-F | NM_001098479.2 | Gbp1 | NM_002053.3 |
| Fanci | NM _001113378.2 | Id4 | NM_001546.4 |
| Blm | NM_000057.4 | Tigit | NM_173799.4 |
| Foxp3 | NM_014009.4 | Apc | NM_001127511.3 |
| Cdkn1a | NM_000389.5 | | |

Unless otherwise defined, all technical and scientific terms used herein have the same meaning as commonly understood by one of ordinary skill in the art to which this invention belongs. In case of conflict, the present specification, including definitions, will control. Throughout this specification and claims, the word “comprise,” or variations such as “comprises” or “comprising” will be understood to imply the inclusion of a stated integer or group of integers but not the exclusion of any other integer or group of integers. Unless otherwise required by context, singular terms shall include pluralities and plural terms shall include the singular. Throughout this specification and claims, “mg/kg” will be understood to mean mg administered per kg of body weight.

Exemplary methods and materials are described herein, although methods and materials similar or equivalent to those described herein can also be used in the practice or testing of the invention. The materials, methods, and examples are illustrative only and not intended to be limiting.

### II. Methods, Uses and Medicaments

Provided herein are biomarkers for use with PD-1 axis binding antagonist therapy, PD-1 axis binding antagonists for use with samples that have been predetermined to have certain gene mutations, gene expression profiles and/or other biomarkers, and related compositions and methods.

In one aspect of the invention, the invention provides a method, medicament or kit of parts for treating a cancer in, or improving progression free survival of, a patient comprising, for, or related to, administering to the patient a therapy which comprises a PD-1 axis binding antagonist, wherein a sample from the patient is pre-determined to have an increased expression of one or more M_3 myeloid gene(s).

The therapy may also comprise one or more therapeutic agent, such as a chemotherapeutic or chemoradio therapy.

Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as the enediyne antibiotics (e.g. calicheamicin, especially calicheamicin gamma1I and calicheamicin phiI1, see, e.g., Agnew, Chem. Intl. Ed. Engl., 33:183-186 (1994); dynemicin, including dynemicin A; bisphosphonates, such as clodronate; an esperamicin; as well as neocarzinostatin chromophore and related chromoprotein enediyne antibiotic chromomophores), aclacinomysins, actinomycin, authramycin, azaserine, bleomycins, cactinomycin, carabicin, caminomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitracrine; pentostatin; phenamet; pirarubicin; losoxantrone; podophyllinic acid; 2-ethylhydrazide; procarbazine; razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g. paclitaxel and doxetaxel; chlorambucil; gemcitabine; 6-thioguanine; mercaptopurine; methotrexate; platinum analogs such as carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; CPT-11; topoisomerase inhibitor RFS 2000; difluoromethylornithine (DMFO); retinoids such as retinoic acid; capecitabine; and pharmaceutically acceptable salts, acids or derivatives of any of the above. Also included are anti-hormonal agents that act to regulate or inhibit hormone action on tumors such as anti-estrogens and selective estrogen receptor modulators (SERMs), including, for example, tamoxifen, raloxifene, droloxifene, 4-hydroxytamoxifen, trioxifene, keoxifene, LY117018, onapristone, and toremifene (Fareston); aromatase inhibitors that inhibit the enzyme aromatase, which regulates estrogen production in the adrenal glands, such as, for example, 4(5)-imidazoles, aminoglutethimide, megestrol acetate, exemestane, formestane, fadrozole, vorozole, letrozole, and anastrozole; and anti-androgens such as flutamide, nilutamide, bicalutamide, leuprolide, and goserelin; and pharmaceutically acceptable salts, acids or derivatives of any of the above.

Each therapeutic agent in a therapy provided herein may be administered either alone or in a medicament (also referred to herein as a pharmaceutical composition) which comprises the therapeutic agent and one or more pharmaceutically acceptable carriers, excipients and diluents, according to standard pharmaceutical practice.

Each small molecule therapeutic agent in a therapy of the invention can be administered orally or parenterally, including the intravenous, intramuscular, intraperitoneal, subcutaneous, rectal, topical, and transdermal routes of administration.

A therapy of the invention may be used prior to or following surgery to remove a tumor and may be used prior to, during or after radiation therapy.

In some embodiments, a therapy of the invention is administered to a patient who has not been previously treated with a biotherapeutic or chemotherapeutic agent, i.e., is treatment-naïve. In other embodiments, the therapy is administered to a patient who failed to achieve a sustained response after prior therapy with a biotherapeutic or chemotherapeutic agent, i.e., is treatment-experienced.

A therapy of the invention is typically used to treat a tumor that is large enough to be found by palpation or by imaging techniques well known in the art, such as MRI, ultrasound, or CAT scan. In some embodiments, a therapy of the invention is used to treat an advanced stage tumor having dimensions of at least about 200 mm³, 300 mm³, 400 mm³, 500 mm³, 750 mm³, or up to 1000 mm³.

In some embodiments, a therapy of the invention is administered to a human patient who has a cancer that tests positive for PD-L1 expression. In some embodiments, PD-L1 expression can be detected using a diagnostic anti-human PD-L1 antibody, or antigen binding fragment thereof, in an IHC assay on an FFPE or frozen tissue section of a tumor sample removed from the patient. Typically, the patient's physician would order a diagnostic test to determine PD-L1 expression in a tumor tissue sample removed from the patient prior to initiation of treatment with the PD-1 axis binding antagonist, but it is envisioned that the physician could order the first or subsequent diagnostic tests at any time after initiation of treatment, such as for example after completion of a treatment cycle.

Biotherapeutic agents in a therapy of the invention may be administered by continuous infusion, or by doses at intervals of, e.g., daily, every other day, three times per week, or one time each week, two weeks, three weeks, monthly, bimonthly, etc. A total weekly dose is generally at least 0.05 μg/kg, 0.2 μg/kg, 0.5 μg/kg, 1 μg/kg, 10 μg/kg, 100 μg/kg, 0.2 mg/kg, 1.0 mg/kg, 2.0 mg/kg, 10 mg/kg, 25 mg/kg, 50 mg/kg body weight or more. See, e.g., Yang et al. (2003) New Engl. J. Med. 349:427-434; Herold et al. (2002) New Engl. J. Med. 346:1692-1698; Liu et al. (1999) J. Neurol. Neurosurg. Psych. 67:451-456; Portielji et al. (2003) Cancer Immunol. Immunother. 52:133-144.

In some embodiments that employ an anti-human PD-1 mAb or anti-human PD-L1 mAb as the PD-1 axis binding antagonist in the therapy, the dosing regimen will comprise administering the mAb at a dose of about 1, 2, 3, 5, 10, 15 or 20 mg/kg body weight, or at a dose of about 50, 80, 100, 120, 150, 180, 200, 250, 300, 400, 800, 1200 mg flat dose at intervals of about 14 days (±2 days), about 21 days (±2 days), about 28 days (±2 days), about 30 days (±2 days), about 35 days (±2 days), or about 42 days (±2 days) throughout the course of treatment.

In some embodiments that employ an anti-human PD-1 mAb or anti-human PD-1 mAb as the PD-1 axis binding antagonist in the therapy, the dosing regimen will comprise administering the mAb at a dose of from about 0.005 mg/kg to about 10 mg/kg, with intra-patient dose escalation. In other escalating dose embodiments, the interval between doses will be progressively shortened, e.g., about 30 days (±2 days) between the first and second dose, about 14 days (±2 days) between the second and third doses. In certain embodiments, the dosing interval will be about 14 days (±2 days), for doses subsequent to the second dose.

In certain embodiments, a subject will be administered an intravenous (IV) infusion of a medicament comprising any of the PD-1 axis binding antagonist described herein.

In some embodiments, the PD-1 axis binding antagonist is avelumab or a biosimilar version thereof, which is administered intravenously at a dose selected from the group consisting of: 10 mg Q2W, 10 mg Q3W, 800 mg Q2W and 1200 mg Q3W.

In some embodiments of the invention, the PD-1 axis binding antagonist is pembrolizumab (aka MK-3475) or a biosimilar version thereof, which is administered at a dose selected from the group consisting of 1 mg/kg Q2W, 2 mg/kg Q2W, 3 mg/kg Q2W, 5 mg/kg Q2W, 10 mg Q2W, 1 mg/kg Q3W, 2 mg/kg Q3W, 3 mg/kg Q3W, 5 mg/kg Q3W, 10 mg Q3W, or a flat-dose equivalents of any of these doses, i.e., such as 200 mg Q3W. In some embodiments, MK-3475 is administered at a dose of 400 mg Q6W (400 mg flat dose every six weeks). In some embodiments, MK-3475 or a biosimilar version thereof, is administered at a dose of 200 mg Q2W for adults and 2 mg/kg (up to 200 mg) Q3W for children. In some embodiments, MK-3475 is administered as a liquid medicament which comprises 25 mg/ml MK-3475, 7% (w/v) sucrose, 0.02%) (w/v) polysorbate 80 in 10 mM histidine buffer pH 5.5, and the selected dose of the medicament is administered by IV infusion over a time period of about 30 minutes. In some embodiments, MK-3475 is administered subcutaneously in a high concentration formulation, as described in U.S. Pat. No. 9,220,776, the disclosure of which is herein incorporated by reference in its entirety.

In some embodiments, the PD-1 axis binding antagonist is nivolumab or a biosimilar version thereof, which is administered at a dose of selected from the group consisting of 1 mg/kg Q2W, 2 mg/kg Q2W, 3 mg/kg Q2W, 5 mg/kg Q2W, 10 mg Q2W, 1 mg/kg Q3W, 2 mg/kg Q3W, 3 mg/kg Q3W, 5 mg/kg Q3W, and 10 mg Q3W, or a flat does equivalent of any of the forgoing doses, such as 240 mg Q2W.

In some embodiments, the PD-1 axis binding antagonist is atezolizumab or a biosimilar version thereof, which is administered at a dose of selected from the group consisting of 1 mg/kg Q2W, 2 mg/kg Q2W, 3 mg/kg Q2W, 5 mg/kg Q2W, 10 mg Q2W, 15 mg/kg Q2W 1 mg/kg Q3W, 2 mg/kg Q3W, 3 mg/kg Q3W, 5 mg/kg Q3W, and 10 mg Q3W, 15 mg/kg Q3W or a flat does equivalent of any of the forgoing doses, such as 1200 mg Q3W. in some embodiments, atezolizumab or a biosimilar version thereof is administered as an IV infusion over 60 minutes. In some embodiments, atezolizumab or a biosimilar version thereof is administered subcutaneously.

In some embodiments, the PD-1 axis binding antagonist is durvalumab or a biosimilar version thereof, which is administered at a dose of selected from the group consisting of 1 mg/kg Q2W, 2 mg/kg Q2W, 3 mg/kg Q2W, 5 mg/kg Q2W, 10 mg Q2W, 15 mg/kg Q2W 1 mg/kg Q3W, 2 mg/kg Q3W, 3 mg/kg Q3W, 5 mg/kg Q3W, and 10 mg Q3W, 15 mg/kg Q3W or a flat does equivalent of any of the forgoing doses. In some embodiments, durvalumab or a biosimilar version thereof is administered at a dose of 10 mg/kg Q2W and as an IV infusion over 60 minutes. In some embodiments, durvalumab or a biosimilar version thereof is administered subcutaneously.

In some embodiments, the PD-1 axis binding antagonist is cemiplimab or a biosimilar version thereof, which is administered at a dose of selected from the group consisting of 1 mg/kg Q2W, 2 mg/kg Q2W, 3 mg/kg Q2W, 5 mg/kg Q2W, 10 mg Q2W, 15 mg/kg Q2W 1 mg/kg Q3W, 2 mg/kg Q3W, 3 mg/kg Q3W, 5 mg/kg Q3W, and 10 mg Q3W, 15 mg/kg Q3W or a flat does equivalent of any of the forgoing doses. In some embodiments, durvalumab or a biosimilar version thereof is administered at a dose of 350 mg Q2W and as an IV infusion over 30 minutes. In some embodiments, cemiplimab or a biosimilar version thereof is administered subcutaneously.

In some embodiments, a treatment cycle begins with the first day of treatment and last for 2 weeks. In such embodiments, the therapy is preferably administered for at least 12 weeks (6 cycles of treatment), more preferably at least 24 weeks, and even more preferably at least 2 weeks after the patient achieves a CR.

The present invention also provides a medicament which comprises a PD-1 axis binding antagonist as described above and a pharmaceutically acceptable excipient. When the PD-1 binding antagonist is a biotherapeutic agent, e.g., a mAb, the antagonist may be produced in, for example, CHO or HEK cells using conventional cell culture and recovery/purification technologies.

In some embodiments, a medicament comprising an anti-PD-1 antibody or anti-PD-L1 antibody as the PD-1 axis binding antagonist may be provided as a liquid formulation or prepared by reconstituting a lyophilized powder with sterile water for injection prior to use.

The present invention also provides a medicament which comprises axitinib and a pharmaceutically acceptable excipient.

The PD-1 axis binding antagonist medicaments described herein may be provided as a kit which comprises a first container and a package insert. The first container contains at least one dose of a medicament comprising PD-1 axis binding antagonist and the package insert, or label, which comprises instructions for treating a patient for cancer using the medicaments. The kit may further comprise other materials that may be useful in administering the medicament, such as diluents, filters, IV bags and lines, needles and syringes. In some embodiments of the kit, the PD-1 axis binding antagonist is an anti-PD-L1 antibody and the instructions state that the medicaments are intended for use in treating a patient having a sample (e.g. tumor sample or tumor-associated myeloid sample) that has a gene expression profile as provided herein.

In some embodiments, in a method, medicament, PD-1 axis binding antagonist for use, or kit provided herein, the presence and/or expression level (amount) of a biomarker (e.g., M_3 myeloid associated gene(s)) is measured by determining protein expression levels of the biomarker. In certain instances, the method comprises contacting the biological sample with antibodies that specifically bind to a biomarker described herein under conditions permissive for binding of the biomarker, and detecting whether a complex is formed between the antibodies and biomarker. Such method may be an in vitro or in vivo method. Any method of measuring protein expression levels known in the art may be used. For example, in some instances, a protein expression level of a biomarker is determined using a method selected from the group consisting of flow cytometry (e.g., fluorescence-activated cell sorting (FACS)), Western blot, ELISA, ELIFA, immunoprecipitation, immunohistochemistry (IHC), immunofluorescence, radioimmunoassay, dot blotting, immunodetection methods, HPLC, surface plasmon resonance, optical spectroscopy, mass spectrometry, and HPLC. In some instances, the protein expression level of the biomarkers is determined in tumor cells.

In some embodiments, in a method, medicament, PD-1 axis binding antagonist for use, or kit provided herein, a reference level, reference sample, reference cell, reference tissue, control sample, control cell, or control tissue is a single sample or a combination of multiple samples from the same subject or individual that are obtained at one or more different time points than when the test sample is obtained. For example, a reference level, reference sample, reference cell, reference tissue, control sample, control cell, or control tissue is obtained at an earlier time point from the same subject or individual than when the test sample is obtained. Such reference level, reference sample, reference cell, reference tissue, control sample, control cell, or control tissue may be useful if the reference sample is obtained during initial diagnosis of cancer and the test sample is later obtained when the cancer becomes metastatic.

In some embodiments, in a method, medicament, PD-1 axis binding antagonist for use, or kit provided herein, a reference level, reference sample, reference cell, reference tissue, control sample, control cell, or control tissue is a combination of multiple samples from one or more healthy individuals who are not the patient.

In some embodiments, in a method, medicament, PD-1 axis binding antagonist for use, or kit provided herein, a reference level, reference sample, reference cell, reference tissue, control sample, control cell, or control tissue is a combination of multiple samples from one or more individuals with a disease or disorder (e.g., cancer) who are not the patient/individual. In certain embodiments, a reference level, reference sample, reference cell, reference tissue, control sample, control cell, or control tissue is a combination of multiple samples from multiple individuals with a disease or disorder (e.g., cancer), wherein the multiple individuals are the patient/individual plus one or more other more patients/individuals with the disease or disorder (e.g., cancer).

In some embodiments, in a method, medicament, PD-1 axis binding antagonist for use, or kit provided herein, a reference level, reference sample, reference cell, reference tissue, control sample, control cell, or control tissue is pooled RNA samples from normal tissues or pooled plasma or serum samples from one or more individuals who are not the patient.

In some embodiments, in a method, medicament, PD-1 axis binding antagonist for use, or kit provided herein, a reference level, reference sample, reference cell, reference tissue, control sample, control cell, or control tissue is pooled RNA samples from tumor tissues or pooled plasma or serum samples from one or more individuals with the disease or disorder (e.g., cancer) who are not the patient. In certain embodiments, a reference level, reference sample, reference cell, reference tissue, control sample, control cell, or control tissue is pooled RNA samples from tumor tissues or pooled plasma or serum samples from multiple individuals with the disease or disorder (e.g., cancer), wherein the multiple individuals are the patient/individual plus one or more other more patients/individuals with the disease or disorder (e.g., cancer).

In some embodiments, in a method, medicament, PD-1 axis binding antagonist for use, or kit provided herein, the reference level is the median level of expression of a biomarker across a set of samples (e.g., a set of tissue samples (e.g., a set of tumor tissue samples)). In certain embodiments, the reference level is the median level of expression of a biomarker across a population of patients having a particular disease or disorder (e.g., a proliferative cell disorder (e.g., a cancer)).

In some embodiments provided herein involving a combination of more than one sample (e.g. an average level of a biomarker across multiple samples), there are at least 3, 5, 10, 15, 20, 25, 30, 40, 50, 75, 100, 150, 200, 250, or 500 samples in the combined sample set. In some embodiments provided herein involving a combination of samples from more than one individuals/patients (e.g. an average level of a biomarker across samples from multiple individuals/patients), samples are from at least 3, 5, 10, 15, 20, 25, 30, 40, 50, 75, 100, 150, 200, 250, or 500 individuals/patients.

In some embodiments provided herein involving elevated or increased expression of a biomarker, elevated or increased expression refers to an overall increase of any of 10% or greater, 20% or greater, 30% or greater, 40% or greater, 50% or greater, 60% or greater, 70% or greater, 80% or greater, 90% or greater, 95% or greater, 96% or greater, 97% or greater, 98% or greater, 99% or greater, in the level of biomarker (e.g., protein or nucleic acid (e.g., gene or mRNA)), detected by standard art-known methods such as those described herein, as compared to a reference level, reference sample, reference cell, reference tissue, control sample, control cell, or control tissue. In certain embodiments, the elevated or increased expression refers to the increase in expression level (amount) of a biomarker in the sample, wherein the increase is at least any of 1.5 times, 1.75 times, 2 times, 3 times, 4 times, 5 times, 6 times, 7 times, 8 times, 9 times, times, 25 times, 50 times, 75 times, or 100 times the expression level (amount) of the respective biomarker in a reference level, reference sample, reference cell, reference tissue, control sample, control cell, or control tissue. In some embodiments, elevated expression refers to an overall increase of greater than 1.5-fold, 1.75-fold, 2-fold, 2.25-fold, 2.5-fold, 2.75-fold, 3.0-fold, or 3.25-fold as compared to a reference sample, reference cell, reference tissue, control sample, control cell, control tissue, or internal control (e.g., housekeeping gene).

In some embodiments, reduced expression of a biomarker, reduced or decreased expression refers to an overall reduction of any of 10% or greater, 20% or greater, 30% or greater, 40% or greater, 50% or greater, 60% or greater, 70% or greater, 80% or greater, 90% or greater, 95% or greater, 96% or greater, 97% or greater, 98% or greater, 99% or greater, in the level of biomarker (e.g., protein or nucleic acid (e.g., gene or mRNA)), detected by standard art known methods such as those described herein, as compared to a reference level, reference sample, reference cell, reference tissue, control sample, control cell, or control tissue. In certain embodiments, reduced expression refers to the decrease in expression level (amount) of a biomarker in the sample wherein the decrease is at least any of 0.9 times, 0.8 times, 0.7 times, 0.6 times, 0.5 times, 0.4 times, 0.3 times, 0.2 times, 0.1 times, 0.05 times, or 0.01 times the expression level (amount) of the respective biomarker in a reference level, reference sample, reference cell, reference tissue, control sample, control cell, control tissue, or internal control (e.g., housekeeping gene).

In some embodiments provided herein involving increased or high expression of a biomarker in a sample (e.g. gene expression), increased or high expression refers to above 0.5, 0.4, 0.3, 0.2, 0.1, 0.05, 0.04, 0.03, 0.02, or 0.01 log 2 Transcripts Per Million (TPM) in the sample.

In some embodiments provided herein involving reduced or low expression of a biomarker in a sample (e.g. gene signature), reduced or low expression refers to below 0.5, 0.4, 0.3, 0.2, 0.1, 0.05, 0.04, 0.03, 0.02, or 0.01 log 2 Transcripts Per Million (TPM) in the sample.

In embodiments that refer to a method of treatment as described herein, such embodiments are also further embodiments for use in that treatment, or alternatively for the manufacture of a medicament for use in that treatment.

These and other aspects of the invention, including the exemplary specific embodiments listed below, will be apparent from the teachings contained herein.

Exemplary embodiments provided herein included the embodiments (E) as provided below:

E1. A method of treating a patient having a cancer, comprising administering to the patient a therapeutically effective amount of a PD-1 axis binding antagonist, wherein the expression level of at least one gene selected from the group consisting of Irf1, Stat1, and Gbp2 in a sample obtained from the patient has been determined to be increased as compared to a reference level prior to treatment.
E2. The method of embodiment 1, wherein the expression level of at least two or all three of the genes selected from the group consisting of Irf1, Stat1, and Gbp2 in the sample obtained from the patient has been determined to be increased as compared to a reference level.
E3. The method of any one of embodiments 1 or 2, further wherein the expression level of at least one gene selected from the group consisting of Tap1, Psmb9, Ccl5 and Cd38 in the sample obtained from the patient has been determined to be increased as compared to a reference level.
E4. The method of embodiment 3, wherein the expression level of at least two genes selected from the group consisting of Tap1, Psmb9, Ccl5 and Cd38 in the sample obtained from the patient has been determined to be increased as compared to a reference level.
E5. The method of any one of embodiments 1-4, further wherein the expression level of at least one gene selected from the group consisting of Slamf8, Calhm6, Cd40, Cxcl9, PD-L1, Nos2, M6pr, Cd74, MHCII, and Ly6l in the sample obtained from the patient has been determined to be increased as compared to a reference level.
E6. The method of any one of embodiments 1-5, further wherein the expression level of at least one gene selected from the group consisting of Cxcl10, Tor3a, Xaf1, Nlrp3, Ptgs2, Bhlhe40, Socs3, Socs1, Irf8, Cd86, Cd273, Gbp3, Plekho1, Lap3, Tnfaip2, Psme2, Atox1, Ube2I6, Scimp, Nfkbie, Tapbp, Trafd1, Rnf19b, Pomp, Dram1, and Syngr2 in the sample obtained from the patient has been determined to be increased as compared to a reference level.
E7. A method of treating a patient having a cancer, comprising administering to the patient a therapeutically effective amount of a PD-1 axis binding antagonist, wherein the expression level of at least two, three, four, five, six, or all seven genes selected from the group consisting of Irf1, Stat1, Gbp2, Tap1, Psmb9, Ccl5 and Cd38 in a sample obtained from the patient has been determined to be increased as compared to a reference level prior to treatment.
E8. A method of treating a patient having a cancer, comprising administering to the patient a therapeutically effective amount of a PD-1 axis binding antagonist, wherein the expression level of at least one gene selected from the group consisting of Slamf8 and Calhm6 in a sample obtained from the patient has been determined to be increased as compared to a reference level prior to treatment.
E9. The method of embodiment 8, wherein the expression level of both Slamf8 and Calhm6 in the sample obtained from the patient has been determined to be increased as compared to a reference level.
E10. The method of any one of embodiments 8 or 9, further wherein the expression level of at least one gene selected from the group consisting of Cd40, Cxcl9, and PD-L1 in the sample obtained from the patient has been determined to be increased as compared to a reference level.
E11. The method of embodiment 10, wherein the expression level of at least two genes selected from the group consisting of Cd40, Cxcl9, and PD-L1 in the sample obtained from the patient has been determined to be increased as compared to a reference level.
E12. The method of any one of embodiments 8-11, further wherein the expression level of at least one gene selected from the group consisting of Nos2, Ccl5, M6pr, Cd38, Cd74, MHCII, Stat1, and Ly6l in the sample obtained from the patient has been determined to be increased as compared to a reference level.
E13. The method of any one of embodiments 8-12, further wherein the expression level of at least one gene selected from the group consisting of Cxcl10, Tor3a, Xaf1, Nlrp3, Ptgs2, Bhlhe40, Socs3, Socs1, Irf8, Cd86, Cd273, Gbp3, Plekho1, Gbp2, Lap3, Tap1, Irf1, Tnfaip2, Psme2, Atox1, Ube2I6, Scimp, Nfkbie, Psmb9, Tapbp, Trafd1, Rnf19b, Pomp, Dram1, and Syngr2 in the sample obtained from the patient has been determined to be increased as compared to a reference level.
E14. A method of treating a patient having a cancer, comprising administering to the patient a therapeutically effective amount of a PD-1 axis binding antagonist, wherein the expression level of at least two, three, four, five, six, seven, eight, nine, ten, eleven, twelve, or all thirteen genes selected from the group consisting of Slamf8, Calhm6, Cd40, Cxcl9, Nos2, Ccl5, M6pr, Cd38, Cd74, MHCII, Stat1, and Ly6l in a sample obtained from the patient has been determined to be increased as compared to a reference level prior to treatment.
E15. A method of treating a patient having a cancer, comprising administering to the patient a therapeutically effective amount of a PD-1 axis binding antagonist, wherein the expression level of the gene Cxcl9, and at least one of the genes selected from the group consisting of Irf1, Stat1, Gbp2, Slamf8, and Calhm6, in a sample obtained from the patient has been determined to be increased as compared to a reference level prior to treatment.
E16. The method of any one of embodiments 1-15, wherein the sample comprises leukocytes.
E17. The method of embodiment 16, wherein the sample further comprises at least one cell type selected from the group consisting of CD45+ cells, tumor-associated myeloid cells, and tumor-associated macrophages.
E18. The method of embodiment 16, wherein the sample consists of leukocytes and at least one cell type selected from the group consisting of CD45+ cells, tumor-associated-myeloid cells, and tumor-associated macrophages.
E19. A method of treating a patient having a cancer, comprising administering to the patient a therapeutically effective amount of a PD-1 axis binding antagonist, wherein the expression level of at least one gene selected from the group consisting of Slamf8, Calhm6, Cd40, Cxcl9, and PD-L1 in tumor-associated myeloid cells obtained from the patient has been determined to be increased as compared to a reference level.
E20. The method of embodiment 19, wherein the expression level of at least two, three, four, or all five of the genes selected from the group consisting of Slamf8, Calhm6, Cd40, Cxcl9, and PD-L1 in tumor-associated myeloid cells obtained from the patient has been determined to be increased as compared to a reference level.
E21. The method of any one of embodiments 19-20, further wherein the expression level of at least one gene selected from the group consisting of Nos2, Ccl5, M6pr, Cd38, Cd74, MHCII, Stat1, and Ly6l in tumor-associated myeloid cells obtained from the patient has been determined to be increased as compared to a reference level.
E22. The method of any one of embodiments 19-21, further wherein the expression level of at least one gene selected from the group consisting of Cxcl10, Tor3a, Xaf1, Nlrp3, Ptgs2, Bhlhe40, Socs3, Socs1, Irf8, Cd86, Cd273, Gbp3, Plekho1, Gbp2, Lap3, Tap1, Irf1, Tnfaip2, Psme2, Atox1, Ube2I6, Scimp, Nfkbie, Psmb9, Tapbp, Trafd1, Rnf19b, Pomp, Dram1, and Syngr2 in tumor-associated myeloid cells obtained from the patient has been determined to be increased as compared to a reference level.
E23. A method of identifying a patient having a cancer who may benefit from a treatment comprising a therapeutically effective amount of a PD-1 axis binding antagonist, the method comprising determining an expression level of at least one, two, or all three genes selected from the group consisting of Irf1, Stat1, and Gbp2 in a sample obtained from the patient, wherein an increased expression level of the at least one, two, or all three genes in the sample as compared to a reference level identifies the patient as one who has an increased likelihood of benefiting from a treatment comprising a therapeutically effective amount of a PD-1 axis binding antagonist.
E24. A method of predicting responsiveness of a patient having a cancer to a treatment comprising a therapeutically effective amount of a PD-1 axis binding antagonist, the method comprising determining an expression level of at least one, two, or all three genes selected from the group consisting of Irf1, Stat1, and Gbp2 in a sample obtained from the patient, wherein an increased expression level of the at least one, two, or all three genes in the sample as compared to a reference level indicates that the patient has an increased likelihood of benefiting from a treatment comprising a therapeutically effective amount of a PD-1 axis binding antagonist.
E25. A medicament comprising a PD-1 axis binding antagonist for use in treating a cancer in a patient, wherein a sample from the patient is pre-determined to have at least one of and optionally two, three, four, five or all of the following characteristics:
(i) it has an increased expression level of at least one, two, or all three of the genes Irf1, Stat1, and Gbp2 as compared to a reference level;
(ii) it has an increased expression level of at least 1, 2, 3, 4, 5, 6 or all 7 genes selected from the group consisting of Irf1, Stat1, Gbp2, Tap1, Psmb9, Ccl5 and Cd38 as compared to a reference level;
(iii) it has an increased expression level of at least 1, 2, 3, 4, 5, 6, 7, 8, 10, 15, or all 18 genes selected from the group consisting of Irf1, Stat1, Gbp2, Tap1, Psmb9, Ccl5, Cd38, Slamf8, Calhm6, Cd40, Cxcl9, PD-L1, Nos2, M6pr, Cd74, MHCII, and Ly6l as compared to a reference level;
(iv) it has an increased expression level of at least 1, 2, 3, 4, 5, 6, 7, 8, 10, 15, 20, or 25 genes selected from the group consisting of Irf1, Stat1, Gbp2, Tap1, Psmb9, Ccl5, Cd38, Slamf8, Calhm6, Cd40, Cxcl9, PD-L1, Nos2, M6pr, Cd74, MHCII, Ly6I, Cxcl10, Tor3a, Xaf1, Nlrp3, Ptgs2, Bhlhe40, Socs3, Socs1, Irf8, Cd86, Cd273, Gbp3, Plekho1, Lap3, Tnfaip2, Psme2, Atox1, Ube2I6, Scimp, Nfkbie, Tapbp, Trafd1, Rnf19b, Pomp, Dram1, and Syngr2 i as compared to a reference level;
(v) the sample comprises or consists of leukocytes, CD45+ cells, myeloid cells, or tumor-associated myeloid cells;
(vi) it has an increased expression level of one or both of the genes Slamf8 and Calhm6 as compared to a reference level.
   E26. A kit which comprises a first container and a package insert, wherein the first container comprises at least one dose of a medicament comprising an PD-1 axis binding antagonist and the package insert comprises instructions for treating a subject for cancer wherein a sample from the patient is pre-determined as having at least one of and optionally two, three, four, five or all of the following characteristics:
(i) it has an increased expression level of at least one, two, or all three of the genes Irf1, Stat1, and Gbp2 as compared to a reference level;
(ii) it has an increased expression level of at least 1, 2, 3, 4, 5, 6 or all 7 genes selected from the group consisting of Irf1, Stat1, Gbp2, Tap1, Psmb9, Ccl5 and Cd38 as compared to a reference level;
(iii) it has an increased expression level of at least 1, 2, 3, 4, 5, 6, 7, 8, 10, 15, or all 18 genes selected from the group consisting of Irf1, Stat1, Gbp2, Tap1, Psmb9, Ccl5, Cd38, Slamf8, Calhm6, Cd40, Cxcl9, PD-L1, Nos2, M6pr, Cd74, MHCII, and Ly6l as compared to a reference level;
(iv) it has an increased expression level of at least 1, 2, 3, 4, 5, 6, 7, 8, 10, 15, 20, or 25 genes selected from the group consisting of Irf1, Stat1, Gbp2, Tap1, Psmb9, Ccl5, Cd38, Slamf8, Calhm6, Cd40, Cxcl9, PD-L1, Nos2, M6pr, Cd74, MHCII, Ly6I, Cxcl10, Tor3a, Xaf1, Nlrp3, Ptgs2, Bhlhe40, Socs3, Socs1, Irf8, Cd86, Cd273, Gbp3, Plekho1, Lap3, Tnfaip2, Psme2, Atox1, Ube2I6, Scimp, Nfkbie, Tapbp, Trafd1, Rnf19b, Pomp, Dram1, and Syngr2 as compared to a reference level;
(v) the sample comprises or consists of leukocytes, CD45+ cells, myeloid cells, or tumor-associated myeloid cells;
(vi) it has an increased expression level of one or both of the genes Slamf8 and Calhm6 as compared to a reference level.
   E27. A PD-1 axis binding antagonist for use in a method of treating a cancer in a patient, wherein the method comprises:
a. determining whether a sample from the patient has at least one and optionally two, three, four, five or all of the following characteristics:
   (i) it has an increased expression level of at least one, two, or all three of the genes Irf1, Stat1, and Gbp2 as compared to a reference level;
   (ii) it has an increased expression level of at least 1, 2, 3, 4, 5, 6 or all 7 genes selected from the group consisting of Irf1, Stat1, Gbp2, Tap1, Psmb9, Ccl5 and Cd38 as compared to a reference level;
   (iii) it has an increased expression level of at least 1, 2, 3, 4, 5, 6, 7, 8, 10, 15, or all 18 genes selected from the group consisting of Irf1, Stat1, Gbp2, Tap1, Psmb9, Ccl5, Cd38, Slamf8, Calhm6, Cd40, Cxcl9, PD-L1, Nos2, M6pr, Cd74, MHCII, and Ly6l as compared to a reference level;
   (iv) it has an increased expression level of at least 1, 2, 3, 4, 5, 6, 7, 8, 10, 15, 20, or 25 genes selected from the group consisting of Irf1, Stat1, Gbp2, Tap1, Psmb9, Ccl5, Cd38, Slamf8, Calhm6, Cd40, Cxcl9, PD-L1, Nos2, M6pr, Cd74, MHCII, Ly6I, Cxcl10, Tor3a, Xaf1, Nlrp3, Ptgs2, Bhlhe40, Socs3, Socs1, Irf8, Cd86, Cd273, Gbp3, Plekho1, Lap3, Tnfaip2, Psme2, Atox1, Ube2I6, Scimp, Nfkbie, Tapbp, Trafd1, Rnf19b, Pomp, Dram1, and Syngr2 i as compared to a reference level;
   (v) the sample comprises or consists of leukocytes, CD45+ cells, myeloid cells, or tumor-associated macrophages;
   (vi) it has an increased expression level of one or both of the genes Slamf8 and Calhm6 as compared to a reference level, and
b. if the sample has said characteristic(s), administering to the patient an effective amount of the PD-1 axis binding antagonist.
   E28. Use of a PD-1 axis binding antagonist for the manufacture of a medicament for the treatment of cancer in a patient, wherein a sample from the patient is pre-determined to have at least one of and optionally two, three, four, five, or all of the following characteristics:
(i) it has an increased expression level of at least one, two, or all three of the genes Irf1, Stat1, and Gbp2 as compared to a reference level;
(ii) it has an increased expression level of at least 1, 2, 3, 4, 5, 6 or all 7 genes selected from the group consisting of Irf1, Stat1, Gbp2, Tap1, Psmb9, Ccl5 and Cd38 as compared to a reference level;
(iii) it has an increased expression level of at least 1, 2, 3, 4, 5, 6, 7, 8, 10, 15, or all 18 genes selected from the group consisting of Irf1, Stat1, Gbp2, Tap1, Psmb9, Ccl5, Cd38, Slamf8, Calhm6, Cd40, Cxcl9, PD-L1, Nos2, M6pr, Cd74, MHCII, and Ly6l as compared to a reference level;
(iv) it has an increased expression level of at least 1, 2, 3, 4, 5, 6, 7, 8, 10, 15, 20, or 25 genes selected from the group consisting of Irf1, Stat1, Gbp2, Tap1, Psmb9, Ccl5, Cd38, Slamf8, Calhm6, Cd40, Cxcl9, PD-L1, Nos2, M6pr, Cd74, MHCII, Ly6I, Cxcl10, Tor3a, Xaf1, Nlrp3, Ptgs2, Bhlhe40, Socs3, Socs1, Irf8, Cd86, Cd273, Gbp3, Plekho1, Lap3, Tnfaip2, Psme2, Atox1, Ube2I6, Scimp, Nfkbie, Tapbp, Trafd1, Rnf19b, Pomp, Dram1, and Syngr2 i as compared to a reference level;
(v) the sample comprises or consists of leukocytes, CD45+ cells, myeloid cells, or tumor-associated macrophages;
(vi) it has an increased expression level of one or both of the genes Slamf8 and Calhm6 as compared to a reference level.
   E29. A method of treating a patient having a cancer, comprising administering to the patient a therapeutically effective amount of a PD-1 axis binding antagonist, wherein a sample from the patient is pre-determined to have at least one of and optionally two, three, four, five, or all of the following characteristics:
(i) it has an increased expression level of at least one, two, or all three of the genes Irf1, Stat1, and Gbp2 as compared to a reference level;
(ii) it has an increased expression level of at least 1, 2, 3, 4, 5, 6 or all 7 genes selected from the group consisting of Irf1, Stat1, Gbp2, Tap1, Psmb9, Ccl5 and Cd38 as compared to a reference level;
(iii) it has an increased expression level of at least 1, 2, 3, 4, 5, 6, 7, 8, 10, 15, or all 18 genes selected from the group consisting of Irf1, Stat1, Gbp2, Tap1, Psmb9, Ccl5, Cd38, Slamf8, Calhm6, Cd40, Cxcl9, PD-L1, Nos2, M6pr, Cd74, MHCII, and Ly6l as compared to a reference level;
(iv) it has an increased expression level of at least 1, 2, 3, 4, 5, 6, 7, 8, 10, 15, 20, or 25 genes selected from the group consisting of Irf1, Stat1, Gbp2, Tap1, Psmb9, Ccl5, Cd38, Slamf8, Calhm6, Cd40, Cxcl9, PD-L1, Nos2, M6pr, Cd74, MHCII, Ly6I, Cxcl10, Tor3a, Xaf1, Nlrp3, Ptgs2, Bhlhe40, Socs3, Socs1, Irf8, Cd86, Cd273, Gbp3, Plekho1, Lap3, Tnfaip2, Psme2, Atox1, Ube2I6, Scimp, Nfkbie, Tapbp, Trafd1, Rnf19b, Pomp, Dram1, and Syngr2 i as compared to a reference level;
(v) the sample comprises or consists of leukocytes, CD45+ cells, myeloid cells, or tumor-associated macrophages;
(vi) it has an increased expression level of one or both of the genes Slamf8 and Calhm6 as compared to a reference level.
   E30. A method of treating a patient having a cancer, comprising administering to the patient a therapeutically effective amount of a PD-1 axis binding antagonist, wherein a sample from the patient is pre-determined to have at least one of and optionally two or all three of the following characteristics:
(i) it has one or both of the following characteristics: a) a protein altering mutation in at least one, two, or all three genes selected from the group consisting of Fanci, Fancm, and Blm; b) an increased tumor mutational burden (TMB) as compared to a reference level;
(ii) it has at least one, two, or all three of the following characteristics: a) an increased expression level of at least 1, 2, 3, 4, 5, 6 or all 7 genes selected from the group consisting of Cxcl9, Ifng, Cxcl10, FoxP3, Tigit, Gbp1, Hla-F as compared to a reference level; b) a decreased expression level of the gene Arg2 as compared to a reference level; c) a wild-type version of the gene Gnas;
(iii) it has at least one, two, or all three of the following characteristics: a) an increased expression level of at least one, two, or all three genes selected from the group consisting of Wnt11, Id4, CDKN1A as compared to a reference level; b) a decreased expression level of at least one, two, or all three genes selected from the group consisting of Ren, Wnt4, and Itgb8 as compared to a reference level; c) a protein altering mutation in at least one, two, or all three genes selected from the group consisting of Insr, Apc, and Magi2.
   E31. A method of treating a patient having a cancer, comprising administering to the patient a therapeutically effective amount of a PD-1 axis binding antagonist, wherein a sample from the patient is pre-determined to have at least 6 and optionally 7, 8, 9, 10, 11, 12, 13, 14, 15, 16, 17, 18, 19, 20, 21, or all 22 of the following characteristics:
(i) a protein altering mutation in the gene Fanci;
(ii) a protein altering mutation in the gene Fancm;
(iii) a protein altering mutation in the gene Blm;
(iv) an increased tumor mutational burden (TMB) as compared to a reference level;
(v) increased expression level of the gene Cxcl9 as compared to a reference level;
(vi) increased expression level of the gene Ifng as compared to a reference level;
(vii) increased expression level of the gene Cxcl10 as compared to a reference level;
(viii) increased expression level of the gene Foxp3 as compared to a reference level;
(ix) increased expression level of the gene Tigit as compared to a reference level;
(x) increased expression level of the gene Gbp1 as compared to a reference level;
(xi) increased expression level of the gene Hla-F as compared to a reference level;
(xii) decreased expression level of the gene Arg2 as compared to a reference level;
(xiii) wild-type version of the gene Gnas;
(xiv) increased expression level of the gene Wnt11 as compared to a
(xv) increased expression level of the gene Id4 as compared to a reference level;
(xvi) increased expression level of the gene Cdkn1a as compared to a reference level;
(xvii) decreased expression level of the gene Ren as compared to a reference level;
(xviii) decreased expression level of the gene Wnt4 as compared to a reference level;
(xix) decreased expression level of the gene Itgb8 as compared to a reference level;
(xx) a protein altering mutation in the gene Insr;
(xxi) a protein altering mutation in the gene Apc;
(xxii) a protein altering mutation in the gene Magi2.
   E32. The method of embodiment 30 or 31, wherein the sample is pre-determined to have at least 3 and optionally 4, 5, 6, 7, 8, 9, 10, 11, 12, 13, 14, 15, or all 16 of the following characteristics:
(i) a protein altering mutation in the gene Fanci;
(ii) a protein altering mutation in the gene Fancm;
(iii) a protein altering mutation in the gene Blm;
(iv) increased expression level of the gene Gbp1 as compared to a reference level;
(v) increased expression level of the gene Hla-F as compared to a reference level;
(vi) decreased expression level of the gene Arg2 as compared to a reference level;
(vii) wild-type version of the gene Gnas;
(viii) increased expression level of the gene Wnt11 as compared to a reference level;
(ix) increased expression level of the gene Id4 as compared to a reference level;
(x) increased expression level of the gene Cdkn1a as compared to a reference level;
(xi) decreased expression level of the gene Ren as compared to a
(xii) decreased expression level of the gene Wnt4 as compared to a reference level;
(xiii) decreased expression level of the gene Itgb8 as compared to a reference level;
(xiv) a protein altering mutation in the gene Insr;
(xv) a protein altering mutation in the gene Apc;
(xvi) a protein altering mutation in the gene Magi2.
   E33. The method of any one of embodiments 30-32, wherein the sample is pre-determined to have the following characteristics:
(i) a protein altering mutation in the gene Fanci;
(ii) a protein altering mutation in the gene Fancm;
(iii) a protein altering mutation in the gene Blm;
(iv) an increased tumor mutational burden (TMB) as compared to a reference level;
(v) increased expression level of the gene Cxcl9 as compared to a reference level;
(vi) increased expression level of the gene Ifng as compared to a reference level;
(vii) increased expression level of the gene Cxcl10 as compared to a reference level;
(viii) increased expression level of the gene Foxp3 as compared to a reference level;
(ix) increased expression level of the gene Tigit as compared to a reference level;
(x) increased expression level of the gene Gbp1 as compared to a reference level;
(xi) increased expression level of the gene Hla-F as compared to a reference level;
(xii) decreased expression level of the gene Arg2 as compared to a reference level;
(xiii) wild-type version of the gene Gnas;
(xiv) increased expression level of the gene Wnt11 as compared to a reference level;
(xv) increased expression level of the gene Id4 as compared to a
(xvi) increased expression level of the gene Cdkn1a as compared to a reference level;
(xvii) decreased expression level of the gene Ren as compared to a reference level;
(xviii) decreased expression level of the gene Wnt4 as compared to a reference level;
(xix) decreased expression level of the gene Itgb8 as compared to a reference level;
(xx) a protein altering mutation in the gene Insr;
(xxi) a protein altering mutation in the gene Apc;
(xxii) a protein altering mutation in the gene Magi2.
   E34. Use of a PD-1 axis binding antagonist for the manufacture of a medicament for the treatment of cancer in a patient according to any one of embodiments 30-33.
   E35. A method for treating cancer in a patient, wherein the cancer in the patient is pre-determined to contain one or more protein altering mutations in one or more gene(s) selected from the group consisting of FRYL, FER1L5, NBEA, SACS, CEP350, SOX17, SSPO, TENM4, KCP, ARAP1, NUP160, PTPRS, C6ORF132, KIAA1551, EPB41, MYH4, TRIM10, and TOP2B and/or to not contain a protein altering mutation in one or more gene(s) selected from the group consisting of DNAH1, AGRN, NHSL1, EPHB4, EYS, GNAS, PLXNC1, AKAP1, and TBCD; the method comprising administering to the patient a therapeutically effective amount of a PD-1 axis binding antagonist. Optionally, the cancer is urothelial carcinoma. Optionally the method further comprises administering to the patient BSC for urothelial carcinoma, as described in Example 4 herein.
   E36. A method for treating cancer in a patient, the method comprising identifying if the cancer in the patient contains one or more protein altering mutations in one or more gene(s) selected from the group consisting of FRYL, FER1 L5, NBEA, SACS, CEP350, SOX17, SSPO, TENM4, KCP, ARAP1, NUP160, PTPRS, C6ORF132, KIAA1551, EPB41, MYH4, TRIM10, and TOP2B, and if the cancer contains one or more protein altering mutations in one or more the genes, administering to the patient a therapeutically effective amount of a PD-1 axis binding antagonist. Optionally, the cancer is urothelial carcinoma. Optionally the method further comprises administering to the patient BSC for urothelial carcinoma, as described in Example 4 herein.
   E37. A method for treating cancer in a patient, the method comprising identifying if the cancer in the patient does not contain one or more protein altering mutations in one or more gene(s) selected from the group consisting of DNAH1, AGRN, NHSL1, EPHB4, EYS, GNAS, PLXNC1, AKAP1, and TBCD, and if the cancer does not contain one or more protein altering mutations in one or more the genes, administering to the patient a therapeutically effective amount of a PD-1 axis binding antagonist. Optionally, the cancer is urothelial carcinoma. Optionally the method further comprises administering to the patient BSC for urothelial carcinoma, as described in Example 4 herein.
   E38. Use of a PD-1 axis binding antagonist for the manufacture of a medicament for the treatment of cancer in a patient according to any one of embodiments 35-37.
   E39. The method, medicament, PD-1 axis binding antagonist, use, or kit of any one of embodiments 1-38, wherein the respective reference level of gene expression or TMB is determined based on an average level of the gene expression or TMB from a plurality of samples from patients having the cancer.
   E40. The method, medicament, or kit of any one of embodiments 1-38, wherein the respective reference level of gene expression or TMB is determined based on an average level of the gene expression or TMB from a plurality of samples from human subjects.
   E41. The method, medicament, PD-1 axis binding antagonist, use, or kit of any one of embodiments 1-38, wherein the respective reference level of gene expression is the level of gene expression of a reference gene in a cancer from the patient.
   E42. The method, medicament, PD-1 axis binding antagonist, use, or kit of any one of embodiments 1-41, wherein the sample obtained from the patient is a tissue sample, a whole blood sample, a plasma sample, or a serum sample.
   E43. The method, medicament, PD-1 axis binding antagonist, use, or kit of embodiment 42, wherein the tissue sample is a tumor tissue sample.
   E44. The method, medicament, PD-1 axis binding antagonist, use, or kit of any one of embodiments 1-41, wherein the sample comprises or consists of tumor associated leukocytes, tumor associated CD45+ cells, tumor associated myeloid cells, or tumor associated macrophages.
   E45. The method, medicament, PD-1 axis binding antagonist, use, or kit of any one of embodiments 1-44, wherein the expression level is an mRNA expression level.
   E46. The method, medicament, PD-1 axis binding antagonist, use, or kit of embodiment 45, wherein the mRNA expression level is determined by RNA sequencing, RT-PCR, gene expression profiling, serial analysis of gene expression, or microarray analysis.
   E47. The method, medicament, PD-1 axis binding antagonist, use, or kit of any one of embodiments 1-44, wherein the expression level is a protein expression level.
   E48. The method, medicament, PD-1 axis binding antagonist, use, or kit of any one of embodiments 1-47, wherein the PD-1 axis binding antagonist is an anti-PD-1 antibody.
   E49. The method, medicament, PD-1 axis binding antagonist, use, or kit of embodiment 48, wherein the anti-PD-1 antibody is selected from the group consisting of pembrolizumab, nivolumab, cemiplimab and RN888.
   E50. The method, medicament, PD-1 axis binding antagonist, use, or kit of embodiment 49, wherein the anti-PD-1 antibody comprises
(a) a full length heavy chain having an amino acid sequence of SEQ ID NO: 9, and a full length light chain having an amino acid sequence of SEQ ID NO: 10;
(b) a full length heavy chain having an amino acid sequence of SEQ ID NO: 7, and a full length light chain having an amino acid sequence of SEQ ID NO:8; or
(c) a heavy chain variable region (VH) having an amino acid sequence of SEQ ID NO:5, and a light chain variable region (VL) of an amino acid sequence of SEQ ID NO:4.
   E51. The method, medicament, PD-1 axis binding antagonist, use, or kit of any one of embodiments 1-47, wherein the PD-1 axis binding antagonist is an anti-PD-L1 antibody.
   E52. The method, medicament, PD-1 axis binding antagonist, use, or kit of embodiment 51, wherein the anti-PD-L1 antibody is selected from the group consisting of avelumab, atezolizumab and durvalumab.
   E53. The method, medicament, PD-1 axis binding antagonist, use, or kit of embodiment 52, wherein the anti-PD-L1 antibody comprises
(a) a VH having an amino acid sequence of SEQ ID NO:14, and a VL having an amino acid sequences of SEQ ID NO:15; or
(b) a VH having an amino acid sequence of SEQ ID NO:12, and a VL having an amino acid sequence of SEQ ID NO:13.
   E54. The method, medicament, PD-1 axis binding antagonist, use, or kit of any one of embodiments 1-53, wherein the PD-1 axis binding antagonist is administered at a dose of about 5 mg/kg, about 10 mg/kg, about 200 mg, about 240 mg, about 800 mg or about 1200 mg, and is administered about once a week, or about once every two, three, four, five weeks or six weeks.
   E55. The method, medicament, PD-1 axis binding antagonist, use, or kit of any one of embodiments 1-54, wherein the PD-1 axis binding antagonist is administered with at least a second anti-cancer therapeutic.
   E56. The method, medicament, PD-1 axis binding antagonist, use, or kit of any one of embodiments 1-55, wherein the cancer is advanced or metastatic solid tumor.
   E57. The method, medicament, PD-1 axis binding antagonist, use, or kit of any one of embodiments 1-56, wherein the cancer is bladder cancer, breast cancer, clear cell kidney cancer, lung squamous cell carcinoma, malignant melanoma, non-small-cell lung cancer (NSCLC), ovarian cancer, pancreatic cancer, prostate cancer, renal cell carcinoma, small-cell lung cancer (SCLC), triple negative breast cancer, acute lymphoblastic leukemia (ALL), acute myeloid leukemia (AML), chronic lymphocytic leukemia (CLL), chronic myeloid leukemia (CML), diffuse large B-cell lymphoma (DLBCL), follicular lymphoma, Hodgkin's lymphoma (HL), liver cancer, mantle cell lymphoma (MCL), multiple myeloma (MM), myelodysplastic syndrome (MDS), non-Hodgkin's lymphoma (NHL), Squamous Cell Carcinoma of the Head and Neck (SCCHN), small lymphocytic lymphoma (SLL), endometrial cancer, B-cell acute lymphoblastic leukemia, colorectal cancer, glioblastoma, cervical cancer, penile cancer, or non-melanoma skin cancer.

### III. General Methods

Standard methods in molecular biology are described Sambrook, Fritsch and Maniatis (1982 & 1989 2nd Edition, 2001 3rd Edition) Molecular Cloning, A Laboratory Manual, Cold Spring Harbor Laboratory Press, Cold Spring Harbor, N.Y.; Sambrook and Russell (2001) Molecular Cloning, 3rd ed., Cold Spring Harbor Laboratory Press, Cold Spring Harbor, N.Y.; Wu (1993) Recombinant DNA, Vol. 217, Academic Press, San Diego, Calif.). Standard methods also appear in Ausbel, et al. (2001) Current Protocols in Molecular Biology, Vols. 1-4, John Wiley and Sons, Inc. New York, N.Y., which describes cloning in bacterial cells and DNA mutagenesis (Vol. 1), cloning in mammalian cells and yeast (Vol. 2), glycoconjugates and protein expression (Vol. 3), and bioinformatics (Vol. 4).

Methods for protein purification including immunoprecipitation, chromatography, electrophoresis, centrifugation, and crystallization are described (Coligan, et al. (2000) Current Protocols in Protein Science, Vol. 1, John Wiley and Sons, Inc., New York). Chemical analysis, chemical modification, post-translational modification, production of fusion proteins, glycosylation of proteins are described (see, e.g., Coligan, et al. (2000) Current Protocols in Protein Science, Vol. 2, John Wiley and Sons, Inc., New York; Ausubel, et al. (2001) Current Protocols in Molecular Biology, Vol. 3, John Wiley and Sons, Inc., NY, NY, pp. 16.0.5-16.22.17; Sigma-Aldrich, Co. (2001) Products for Life Science Research, St. Louis, Mo.; pp. 45-89; Amersham Pharmacia Biotech (2001) BioDirectory, Piscataway, N.J., pp. 384-391). Production, purification, and fragmentation of polyclonal and monoclonal antibodies are described (Coligan, et al. (2001) Current Protcols in Immunology, Vol. 1, John Wiley and Sons, Inc., New York; Harlow and Lane (1999) Using Antibodies, Cold Spring Harbor Laboratory Press, Cold Spring Harbor, N.Y.; Harlow and Lane, supra). Standard techniques for characterizing ligand/receptor interactions are available (see, e.g., Coligan, et al. (2001) Current Protocols in Immunology, Vol. 4, John Wiley, Inc., New York).

Monoclonal, polyclonal, and humanized antibodies can be prepared (see, e.g., Sheperd and Dean (eds.) (2000) Monoclonal Antibodies, Oxford Univ. Press, New York, N.Y.; Kontermann and Dubel (eds.) (2001) Antibody Engineering, Springer-Verlag, New York; Harlow and Lane (1988) Antibodies A Laboratory Manual, Cold Spring Harbor Laboratory Press, Cold Spring Harbor, N.Y., pp. 139-243; Carpenter, et al. (2000) J. Immunol. 165:6205; He, et al. (1998) J. Immunol. 160:1029; Tang et al. (1999) J. Biol. Chem. 274:27371-27378; Baca et al. (1997) J. Biol. Chem. 272:10678-10684; Chothia et al. (1989) Nature 342:877-883; Foote and Winter (1992) J. Mol. Biol. 224:487-499; U.S. Pat. No. 6,329,511).

An alternative to humanization is to use human antibody libraries displayed on phage or human antibody libraries in transgenic mice (Vaughan et al. (1996) Nature Biotechnol. 14:309-314; Barbas (1995) Nature Medicine 1:837-839; Mendez et al. (1997) Nature Genetics 15:146-156; Hoogenboom and Chames (2000) Immunol. Today 21:371-377; Barbas et al. (2001) Phage Display: A Laboratory Manual, Cold Spring Harbor Laboratory Press, Cold Spring Harbor, N.Y.; Kay et al. (1996) Phage Display of Peptides and Proteins: A Laboratory Manual, Academic Press, San Diego, Calif.; de Bruin et al. (1999) Nature Biotechnol. 17:397-399).

Purification of antigen is not necessary for the generation of antibodies. Animals can be immunized with cells bearing the antigen of interest. Splenocytes can then be isolated from the immunized animals, and the splenocytes can fused with a myeloma cell line to produce a hybridoma (see, e.g., Meyaard et al. (1997) Immunity 7:283-290; Wright et al. (2000) Immunity 13:233-242; Preston et al., supra; Kaithamana et al. (1999) J. Immunol. 163:5157-5164).

Antibodies can be conjugated, e.g., to small drug molecules, enzymes, liposomes, polyethylene glycol (PEG). Antibodies are useful for therapeutic, diagnostic, kit or other purposes, and include antibodies coupled, e.g., to dyes, radioisotopes, enzymes, or metals, e.g., colloidal gold (see, e.g., Le Doussal et al. (1991) J. Immunol. 146:169-175; Gibellini et al. (1998) J. Immunol. 160:3891-3898; Hsing and Bishop (1999) J. Immunol. 162:2804-2811; Everts et al. (2002) J. Immunol. 168:883-889).

Methods for flow cytometry, including fluorescence activated cell sorting (FACS), are available (see, e.g., Owens, et al. (1994) Flow Cytometry Principles for Clinical Laboratory Practice, John Wiley and Sons, Hoboken, N.J.; Givan (2001) Flow Cytometry, 2nd ed.; Wiley-Liss, Hoboken, N.J.; Shapiro (2003) Practical Flow Cytometry, John Wiley and Sons, Hoboken, N.J.). Fluorescent reagents suitable for modifying nucleic acids, including nucleic acid primers and probes, polypeptides, and antibodies, for use, e.g., as diagnostic reagents, are available (Molecular Probesy (2003) Catalogue, Molecular Probes, Inc., Eugene, Oreg.; Sigma-Aldrich (2003) Catalogue, St. Louis, Mo.).

Standard methods of histology of the immune system are described (see, e.g., Muller-Harmelink (ed.) (1986) Human Thymus: Histopathology and Pathology, Springer Verlag, New York, N.Y.; Hiatt, et al. (2000) Color Atlas of Histology, Lippincott, Williams, and Wilkins, Phila, Pa.; Louis, et al. (2002) Basic Histology: Text and Atlas, McGraw-Hill, New York, N.Y.).

Software packages and databases for determining, e.g., antigenic fragments, leader sequences, protein folding, functional domains, glycosylation sites, and sequence alignments, are available (see, e.g., GenBank, Vector NTI® Suite (Informax, Inc, Bethesda, Md.); GCG Wisconsin Package (Accelrys, Inc., San Diego, Calif.); DeCypher® (TimeLogic Corp., Crystal Bay, Nevada); Menne, et al. (2000) Bioinformatics 16: 741-742; Menne, et al. (2000) Bioinformatics Applications Note 16:741-742; Wren, et al. (2002) Comput. Methods Programs Biomed. 68:177-181; von Heijne (1983) Eur. J. Biochem. 133:17-21; von Heijne (1986) Nucleic Acids Res. 14:4683-4690).

The presence and/or expression level (amount) of various biomarkers described herein in a sample can be analyzed by a number of methodologies, many of which are known in the art and understood by the skilled artisan, including, but not limited to, immunohistochemistry (“IHC”), Western blot analysis, immunoprecipitation, molecular binding assays, enzyme-linked immunosorbent assay (ELISA), enzyme-linked immunofiltration assay (ELIFA), fluorescence activated cell sorting (“FACS”), MassARRAY, proteomics, quantitative blood based assays (e.g., serum ELISA), biochemical enzymatic activity assays, in situ hybridization, fluorescence in situ hybridization (FISH), Southern analysis, Northern analysis, whole genome sequencing, polymerase chain reaction (PCR) (including quantitative real time PCR (qRT-PCR) and other amplification type detection methods, such as, for example, branched DNA, SISBA, TMA and the like), RNA-Seq, microarray analysis, gene expression profiling, and/or serial analysis of gene expression (“SAGE”), as well as any one of the wide variety of assays that can be performed by protein, gene, and/or tissue array analysis. Typical protocols for evaluating the status of genes and gene products are found, for example in Ausubel et al., eds., 1995, Current Protocols In Molecular Biology, Units 2 (Northern Blotting), 4 (Southern Blotting), 15 (Immunoblotting) and 18 (PCR Analysis). Multiplexed immunoassays such as those available from Rules Based Medicine or Meso Scale Discovery (“MSD”) may also be used.

Incorporated by reference herein for all purposes is the content of U.S. Provisional Patent Application Nos. 63/046,957 (filed Jul. 1, 2020), 62/706,712 (filed Sep. 4, 2020), 62/706,912 (filed Sep. 17, 2020) and 63/193,306 (filed May 26, 2021).

### IV. Examples

### Example 1: Identification of a Tumor-Associated Myeloid Subset Associated with Response to PD-1 Axis Antagonist Therapy

This Example describes the identification of a tumor-associated myeloid cell subset with anti-tumor functions whose presence is predictive of successful PD-1 axis antagonist therapy.

### Background

Successful ICT elicits effective antitumor responses dependent on CD8 T cells; however, the suppressive tumor microenvironment can effectively mute these responses (Sharma and Allison, 2015). Tumor-associated myeloid cells, most notably tumor-associated macrophages (TAMs), are among the most abundant constituents of the tumor microenvironment (Qian and Pollard, 2010), and their presence is associated not only with poor prognosis but also with resistance to ICT (Cassetta and Kitamura, 2018). Recent single-cell RNA sequencing (scRNA-Seq) and mass cytometry analyses of tumor-infiltrating immune populations have unveiled considerable and unexpected heterogeneity within the TAM compartment (Chevrier et al., 2017; Zilionis et al., 2019). Currently little is known about the roles of individual TAM subsets; however, there is evidence that some possess detrimental functions. For instance, a mass cytometry study of patients with clear cell renal cell carcinoma identified an association between a TAM subset expressing CCL8 and PD-L1, T-cell exhaustion, and poor survival (Chevrier et al., 2017). Additionally, the abundance of a preexisting MHCII^{low} myeloid phenotype identified by scRNA-Seq in patients with metastatic melanoma predicted a poor response to ICT (Krieg et al., 2018). As TAMs have generally been considered bad actors, therapeutic strategies aimed at modulating these cells have attracted considerable interest, with ongoing clinical research programs aiming to deplete TAMs or otherwise influence their function via recruitment or repolarization (Cassetta and Kitamura, 2018). However, clinical trials exploring TAM depletion have yielded little benefit to date (Peyraud et al., 2017), potentially because broad depletion strategies may affect both beneficial and immunosuppressive TAM subsets.

### A Bilateral Tumor Model to Identify Biomarkers of Response to Anti-PD-L1 Treatment

To determine whether preexisting tumor immune populations shape the response to treatment with avelumab syngeneic tumor models were screened to identify models that elicit a partial response to treatment. Only CT26 colorectal carcinoma was determined to exhibit such heterogeneity, demonstrating a roughly 50% response rate. In contrast, mice bearing sub-cutaneous implants of MC38 colorectal carcinoma all responded to treatment with avelumab, while mice bearing either B16F10 melanoma or 4T1 breast cancer were both refractory to treatment.

A bilateral tumor model using CT26 was established, reasoning that immune phenotyping of one surgically resected tumor prior to avelumab treatment would provide information on immune parameters that correlate with response to treatment. Equal numbers of CT26 cells were implanted into both flanks of BALB/c mice and mice with similar sized tumors on each flank were enrolled onto observational study at 75 mm³ (±20 mm³), at which point further analysis was undertaken. Initially a growth curve characterization of bilaterally implanted mice was performed to determine the consistency in tumor volumes and response to avelumab treatment between tumors on the same mouse. A significant correlation in left and right tumor volumes for both isotype control- and avelumab-treated tumor pairs was observed, confirming the predictive potential of the bilateral model. This observed linearity also highlighted that there is more variability between mice than within mice irrespective of treatment vis-a-vis bilateral tumor volumes. Furthermore, in an independent bilateral growth curve assay it was found that tumor volume at the start of measurements (day 10) did not influence tumor growth rate. Together these findings imply that additional host factors are responsible for this underlying variability.

Reasoning that differences in underlying tumor immune microenvironments between mice influence tumor growth, single cell RNA-Sequencing (scRNA-Seq) of CD45⁺ cells from 12 pairs of bilateral tumors 10 days post-implantation was performed to assess the consistency in early tumor immune infiltration. In total 36 cell clusters were identified (including three CD45⁻ stromal cell clusters), many of which were significantly correlated between tumor flanks. Hierarchical clustering of the correlation matrix derived by comparing frequencies of these 36 tumor-infiltrating immune subsets showed that individual mice indeed clustered together, either as pairs or in small groups. Thus, there is a substantial influence of the host environment on tumor immune responses at distant sites. Tumor volume had much less influence on immune phenotype relative to individual mouse effects. Of the 36 cell subsets identified, two statistically significant associations exist with tumor volume. In contrast, 19 of 36 immune cell subsets were significantly correlated at the same p value cutoff (p<0.05) when comparing subset frequencies between left and right flanks. These data confirm that tumor immune profiles are consistent between flanks in bilaterally implanted mice, and that tumor growth rates are significantly affected by host-intrinsic effects.

To explore whether the variability in response of CT26 to avelumab was due to underlying differences in tumor immune microenvironments this bilateral tumor model was coupled with scRNA-Seq to permit profiling immune populations prior to treatment. Mice were implanted on both flanks, and tumors were grown to 78 mm³ (±19 mm³) before randomization (avelumab, n=26; isotype, n=10). Prior to treatment, one reference tumor on each mouse was surgically resected and processed for scRNA-Seq of fluorescence-activated cell sorting (FACS)-purified CD45⁺ cells. The growth rate of the second treatment tumor was then monitored for avelumab response. Responders were defined as mice with tumors that were smaller than the smallest tumor observed in the isotype control antibody-treated group at day 16, which corresponded to a size of 500 mm³.

scRNA-Seq data from all 30 avelumab-treated mice were analyzed together to define the pretreatment immune landscape. Unbiased clustering of variable genes was performed, resulting in a total of 26 CD45⁺ clusters, which were visualized using uniform manifold approximation and projection (UMAP). The same exact number of clusters as in the preliminary scRNA-Seq analysis of bilateral tumors sequenced at day was not observed. In both scRNA-Seq studies the tumors were sequenced at a consistent volume. Yet, a higher number of CD8 T cell clusters were observed in a preliminary analysis of bilateral tumor pairs, which were sampled day 10 post implantation, compared to the single-flank avelumab response study which was sampled at day 8. In the avelumab response study there were two large meta-clusters relating to the lymphoid and myeloid lineages as well as a distinct cluster composed of plasmacytoid dendritic cells (DCs). The identity of these 26 clusters was curated by manual review of known biomarkers and hierarchical clustering of differentially expressed genes, which was found to segregate cell clusters into groups consistent with their annotated identities. 10 clusters were identified, belonging to myeloid populations which were found to cluster together. These included TAMs and monocytes that express the genes Csf1r, Itgam (CD11b), and Adgre1 (F4/80) at differing intensities (M1-10). Five clusters were assigned a DC phenotype: two clusters of conventional DCs identified as Xcr1⁺Clec9a⁺Flt3⁺ (DC_1) and Xcr1⁻Clec9a⁻Flt3⁺ (DC_3) which share characteristics of DC clusters DC1 and DC3 defined by Zilionis et al (Zilionis et al., 2019), one myeloid DC cluster (DC_2) which was determined to be analogous to DC cluster DC2 in Zilionis et a/and two plasmacytoid DC clusters expressing Siglech and Csfr2b (DC_4 and 5). A further five clusters of T cells were identified. These include a mixed CD4/8-containing meta-cluster, T_1, that bears similarity to the group 1 tumor infiltrating T cell signature defined by Magen et al (Magen et al., 2019) which was described to express Cxcr6 and Ifng. CD4 T cell cluster T_3 expresses Tbx21, Cxcr3 and 117r, possessing a similar expression profile to the group 2 TILs defined by Magen et al, while cluster T_4 contains a proliferation signature alongside a mix of both T_1 and T_3 genes. Finally, two clusters of Foxp3 positive, Il2ra positive regulatory T cells (T_2 and 5) were observed. In addition, five clusters of natural killer cells (NK_1-5), and one B-cell cluster were also identified (B1). In order to understand which populations in the TME may respond to avelumab the expression of PD-L1 (Cd274) and PD-1 (Pdcd1) in scRNA-Seq clusters was examined. Low-level expression of PD-L1 was broadly observed, with more abundant expression on macrophage population M_3 and on the Xcr1⁻Clec9a⁻Flt3⁺ DC_3 cluster, whereas PD-1 expression was restricted to T cells, clusters (T_2, 3, 4 and 5).

### A TAM Subset Predicts Response to Avelumab Treatment

It was tested whether cell populations exist prior to treatment that can discriminate between responders and non-responders to avelumab-post surgery. Using UMAP, several clusters were observed with altered density distributions, reflecting potential changes in the frequencies of these populations. To capitalize on the variability in cell subset frequencies and responses, linear regression was performed by plotting the frequency of each cluster against the final tumor size after avelumab treatment. We did not observe any influence of starting tumor size in responder and non-responder populations, however, in 22 of 26 clusters a weak negative correlation exists between cluster abundance and tumor size, implying that immune infiltration before treatment corresponds to a higher rate of response. Of the 26 clusters, only two were significantly associated with tumor size, myeloid cluster M_3 and Treg cluster T_5.

Myeloid cluster M_3 was positively associated with response to treatment (R=−0.43; p=0.03); typically, mice with higher M_3 frequencies possessed smaller tumors. Stratifying mice into responder and non-responder populations reaffirmed this finding, showing that non-responders with the highest tumor volume following avelumab treatment possessed the fewest M_3 macrophages. Treg cluster T_5, however, was negatively associated with outcome (R=0.41; p=0.04), consistent with the role of Tregs in impairing antitumor immunity.

Notably, T cell clusters T_4 and T_5, myeloid cluster M_7 and NK cluster N_3 possessed a proliferation profile defined by mKi67 and Top2a expression. Thus, a proliferating Treg signature prior to anti-PD-L1 treatment is associated with worse outcomes. To understand if the frequency of proliferating Tregs could explain the presence of M_3, we investigated the correlation between these clusters and found that no such relationship exists (R=0.24; p=0.2; data not shown). However, the ratio of M_3 and T_5 was strongly predictive of tumor size (R=−0.71; p=<0.001).

It was examined whether genes expressed by individual cell clusters were associated with response to avelumab treatment. The analysis was focused on cytokine and TNF family members, reasoning that expression profiles of these critical immune regulators could provide insight into underlying molecular mechanisms associated with M_3 subset polarization. For each cluster gene expression profiles were identified that significantly correlated with tumor volume. 1110 was expressed by multiple TAM subsets (M_1, 2, 3, 5, 7, and 8) and was found to be positively correlated with final tumor size (i.e. associated with non-response to treatment), as was the expression of the Th2 cytokine 115 by T cell clusters T_1 and T_3. However, Ifng production by proliferating T cell cluster T_4, or Tnfsf9 (4-1 BB-L) by multiple lymphocyte subsets (T_1, 2, 4 or NK_2) was found to be associated with a more favorable response to avelumab. Thus, the immune landscape of tumor immune microenvironment predisposes to anti-PD-L1 responsiveness. Furthermore, a preexisting TAM population is associated with a favorable response to treatment. Differences in initial myeloid and Treg populations may coordinately regulate responses to immune checkpoint therapy.

### Cxcl9+ TAMs Predict Responsiveness to Avelumab Therapy

The positive correlation between myeloid M_3 and response to avelumab prompted investigation of the characteristics of this cluster that may underlie treatment response. First, myeloid and DC clusters were extracted, and visualization using t-distributed stochastic neighbor embedding (t-SNE) was performed, which provided a more even distribution of the clusters to aid phenotyping. The expression of monocyte, M1-like, and M2-like macrophage lineage markers was surveyed, including F4/80, CD11b, CD14, Ly6c, CD68, CD163, CD169, CD204, CD206, CD124, CCR2, CXCR3, and CXCR4, and Fcγ receptors, including FcγRIIb, FcγRIII, and FcγRIV. Most markers showed continuous expression spanning multiple myeloid populations that did not clearly discriminate between the individual cell populations defined by clustering. To better understand the phenotypic heterogeneity of tumor-associated macrophages and monocytes unbiased hierarchical clustering of variable genes (Table 5) and single cell trajectory analysis was performed. Each myeloid population possessed a distinct transcriptomic profile, however similarities in TAM cluster gene expression observed by hierarchical clustering were also supported by trajectory analysis, showing that these different TAM subsets show differing degrees of similarity. Cluster M_7 and M_9 share a proliferation-associated signature including expression of Mki67, Top2a and H2afx with M_7 expressing higher levels of these proliferation-associated genes (Table 5); clusters M_2 and M_4 possess a regulatory macrophage gene expression profile that includes Folr2, Maf, Retnla, Arg1, Mrc1 as well as complement factors C1qa and C1qb; cluster M_6 however also includes the Arg1 and Mrc1 gene expression cluster in addition to tissue remodeling genes including Mmp12 and Mmp13. Cluster M_5 were identified to be monocytes, uniquely expressing Vcan and Sell. M_5 shared a large cluster of genes with M_3 and M_8 TAMs, implying that these subsets may be recently monocyte-derived, these genes include Ly6C2, Nlrp3, Cxcl9 and Cxcl10 (Table 5). The single cell trajectory analysis also positions the M_3 and M_8 populations on the same branch as M_5 monocytes, further implying that these cells may have recent monocytic origins. Interestingly clusters M_3 and M_8 share an overlapping interferon-associated gene signature, that includes Cd40 and Ifi47, however, M_8 expresses additional interferon associated genes including Ifi441, Isg20, Ifit2, Ifit3 and Usp18 that are not observed in the M_3 population.

TABLE 5
Hierarchical Clustering of Variable Genes

| TAM Subset Cluster(s) | Exemplary Signature Gene(s) |
|---|---|
| M_7, M_9 | Mgl2, Cd81, Mki67, Top2a, H2afx |
| M_8 | Ifi44l, Isg20, Ifit2, Ifit3, Usp18 |
| Mono/M_5 | Vcan, Sell, Hp |
| M_6 | Ccl24, F10, Mmp12, Mmp13, Arg1, Mrc1 |
| M_2, M_4 | Abca1, Folr2, Maf, Retnla, Stab1, Arg1, |
| | Mrc1, C1qa, C1qb |
| Mono/M_5, M_3, M_8 | Cd40, Ifi47, Tor3a, Xaf1 |
| M_3 | Ly6l, Fam26f/Calhm6, Slamf8, Ccl5, M6pr, |
| | Cd38, Ly6a, Cd74, MHCII |
| Mono/M_5, M_3 | Cxcl9, Cxcl10, Ly6c2, Nlrp3, Ptgs2, Bhlhe40 |

To better understand how M_3 may control responses to avelumab, genes associated with M_3 compared with other tumor-associated macrophage clusters were identified. An activation profile was identified, with Cxcl9 being the most definitive gene for M_3. A broader inflammatory signature, including Cd40, Ccl5, M6pr, Cd38, Ly6a, Cd74, and MHCII gene expression, was also more abundant in M_3 than in other myeloid populations. Gene set enrichment analysis identified myeloid activation-associated pathways in M_3, showing enriched IFN-γ with relatively less interferon-α/β pathway utilization. This was supported by review of representative type-1 and type-2 interferon-associated genes Cxcl9 and Cxcl10 respectively, showing relatively higher expression of Cxcl9 in M_3 macrophages, yet higher Cxcl10 expression in M_8 TAMs. NF-κB and antigen presentation-associated gene were also upregulated in M_3 TAMs, however also observed in additional TAM subsets. Together these data strongly imply that IFNγ regulates M_3 polarization.

As M_3 macrophages correlate with response to avelumab treatment the frequencies of M_3 macrophages in other syngeneic models was assessed. We pooled and clustered scRNA-Seq data for MC38, 4T1 and CT26 and identified M_3 macrophages as the Cxcl9 and Cd40 expressing cluster 9. Consistent with the avelumab responsive nature of MC38 and CT26, higher frequencies of M_3 macrophages were observed relative to the refractory 4T1 tumors, furthermore the range of M_3 frequencies covering both responsive and non-responsive tumors in CT26 spans the range between 4T1 and MC38.

Strikingly, in gene lists recently published examining human TAM signatures by scRNA-Seq, one subset of TAMs, ‘M9’, shares notable similarity with TAM cluster M_3, containing, for example, Cxcl9, Cxcl10, Ccl5, MHC-II, Cd40 and Stat1 (Zilionis et al., 2019), seeming to provide a human ortholog to M_3. The similarity of these subsets was queried by performing enrichment analysis, comparing the overlap of gene lists defined for murine myeloid clusters M_1-M_10 to human TAM and monocyte subset gene lists defined in Zilionis et al.

Specifically, to examine correlates of M_3 and other macrophages in human datasets, gene expression data were downloaded from GSE127465 (Zilionis et al., 2019). Differentially expressed genes in each cluster were obtained and macrophage populations defined in the human dataset were compared with mouse macrophage subsets by mapping human and mouse gene identifiers using biomaRt. Similarities scores between gene lists for myeloid clusters in each dataset were compared by Fishers' exact test.

Many TAM subsets do not directly align in a one-to-one relationship, perhaps owing to species- and tissue-specific differences in macrophage polarization phenotypes as previously noted by Zilionis et al. As expected, however, substantial overlap was observed between classical monocyte gene sets in both our mouse and the Zilionis human datasets, consistent with the conserved one-to-one relationship between species (Zilionis et al 2019). Importantly, a similar highly significant intersection of gene lists is observed between the M_3 gene list we define here and the M9 signature defined by Zilionis et al, indicating that the M_3 TAM subset is conserved between species.

In summary, the inflammatory M_3 TAM population identified in this Example appears to be present in both mouse and in a related population in humans, and according to preclinical models, is predictive of the response to PD-1 axis antagonist treatment. A list of the genes whose expression is most highly correlated with M_3 and also possess conserved expression with this related human macrophage population is provided in Table 6.

TABLE 6
M_3 Myeloid Associated Genes

Cxcl9, Cxcl10, Fam26f/Calhm6, Gbp3,
Stat1, Plekho1, Gbp2, Lap3, PD-L1/Cd274,
Cd40, Tap1, Irf1, Tnfaip2, Psme2,
Atox1, Ube2l6, Scimp, Nfkbie, Psmb9, Tapbp,
Trafd1, Rnf19b, Pomp, Dram1, Slamf8,
Syngr2, Ccl5, Cd38, Nos2, M6pr, Tor3a,
Ptgs2, Socs3, Irf8, Cd273, Cd74, MHCII,
Ly6l, Xaf1, Nlrp3, Bhlhe40, Socs1, Cd86,

### Methods

Provided below are further details of methods of Example 1.

### Animals for In Vivo Studies

Six- to 8-week-old female Balb/c mice were purchased from Jackson Laboratories (Bar Harbor, Me.). All animals were housed in a pathogen-free vivarium facility at Pfizer Inc (South San Francisco and San Diego, Calif.), and experiments were conducted according to protocols in accordance with the Institutional Animal Care and Use Committee (IACUC) guidelines.

### Cells

The CT26 colon carcinoma cell line was purchased from American Type Culture Collection (ATCC CRL-2638; Manassas, Va.). Cells were cultured in Dulbecco's Modified Eagle Medium supplemented with 10% fetal bovine serum and 100 IU/mL penicillin-streptomycin at 37° C. in an atmosphere of 5% carbon dioxide and IMPACT tested for pathogens at the Research Animal Diagnostic Laboratory (Columbia, Mo.). Pathogen-free cells in exponential growth phase were harvested and used for tumor inoculation.

### Subcutaneous Tumor Models in Mice

Balb/c mice were inoculated subcutaneously on both flanks with an equal number of CT26 cells (1-5×10⁵) in 0.1 mL of phosphate-buffered saline (PBS). For the scRNA-Seq avelumab response study mice were randomized when tumors volume reached 78±19.1 mm³. Thirty animals were recruited to the avelumab treatment group and 10 to the isotype control antibody group. Treatment was started 24 hours after surgery. For the bilateral response to avelumab treatment study mice were treated day 10 post-injection (the same timepoint for the initial study), here tumors were 75 mm³±20 mm³. Tumor size was measured in two dimensions using a digital caliper, and the volume was expressed in cubic millimeters using the formula V=0.5×(L×W²) where L and W are the long and short diameters of the tumor, respectively. Body weight was recorded weekly. Percentage of tumor growth inhibition was defined as [1−(Tumor volume_{Treated}/Tumor volume_{Isotype control})]×100. Percentage of weight loss was defined as [1−(Body weight_{Treated}/Body weight_{Isotype control})]×100.

### Reagents

Avelumab was provided by Merck Serono (Darmstadt, Germany; Lot No. 508203). Human immunoglobulin G1 isotype control antibody was prepared in-house (Lot No. NB123249p192CA110124). Antibodies were prepared at a concentration of 1 mg/mL in PBS and dosed at 0.1 mL per mouse intraperitoneally for a total of three doses every 3. For CXCL9 and CXCR3 blocking studies antibodies were administered at the same dose and frequency as avelumab, however injections were performed one day prior to avelumab administration. For blocking antibody studies, anti-Cxcr3 clone Cxcr3-173 (Bioxcell) and anti-Cxcl9 clone MIG-2F5.5 (Bioxcell) were used.

### Single-Cell RNA-Seq and Gene Expression Quantification

Harvested tumors were dissociated to obtain single-cell suspensions using the mouse tumor dissociation kit (Miltenyi Biotec; Bergisch Gladbach, Germany) according to the manufacturer's protocol, except for Enzyme R, which was used at a 1:10 dilution of the protocol recommendation. For the scRNA-Seq avelumab response experiment, dissociated tumor cells were measured using Vi-CELL (Beckman Coulter; Brea, Calif.), and 1 to 5×10⁶ cells were stained using fluorescently labeled mouse CD45 antibody (clone 30F11) and Fixable Viability Dye eFluor 506 (Thermo Fisher Scientific; Waltham, Mass.) to sort for live CD45⁺ and CD45⁻ cells using the FACSAria II cell sorter (BD Biosciences; San Jose, Calif.). Approximately 5.5 to 111×10³ viable CD45⁺ cells were sorted with an 85-μm nozzle using default settings directly into round-bottomed polystyrene collection tubes containing 500 μL PBS with 0.5% bovine serum albumin (BSA). Sorted CD45⁺ cells were counted using the Cellometer K2 Viability Cell Counter (Nexcelom; Lawrence, Mass.) prior to loading on a Chromium Single Cell Chip (10x Genomics; Pleasanton, Calif.). For the bilateral paired scRNA-Seq study disassociated cells were filtered through 70 μm and 40 μm cell strainers and incubated with MACS Miltenyi CD45 TIL microbeads (Cat #130-110-618). Thereafter, CD45+ cells were isolated using the MACS Miltenyi multiMACS Cell Separator Plus (Cat #130-098-637), all according to manufacturer instructions. All cell counts were performed using the Beckman Coulter ViCell XR (Cat #731050). Following counts, cells were resuspended in 0.04% BSA in PBS at a concentration of 1e6 cells/ml. Approximately 16×10³ viable cells were loaded onto a Chromium Single Cell A Chip (10× Genomics) to aim for target cell recovery of 10⁴ cells. Library construction was performed using 50 ng cDNA following the Chromium Single Cell 5′ Library and Gel Bead Kit protocol (10×Genomics). Libraries were sequenced using the NovoSeq 6000 platform (Illumina; San Diego, Calif.). Data were processed using the Cell Ranger v2.1.1 (10× Genomics) to generate count-level data for further analysis. Each lane of cells was processed independently using the Cell Ranger count. The unique molecular identifier (UMI) counts for each sample were then merged using Seurat v2.3.3, requiring that the number of expressed genes for each cell was >200 and <5000. Cells with >10% of UMI originated from mitochondrial genes were removed. Genes expressed in at least three cells were kept and then normalized and scaled using the default setting in Seurat.

### Single-Cell Clustering and Annotation

For each sample, the top 2000 highly variable genes (HVG) were selected. These genes were combined into 3756 HVG for downstream analysis. Canonical correlation analysis (CCA) (Butler et al., 2018) was then performed to align cells belonging to the same cell type across different samples using the top 20 CCA components. Cell clustering was performed on the aligned CCA space. To find out the optimal cluster numbers, we repeated the clustering process using a sequence of resolution parameter from 0.2 to 1 in increments of 0.2, with larger values leading to a greater number of clusters. The optimal clustering was determined by the lowest increase in the cluster number when comparing each of two adjacent resolutions, which indicated a relatively stable result. This procedure generated 28 cell clusters at resolution 1.0. The cell identity was determined by manual review of top differentially expressed genes in each cell cluster. Two small CD45⁻ cell clusters indicating tumor or stromal cell contamination were removed from further analysis.

### Cell Cluster Frequency Calculation

For each sample, we calculated the frequency of cell cluster i in sample j as fᵢⱼ=nᵢⱼ/Σᵢ₌₁²⁶nᵢⱼ, where nᵢⱼ is the number of cells of cluster i in sample j calculated from our single cell sequencing data set, and Σᵢ₌₁²⁶nᵢⱼ is the total number of CD45⁺ cells sequenced in sample j. For simplicity, we named this frequency as % in45. Of note, it might be misleading when directly using this number in downstream analysis. First, because Σᵢ₌₁²⁶fᵢⱼ=1, it is not surprising to find a cluster's frequency going down, while another cell type's frequency going up. Second, different mice had different tumor sizes when measured even prior to treatment; however, single-cell sequencing protocols require a similar number of CD45⁺ cells collected for different samples. The value of % in45 without considering tumor size cannot faithfully reflect the profile of immune infiltration in tumors. To solve this issue, we instead calculated the percentage value of a cell cluster relative to the total live cell count prior to enrichment, named % inT, or % total live cells, in our downstream analysis. Specifically, % inT was defined as mᵢⱼ/Mⱼ, where mu is the number of cells of cluster i in tumor sample j, and M1 is the total number of all type of cells in tumor sample collected. M1 can be expressed as sum of CD45⁺ cells Mⱼ^{CD45+} and CD45⁻ cells Mⱼ^{CD45-}. Thus, % inT can be calculated as mᵢⱼ/Mⱼ=mᵢⱼ/Mⱼ^{CD45+} Mⱼ^{CD45+}/Mⱼ. In scRNA-Seq, a subset of Mⱼ^{CD45+} has been collected and sequenced, thus fᵢⱼ is an unbiased estimator of mᵢⱼ/Mⱼ^{CD45+}. The second term in the equation was derived from FACS when doing the cell sorting.

### Calculation of Correlation to Tumor Size

For each single-cell cluster, the correlation between % inT value of each cell cluster and tumor size were tested using linear regression. For all possible pairs of cell clusters, we calculated the log₂ ratio of the two % inT values, then linear regression was applied to test the association between the log₂ ratio and tumor size.

scRNA-Seq Assessment of Immune Subset Infiltration in Bilateral Tumor Pairs

Bilateral implantations, scRNA-Seq cell and library preparation and sequence data processing were performed as outlined above. Cell subset (cluster) frequencies defined based on automated cluster annotation calls were enumerated for within each tumor sample and linear regression analysis was performed to evaluate tumor infiltrating immune cell subsets between tumor pairs on the same mouse, or against tumor volume for each individual tumor. For hierarchical clustering analysis cell subset frequencies were standardized by subtracting the population mean from each cluster and dividing mean-centered values by row standard deviation to account for differences in cluster frequency abundance. A pairwise correlation matrix was calculated to compare infiltration profiles between individual tumors and heatmap visualization performed using complete linkage clustering hierarchical clustering or eucledian distance estimates.

### Myeloid Cell Heterogeneity Analysis

Cell clusters originating from myeloid lineage populations were extracted from the CD45⁺ cells for further analysis. t-SNE was applied to the myeloid lineage cells using Seurat. Due to the higher reproducibility and better meaningful organization of cell clusters (Becht et al., 2018), we chose the UMAP technique in our initial visualization of CD45⁺ cell clusters. The unsupervised single-cell trajectory for myeloid cell clusters was constructed in Monocle v2.6.4 (Trapnell et al., 2014), using the top 1000 differentially expressed genes. Gene Set Enrichment Analysis (GSEA) was applied to find pathway difference between C5 and other myeloid cell clusters. For better visualization in t-SNE, we defined a pathway score for each single cell using a similar method to (Kurtulus et al., 2019). First, the data were scaled (z-score across each gene) to remove bias toward highly expressed genes. Then, for each cell, a score was computed by first sorting the scaled expression value for each cell followed by summing up the indices of the pathway genes. For hierarchical clustering analysis differentially expressed genes were identified between all pairwise clusters, we selected genes that were differentially expressed between each TAM cluster and any other TAM cluster and further filtered genes whose expression was less than 2-fold greater than median TAM expression levels and an absolute expression level lower than 1 UMI. Row (gene) standardized expression levels were subject to hierarchical clustering for visualization.

### Example 2: Assessment of Expression Level of M_3 TAM-Associated Genes in Patient Samples and Patient Response to Immune Checkpoint Therapy

This Example describes the identification of components of the M_3 inflammatory gene signature that may contribute to the anti-tumor function of these cells and/or which are associated with patient response to immune checkpoint therapy.

To define components of the M_3 inflammatory gene signature that may contribute to the anti-tumor functions of these cells, clinical trial data sets for the PD-L1 antagonists avelumab and atezolizumab were interrogated. Univariate survival analysis was performed on expression of various M_3 genes in baseline tumors from two anti-PD-L1 clinical trials: IMvigor210, a phase II trial of atezolizumb in platinum-treated locally advanced or metastatic urothelial carcinoma, and EMR 100070-001, an EMD Serono phase I study of avelumab in solid tumors. For IMvigor210, there were baseline tumor samples from 348 patients, and clinical information and gene expression were obtained from the R package IMvigor210CoreBiologies. For EMR 100070-001, there were baseline tumor samples from 173 patients. In each data set, we divided the samples into three groups: high expression of the respective gene (greater than upper quantile), intermediate expression of the respective gene (less than upper quantile and greater than lower quantile), and low expression of the respective gene (less than lower quantile).

Patients were stratified into cohorts based upon expression of the M_3-associated genes Cxcl9, Cxcl10, Nos2, and Cd40 and assessed survival by Kaplan-Meier analysis. Strikingly, for avelumab and atezolizumab, respectively, we observed a 2.4- and 2.8-fold increase in median overall survival in patients with the highest Cxcl9 expression compared with those in the lowest-expressing quantile. We also observed a significant, albeit less profound, increase in survival times in patients with higher Cxcl10 expression in the atezolizumab trial; however, this trend was not observed for Nos2 or Cd40 expression levels. Cxcl9 and Cxcl10 are ligands for Cxcr3. Cxcr3 is required by T cells for effective antitumor immunity (Chow et al., 2019; Mikucki et al., 2015).

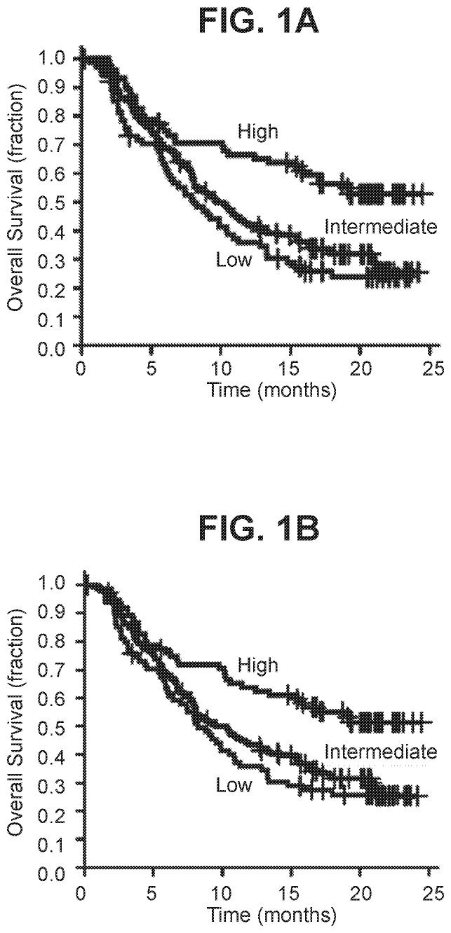

Next, univariate survival analysis was performed on expression of various M_3 genes in baseline tumors from the IMvigor210 urothelial carcinoma trial, using clinical information and gene expression were obtained from the R package IMvigor210CoreBiologies. Patients were stratified into high, intermediate, and low expression cohorts (as above) for combined expression of various groups of M_3-associated genes: Group A) Irf1, Stat1, and Gbp2, and Group B) Irf1, Stat1, Gbp2, and Tap1, and assessed survival by Kaplan-Meier analysis. To account for differences in the absolute expression of each gene, expression levels were median normalized before computing the average expression of each set (Group A and Group B respectively). In patients having high expression of Group A (FIG. 1A) and Group B (FIG. 1B), there was a 51% and 35% decreased risk of death, respectively, in patients with highest expression of the genes compared with those in the lowest expressing quantile. (Hazard ratios; Group A high quartile vs rest=0.494, Group B=0.653 high quartile vs rest=0.494. Risk of death calculated as 100*(1-HR)).

Given these clinical observations and our understanding that M_3 macrophages are the major source of Cxcl9 prior to treatment with anti PD-L1, we tested the role of Cxcl9 in mediating the response to avelumab. Mice were implanted on a single flank with CT26 tumors and pretreated with an anti-CXCL9 blocking antibody prior to avelumab treatment and monitored for tumor growth. Anti-CXCL9 treatment alone had no impact on tumor growth. However, CXCL9 blockade abolished tumor growth inhibition caused by avelumab. Consistently, inhibition of CXCL9 signaling by antagonizing the counterpart receptor CXCR3 yielded similar results.

Collectively, these findings demonstrate that a preexisting inflammatory macrophage signature predetermines responsiveness to anti-PD-L1 checkpoint therapy, that expression of Cxcl9 is a key determinant of responsiveness to immune checkpoint therapy such as PD-1 binding antagonists, and furthermore that expression of M_3 genes such as Cxcl9, Irf1, Stat1, and Gbp2 are strongly predictive of response to anti-PD-L1 treatment in clinical samples.

### Example 3: Metabolic Pathway Activity and Association with Treatment-Specific Effects on PFS

The randomized phase 3 JAVELIN Renal 101 trial (NCT02684006) demonstrated prolonged progression-free survival (PFS) with the combination of avelumab (anti-PD-L1)+axitinib (tyrosine kinase inhibitor [TKI] targeting vascular endothelial growth factor [VEGF] receptors [VEGFRs] 1, 2, and 3) vs sunitinib (multitarget TKI) in previously untreated patients with advanced renal cell carcinoma (aRCC). Details of the trial and results are provided in, for example, PCT/EP2020/065038, filed May 29, 2020, which is hereby incorporated by reference for all purposes.

A study was performed to explore the biology associated with response to treatment with combination immune checkpoint inhibitor (ICI)+VEGFR TKI or VEGFR TKI alone in aRCC, using data from the JAVELIN Renal 101 trial. The study prospectively defined molecular analyses of baseline tissue samples from patients enrolled in JAVELIN Renal 101 and their correlation with investigator-assessed PFS, based on a data cutoff of Jun. 20, 2018. Most tissue samples analyzed (≈63%) were collected during nephrectomy; the remainder were from metastatic sites. Through whole exome sequencing (WES), gene expression profiling, and immunohistochemistry (IHC), features were defined that differentiate outcomes for first-line ICI+VEGFR TKI combination therapy in aRCC, provide a novel basis for additional biological investigations into the responses to these agents, and inform personalized therapy strategies for patients with aRCC.

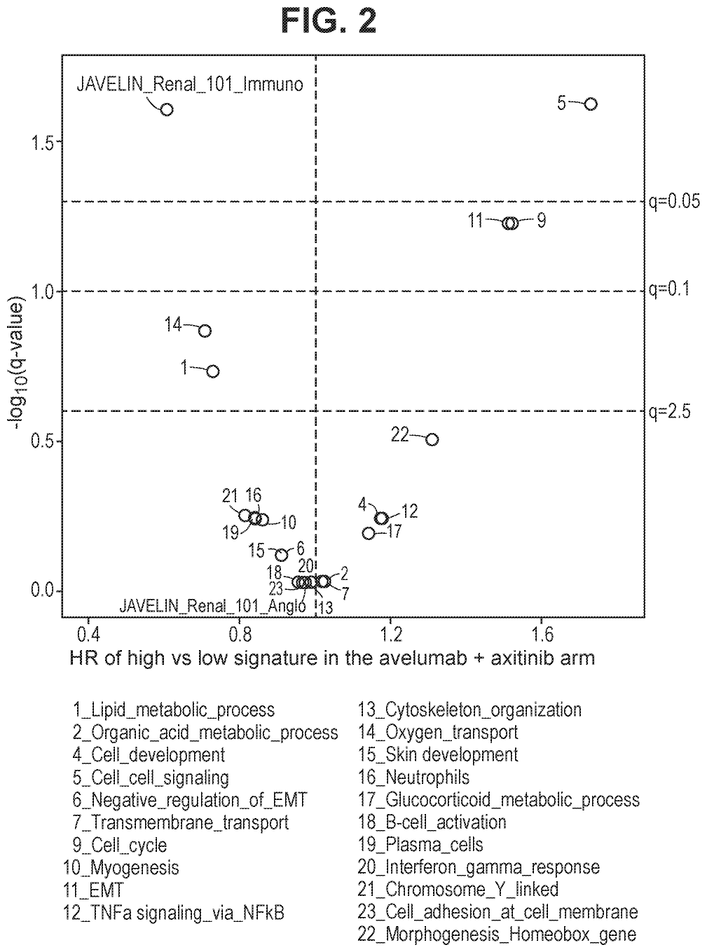

A co-expression network analysis identified biological network modules from baseline transcriptomic data. Network analyses identified 23 clusters, including 4 metabolic pathways: oxygen transport, lipid metabolism, organic acid metabolism, and glucocorticoid metabolism. Oxygen transport and lipid metabolism had a nominal p-value<0.05 in the survival model, and higher expression was associated with a trend toward improved PFS in the combination arm (FIG. 2).

Evaluation of KEGG metabolic pathways (i.e., glycolysis, TCA/Kreb cycle, fatty acid biosynthesis, pentose phosphate, and AMPK signaling pathways) previously associated with survival in RCC found no significant association with PFS in the combination arm. However, for the glycolysis and pentose phosphate pathways, the trend was consistent with the reference article. In the sunitinib arm, four pathways (TCA/Kreb cycle, fatty acid biosynthesis, pentose phosphate, and AMPK pathways) were consistent with the reference article, with the TCA cycle and AMPK pathways reaching significant nominal p-values.

Gene expression-based pathway analysis confirmed expected biology for the sunitinib arm, such as the significance of cell cycle and EMT pathways, but also identified positive associations between PFS and upregulation of oxygen transport and lipid metabolism pathways in the combination arm. As both are known to regulate and enhance innate immunity, this finding corroborates the RNAseq and IHC data suggesting contributions from the innate immune system as determinants of response.

### Example 4: Association Between Exploratory Biomarkers and Clinical Outcome in Avelumab First-Line (1 L) Maintenance+Best Supportive Care (BSC) Vs BSC Alone for Advanced Urothelial Carcinoma (UC)

The phase 3 JAVELIN Bladder 100 trial (Clinicaltrials.gov ID: NCT02603432) evaluated avelumab as maintenance therapy following response or stable disease with 1 L platinum-based chemotherapy in patients with advanced UC. Eligible patients with unresectable locally advanced or metastatic UC without disease progression after 4-6 cycles of gemcitabine with either cisplatin or carboplatin were randomized 1:1 to receive maintenance avelumab (10 mg/kg IV every 2 weeks)+best supportive care (BSC) or BSC alone, stratified by best response to 1 L chemotherapy (complete/partial response versus stable disease) and by visceral versus nonvisceral disease when initiating 1 L chemotherapy. The primary endpoint was OS, assessed from randomization in 2 primary populations: all randomized patients and patients with PD-L1+ tumors (Ventana SP263 assay). Secondary endpoints included PFS, objective response, and safety. 700 patients were randomly assigned to maintenance avelumab+BSC (n=350) or BSC alone (n=350) and were followed for a median of 19.6 and 19.2 months, respectively. Overall, 358 (51%) had PD-L1+ tumors. Avelumab+BSC significantly prolonged OS vs BSC alone in all randomized patients (hazard ratio [HR] 0.69; 95% CI 0.56, 0.86; 1-sided p=0.0005); median OS with avelumab+BSC vs BSC alone was 21.4 vs 14.3 months, respectively. Avelumab+BSC also significantly prolonged OS vs BSC alone in patients with PD-L1+ tumors (HR 0.56; 95% CI 0.40, 0.79; 1-sided p=0.0003); median OS was not reached vs 17.1 months, respectively. An OS benefit was also observed across all prespecified subgroups. JAVELIN Bladder 100 met its primary objective, demonstrating significantly prolonged OS with 1 L maintenance avelumab+BSC vs BSC alone in advanced UC in all randomized patients and patients with PD-L1+ tumors. Efficacy benefits were seen across all prespecified subgroups, and the safety profile of avelumab was consistent with previous studies of monotherapy. See also Powles T, et al. J Clin Oncol. 2020; 38 (suppl):Abstract LBA1.

The JAVELIN Bladder 100 trial was further analyzed to evaluate the predictive utility of several potential UC biomarkers. Tumor tissue was collected from primary or metastatic lesions prior to 1 L chemotherapy. Tumor tissue was subjected to biomarker analysis by either immunohistochemistry (IHC) or next-generation sequencing. Ad hoc exploratory analyses of associations between biomarkers and OS were conducted. Pre-specified thresholds were used to evaluate associations with PD-L1 expression by tumor cells or immune cells. Median biomarker values were used to set exploratory thresholds to reduce imbalance between subgroups. To evaluate PD-L1 expression, IHC was performed (Ventana SP263), where the threshold for PD-L1 positive was greater than or equal to 25% PD-L1 positive tumor cells or immune cells. To evaluate tumor mutuation burden (TMB), whole-exome sequencing was performed (Illumina NovaSeq). To evaluate gene alterations and polymorphisms, whole-exome sequencing was performed (Illumina NovaSeq). For gene expression profiling, whole transcriptome RNA-seq was performed (Illumina NovaSeq).

Cancers can be caused by the accumulation of somatic mutations; these can result in the expression of neoantigens. Tumor mutational burden (TMB) analysis can provide information regarding patient subgroups. In this analysis, TMB was determined using whole exome sequencing. A median of 7.66 mutations/Mb pairs was used to define subgroups. An overall survival benefit from avelumab was observed in the subgroup with a TMB above the median, even in the subgroup defined as PD-L1 negative by the SP263 test. Also, patients with a TMB below the median still showed an OS benefit if they were PD-L1 positive.

A summary of the hazard ratio (HR) in different patient subgroups relating to PD-L1 and/or TMB status is provided below in Table 7. In this analysis, the smaller the HR value for a subgroup, the greater the benefit observed for treatment of the group with avelumab+BSC as compared to BSC alone.

TABLE 7

| | HR (95% CL) Avelumab + BSC |
|---|---|
| Subgroup | vs. BSC alone |
| PD-L1 positive | 0.56 (0.400-0.790) |
| PD-L1 negative | 0.85 (0.616-1.181) |
| TMB high | 0.48 (0.332-0.707) |
| TMB low | 0.88 (0.643-1.197) |
| PD-L1 positive; TMB high | 0.51 (0.305-0.868) |
| PD-L1 positive; TMB low | 0.60 (0.382-0.955) |
| PD-L1 negative; TMB high | 0.44 (0.251-0.768) |
| PD-L1 negative; TMB low | 1.27 (0.799-2.006) |

In addition to TMB, the type and location of mutations may also play a critical role in determining outcomes. Tumors were scored according to the signatures of single base pair substitutions in the Catalog of Somatic Mutations in Cancer (COSMIC) database V2 (Wellcome Sanger Institute, UK; see, e.g. Tate, et al, Nucleic Acids Research, Vol 47., Issue D1, 8 Jan. 2019; pp D941-D947). In this analysis, it was determined that certain predefined signatures (e.g. Signature 23) were associated with lower HRs, while other predefined signatures (e.g. Signature 28) were associated with higher HRs, suggesting that different mutation processes may differ in their impact on tumor responsiveness to avelumab.

Exemplary COSMIC signatures, and the HR associated with greater than the median or less than or equal to the median mutation level of the respective signature in patients treated with avelumab+BSC are provided in Table 8.

TABLE 8

| Subgroup | HR (95% CL) Avelumab + BSC |
|---|---|
| Signature 23 > Median | 0.48 (0.335-0.699) |
| Signature 23 ≤ Median | 0.89 (0.645-1.226) |
| Signature 7 > Median | 0.58 (0.409-0.819) |
| Signature 7 ≤ Median | 0.79 (0.569-1.102) |
| Signature 2 > Median | 0.66 (0.464-0.934) |
| Signature 2 ≤ Median | 0.69 (0.500-0.960) |
| Signature 28 > Median | 0.98 (0.633-1.515) |
| Signature 28 ≤ Median | 0.57 (0.429-0.767) |

Elevated scores (i.e. >median) for signatures characterized by cytosine to thymine transitions (such as Signature 23), were associated with lower hazard ratios. In contrast, elevated scores for a signature characterized by thymine to guanine transversion (Signature 28) was associated with a higher hazard ratio. This suggests that different mutation processes may differ in their impact on tumor responsiveness to avelumab.

DNA damage response and repair (DDR) genes were also examined. Samples were analyzed for mutations in mismatch repair genes (MLH1, MSH2, MSH6, PMS1, PMS2), nucleotide excision repair genes (ERCC2, ERCC3, ERCC4, ERCC5), homologous recombination genes (BRCA1, MRE11A, NBN, RAD50, RAD51, RAD51B, RAD51 D, RAD52, RAD54L), Fanconi anemia genes (BRCA2, BRIP1, FANCA, RANCC, PALB2, RAD51C, BLM), checkpoint genes (ATM, ATR, CHEK1, CHEK2, MDC1), and other DNA damage response/repair genes (POLE, MUTYH, PARP1, RECQL4). This analysis found that mutations in DNA damage response and repair genes were associated with reduced hazard ratios, relative to wildtype. [HR with DDR mutated: 0.65 (0.504-0.847); HR with DDR wildtype: 0.89 (0.489-1.612)]. This is consistent with the hypothesis that mutations in critical genes may affect tumor responsiveness to avelumab (i.e. tumors having mutations in DDR genes may be more responsive to avelumab than tumors not having mutations in DDR genes).

Gene expression analysis of tumor cells was performed. The relationship between gene expression level and hazard ratio (HR) for the avelumab+BSC arm and the BSC arm was examined. The top 5 expressed genes in the avelumab+BSC arm associated with a HR below 1 are CD8, CXCL9, IFN-gamma, LAG3, and TIGIT. Other genes associated with a HR below 1 and an uncorrected p-value threshold of 0.001 or greater are: GBP4, GBP1, CXCL10, FOXP3, TBX21, PDCD1, NKG7, CXCR3, ITGAE, BATF2, HLA-DMA, CTLA4, CXCL11, IL2RB, GZMA, ICOS, CD274 (PD-L1), IL12RB1. The avelumab+BSC arm has many more genes that cross the uncorrected p-value threshold of 0.001 (i.e. greater than 0.001), as compared to the BSC arm (in which only the genes CCL28 and SNCAIP have a p-value greater than 0.001). This observation is consistent with the hypothesis that response to avelumab as a targeted agent is contingent on a spectrum of immune activated factors in the tumor in a way that best supportive care is not. In addition, most of the genes associated with lower HR in the avelumab+BSC arm were associated with immune responses. This is not the case for the BSC only arm, where none of these genes appeared.

Immune cell gene expression was also analyzed. The relationship between immune cell gene signatures as described in the leukocyte gene signature matrix (LM22) (Newman et al, Nat. Methods 2015; 12: 453-7) and overall survival (OS) in the avelumab+BSC arm was examined. Patients were assigned to above median and below median subgroups based on individual signature scores for different immune cell gene signatures. The different immune cell gene signatures and HR values are summarized below in Table 8.

TABLE 8

| Signature Class | Signature | >Median HR | ≤Median HR |
|---|---|---|---|
| T cell | Follicular helper T cell | 0.53 | 0.92 |
| | γδ T cell | 0.52 | 0.90 |
| | CD4+ T cell | 0.55 | 0.86 |
| | Regulatory T cell | 0.54 | 0.88 |
| | CD8+ T cell | 0.55 | 0.88 |
| | Memory activated | 0.56 | 0.84 |
| | CD4+ T cell | | |
| NK cell | Activated NK cell | 0.53 | 0.90 |
| | Resting NK cell | 0.54 | 0.90 |
| B cell | Naïve B cell | 0.57 | 0.89 |
| | Plasma cell | 0.64 | 0.78 |
| Macrophage | Macrophage M1 | 0.56 | 0.84 |
| Dendritic cell | Activated dendritic cell | 0.59 | 0.82 |

As shown in Table 8, elevated gene expression for multiple innate and adaptive immune cell types were associated with lower HR values. This suggests that multiple aspects of the immune response may contribute to the OS benefit from BSC+avelumab (as compared to BSC alone).

Some of the immune cell signatures correspond to immune cell types (NK cells, macrophages, and dendritic cells) that express the Fc-gamma receptors IIA (CD32A) and IIIA (CD16A). These Fc-gamma receptors are able to bind to the IgG1 Fc region of avelumab. Each of these receptors have variants that bind to IgG1 with higher or lower affinity. Accordingly, the association between the number of alleles encoding higher affinity Fc-gamma receptors and OS benefit from avelumab was examined.

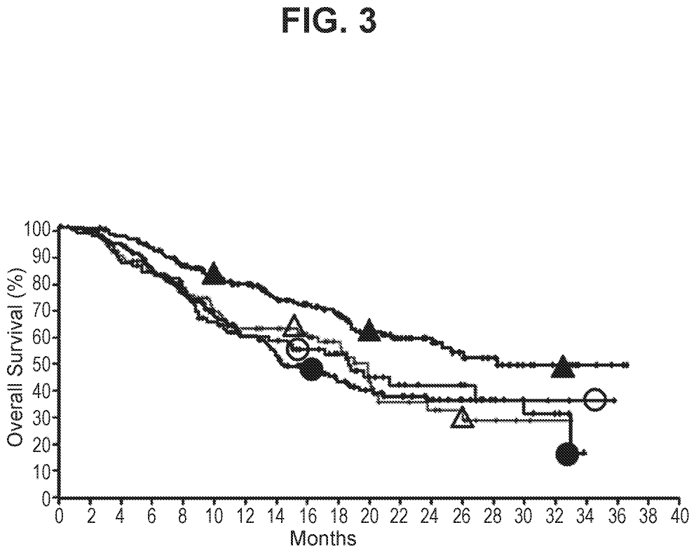

The number of loci encoding high-affinity FcgR variants FCGR2A and FCGR3A was determined from whole exome sequencing of the tumor. The number of high affinity alleles encoded at each locus was determined, and patients were assigned to subgroups based on the total number across both loci. FIG. 3 depicts the % OS over time of subjects having at least two high-affinity FcgR variant alleles or less than two high-affinity FcgR variant alleles, treated with either avelumab+BSC or BSC alone. The lines in the graph are labeled: solid triangle: avelumab+BSC and at least two high-affinity FcgR variant alleles; empty triangle: avelumab+BSC and less than two high-affinity FcgR variant alleles; solid circle: BSC alone and at least two high-affinity FcgR variant alleles; empty circle: BSC alone and less than two high-affinity FcgR variant alleles. As shown in FIG. 3, a significant OS benefit in the avelumab+BSC arm was observed in patients with at least two high-affinity alleles, which was not observed in patients in the avelumab+BSC with fewer than two high-affinity alleles. This observation is consistent with the hypothesis that antibody Fc region-immune cell FcgR interactions may contribute to OS benefit.

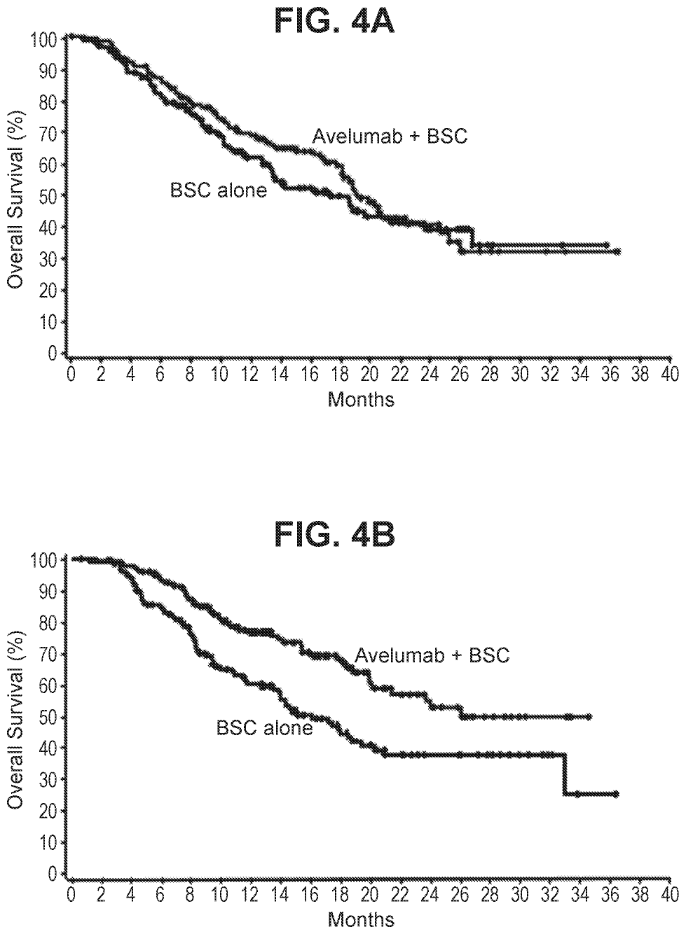

Next, the relationship between outcomes in the JAVELIN Bladder 100 trial and immune activity gene signatures was examined. The JAVELIN Immuno gene signature is a 26-gene signature, initially identified in the phase 3 JAVELIN Renal 101 trial described in Example 3 (see, Choueiri T K, et al. J Clin Oncol. 2019; 37:(suppl); Abstract 101; see also PCT/EP2020/065038, filed May 29, 2020). Using whole-transcriptome RNA-Seq data, JAVELIN Immuno scores were determined, and patients were assigned according to whether their scores were above or below the median. OS % over time for patients treated with avelumab+BSC or BSC alone and having JAVELIN Immuno scores less than or equal to the median are shown in FIG. 4A, and OS % over time for patients treated with avelumab+BSC or BSC alone and having JAVELIN Immuno scores greater than the median are shown in FIG. 4B. As shown in FIG. 4B, UC patients treated avelumab+BSC with scores above the median showed a significant OS benefit, suggesting that the JAVELIN Immuno signature may be broadly predictive across tumor types.

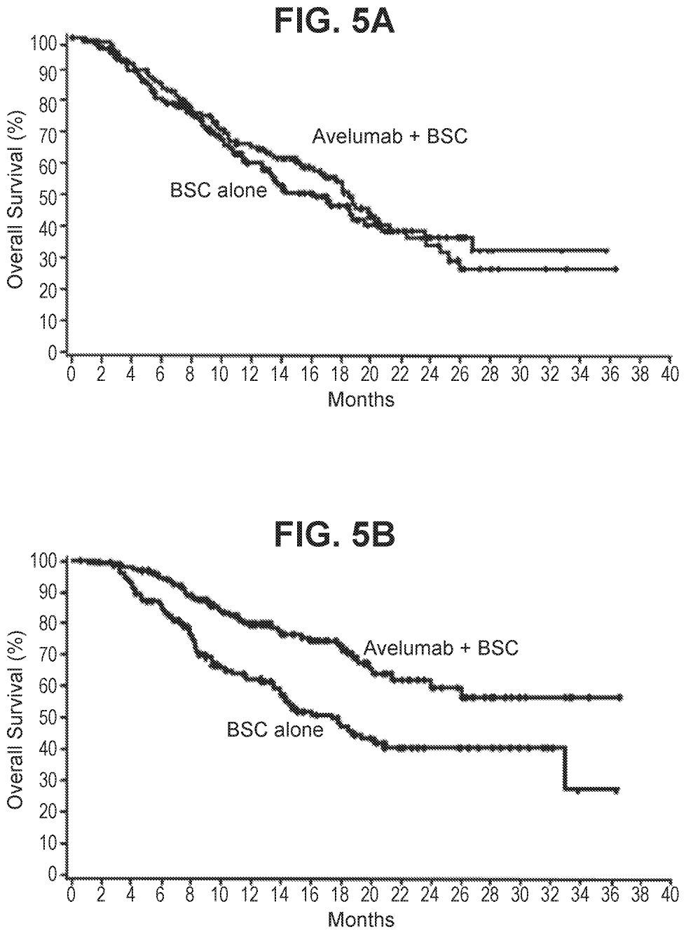

The relationship between outcome in the JAVELIN Bladder 100 trial and the T cell-inflamed 18-gene signature described in Ayers M, et al. J Clin Invest. 2017; 127:2930-40 was also examined. Using whole-transcriptome RNA-Seq data, T cell-inflamed scores were determined, and patients were assigned according to whether their scores were above or below the median. OS % over time for patients treated with avelumab+BSC or BSC alone and having T cell-inflamed scores less than or equal to the median are shown in FIG. 5A, and OS % over time for patients treated with avelumab+BSC or BSC alone and having T cell-inflamed scores greater than the median are shown in FIG. 5B. As shown in FIG. 5B, UC patients treated avelumab+BSC with scores above the median showed a significant OS benefit (FIG. 5B, HR=0.49; FIG. 5A HR=0.94).

Although JAVELIN Bladder patients that had high JAVELIN Immuno scores had improved response to avelumab+BSC as compared to patients with low JAVELIN Immuno scores (as shown in FIGS. 4A and 4B), a subset of patients having high JAVELIN Immuno scores still failed to respond. Among all patients, the HR for patients having high JAVELIN Immuno scores (FIG. 4B) was 0.55, and the HR for patients having low JAVELIN Immuno scores (FIG. 4A) was 0.87. To better understand the non-responder subpopulation among patients having high JAVELIN Immuno scores, various signaling pathways were examined in patients having these tumors (both avelumab+BSC and BSC alone arms were included in this analysis). Specifically, the notch, hedgehog, TGFb, and angiogenesis pathway signatures as described in MSigDB Hallmark Gene Set Collection. Liberzon A, et al. Cell Syst. 2015; 1:417-25 were examined.

The pathway signatures, and the HR associated with greater than the median or less than or equal to the median expression of respective signature in patients in the high JAVELIN immuno score subgroup are provided in Table 9.

TABLE 9

| Signature | >Median HR | ≤Median HR |
|---|---|---|
| Notch | 0.59 | 0.42 |
| Hedgehog | 0.59 | 0.43 |
| TGFβ | 0.63 | 0.44 |
| Angiogenesis | 0.70 | 0.30 |

As shown in Table 9, patients that have high expression of genes in the notch, hedgehog, TGFb, and angiogenesis pathways have reduced response to anti-tumor agents, even in subjects that have high expression of the JAVELIN-immuno signature. This data suggests that molecules in these pathways may represent therapeutic targets for combination therapies with immune checkpoint agents such as avelumab for the treatment of cancers such as urothelial carcinoma.

Analysis was also performed to identify mutated genes that were associated with increased OS or decreased OS in the avelumab+BSC arm of the JAVELIN Bladder 100 trial. The criteria for prioritizing these genes was as follows. For each gene, four models were constructed: (1) a Cox proportional hazards (Cox PH) model on the full cohort including an interaction term between gene mutational status and treatment arm (2) a Cox PH model on the full cohort including an interaction term between gene mutational status and treatment arm, adjusting for covariates age, sex, and tumor mutation burden (TMB) (3) a Cox PH model on the avelumab+BSC arm (4) a Cox PH model on the avelumab+BSC arm, adjusting for covariates age, sex, and TMB. Genes were filtered based on the following criteria: A) Mutated at 5% or more in the cohort; B) Interaction term P-value<0.05 in both model (1) and model (2), testing for treatment specific differences of the mutations on overall survival (OS); C) P-value<0.05 in both model (3) and model (4), testing for significant effects the mutants have on OS in the avelumab+BSC arm.

Applying criteria A, B, and C yielded 27 prioritized genes whose mutations are predictive of avelumab maintenance in bladder cancer. Of the genes, 18 have Hazard ratio (HR)<1 in both model (3) and model (4)—they contain mutations that have a beneficial effect on OS in the avelumab+BSC arm; 9 have HR>1 in both model (3) and model (4)— they contain mutations that associate with worse OS in the avelumab+BSC arm. The genes for which mutations in the genes are associated with improved OS in the avelumab+BSC arm are: FRYL, FER1L5, NBEA, SACS, CEP350, SOX17, SSPO, TENM4, KCP, ARAP1, NUP160, PTPRS, C6ORF132, KIAA1551, EPB41, MYH4, TRIM10, and TOP2B. The genes for which mutations in the genes are associated with reduced OS in the avelumab+BSC arm are: DNAH1, AGRN, NHSL1, EPHB4, EYS, GNAS, PLXNC1, AKAP1, and TBCD.

### Example 5: Innate and Adaptive Immune Biomarkers Correlate with Better Survival with Maintenance Avelumab in Advanced Urothelial Carcinoma

Additional biomarker analysis was performed on specimens from patients from the JAVELIN Bladder 100 trial (described in Example 4) who received dose of study drug on the avelumab arm or completed cycle 1 day 1 on the BSC arm and had biomarker assessment performed on a tumor biopsy collected prior to chemotherapy.

Associations with Tumor Mutational Burden (TMB)

Potential immunogenicity associated with genomic alterations was evaluated using tumor whole-exome sequencing (WES) data. TMB, which is positively correlated with neoantigen content, was defined as the number of nonsynonymous single-nucleotide variants (SNVs)/megabase (Mb). The median value (7.66 nonsynonymous SNVs/Mb) was used as the threshold to define TMB-high subpopulations. HR for risk of death was lower in the TMB>median subgroup (HR=0.48; 95% CI (0.33, 0.71); P=0.0002; n=302) than in the TMB≤median subgroup (HR=0.88; 95% CI (0.64, 1.20); P=0.41; n=305), but the treatment-by-biomarker interaction P value was 0.26. The combination of TMB status based on median split with PD-L1 high/low status displayed a treatment-by-biomarker interaction P value of 0.01. In the PD-L1-high patient population, the HR for risk of death appeared to be similar in the TMB>median subgroup (HR=0.51; 95% CI (0.31, 0.87); P=0.01; n=190) compared with the TMB≤median subgroup (HR=0.60; 95% CI (0.38, 0.96); P=0.03; n=148). In contrast, in the PD-L1-low patient population, the HR for risk of death appeared lower in the TMB>median subgroup (HR=0.44; 95% CI (0.25, 0.77); P=0.004; n=105) than in the TMB≤median subgroup (HR=1.27; 95% CI (0.80, 2.01); P=0.32; n=140). These findings are consistent with the hypothesis that OS benefit from avelumab/BSC may be positively associated with elevated TMB, as has been noted in trials of other ICIs in UC, even when PD-L1 expression in the tumor microenvironment (TME) is low. Complex interactions between TMB and PD-L1 expression on TCs vs ICs are likely to complicate optimization and validation of use of these individual biomarkers for predictive testing, however, suggesting that additional biomarkers may be needed for optimal clinical use.

Associations with Mutational Signatures

De novo single-base substitution (SBS96) signatures were derived and decomposed to the Catalog of Somatic Mutations in Cancer (COSMIC) version 3.1 using the COSMIC Sig Profiler bioinformatics tools. All evaluable tumors in the specimen set contained SBS1 and SBS5, associated with spontaneous or clocklike mutation processes, respectively. A subset of tumors also contained SBS2 and/or SBS13, both of which are associated with mutagenic APOBEC (“apolipoprotein B mRNA editing enzyme, catalytic polypeptide-like”) activity. SBS2 and SBS13 tended to co-occur and were associated with elevated TMB. Comparison of tumors with either SBS2 and/or SBS13 signatures to tumors without either one identified APOBEC3B as the most differentially expressed gene between the groups, consistent with the hypothesis that APOBEC activity was contributing to mutagenesis. The association between mutation signature score and HR for risk of death (avelumab/BSC vs BSC) was most prominent for SBS2 (treatment-by-biomarker interaction term P value of 0.023 [SBS2 only] or 0.068 [SBS2 and SBS 13]) relative to SBS1, SBS5, or SBS7b (treatment-by-biomarker interaction term P value range, 0.568-0.858). An APOBEC mutation signature may have predictive utility, extending previous observations that APOBEC activity was positively associated with outcome in UC.

Associations with Mutations in Specific Pathways

Mutations in DNA damage response (DDR) genes have been associated with improved outcomes from ICI treatment in UC. Although mutations in many of these genes may have been positively associated with OS benefit from avelumab/BSC to varying degrees, individual mutations, including BRCA1 and BRCA2, either were too rare to assess or did not show P<0.05 for treatment-by-biomarker interaction. DDR mutations are associated with improved outcomes from platinum-based chemotherapy as well as ICI therapy, so it is possible that the effects of DDR mutations on OS benefit from maintenance avelumab are relatively weak compared with the effects on outcomes of platinum-based chemotherapy, as well as ICI therapy in other settings. Mutations in members of the fibroblast growth factor/fibroblast growth factor receptor (FGF/FGFR) family have been associated with reduced tumor infiltration by ICs and poor outcome from ICI therapy in UC; FGFR1 and FGFR3 and were mutated in 4.6% (28/607) and 19.4% (118/607) of evaluable patients, respectively. The treatment-by-biomarker interaction term P value was 0.75 for FGFR1 (HR for risk of death [mutant vs wild-type] in avelumab/BSC=0.92; 95% CI [0.34, 2.50]; P=0.87; n=310; vs HR for risk of death [mutant vs wild-type] in BSC=0.75; 95% CI (0.35, 1.60); P=0.45; n=297). The treatment-by-biomarker interaction term P value was 0.08 for FGFR3 (HR for risk of death [mutant vs wild-type] in avelumab/BSC=1.76; 95% CI [1.16, 2.66]; P=0.008; n=310; vs HR for risk of death [mutant vs wild-type] in BSC=1.03; 95% CI [0.69, 1.52]; P=0.90; n=297). The remaining FGF/FGFR family members, including FGFR2, were only present in ≤4.0% of the study population, and associations with OS were not estimated due to the limited number of events in subgroups with the mutations. The variable effect size associated with mutations in FGF/FGFR family members precludes specific recommendations regarding appropriate treatment of patients with these mutations. Additional searches for mutations based on prevalence (>5% of patients) and statistical significance criteria for association with OS benefit in the avelumab/BSC arm (P<0.05 in avelumab/BSC arm and interaction term P<0.05, with or without adjustment for age, sex, TMB, best response to first-line chemotherapy, and metastatic site when initiating first-line chemotherapy) identified genes representing multiple biological pathways, including DNA replication (e.g., TOP2A and LIG4), but did not define distinct phenotypes.

Taken together, the effect of genomic alterations on OS benefit from maintenance avelumab appears to be primarily related to increases in TMB with a distinct contribution from APOBEC activity. Assessment of the effect of mutations in specific genes, such as FGFR3 mutations in appropriately designed prospective studies, may be performed to understand implications for treatment of patients with these mutations.

Associations with Gene Expression

Relationships between individual gene expression as assessed by whole-transcriptome sequencing and HR for risk of death in the avelumab/BSC and BSC arms was examined. Many genes associated with immune responses, including CD8A, CD8B, IFNG, and CXCR3 and its ligands CXCL9 and CXCL10, were associated with OS benefit in the avelumab/BSC arm (HRs<0.9 per single-unit increase of gene expression; false discovery rate [FDR] q values<0.1) but not in the BSC arm (the treatment-by-biomarker interaction term P<0.05), consistent with other studies of ICIs in UC. TIGIT (treatment-by-biomarker interaction term P=0.0245) was of particular interest as a key component of an immune checkpoint network not frequently linked to outcome with ICIs in UC, and TIGIT is being targeted by agents in ongoing clinical studies (e.g., tiragolumab, OMP-313M32, BGB-A1217, M6223, domvanalimab, MK-7684 [TIGIT], COM701 [PVRIG]). Associations between OS benefit with avelumab/BSC and other members of the TIGIT network were therefore investigated. Of these members, PVRL1 expression displayed the lowest treatment-by-biomarker interaction term (P=0.0387). PVRL1 has been reported to enhance TIGIT signaling by stabilizing the TIGIT ligand PVR on the cell surface. The positive association of TIGIT with OS benefit may therefore reflect the presence of activated lymphocytes in the TME; reduction of the effect in the presence of TIGIT ligands suggest that inhibitors of the TIGIT pathway may provide further benefit in this setting.

Immune-related mechanisms that may be positively associated with OS benefit were further investigated using previously published gene sets representing lymphoid and myeloid subsets. Lower HR for risk of death was positively associated with several immune-related gene sets representing innate immunity (natural killer [NK] cells, macrophages, and dendritic cells) as well as adaptive immunity (B cells, CD4 T cells, and CD8 T cells). Four chemokine genes that were individually associated with OS benefit from avelumab/BSC with a treatment-by-biomarker interaction term P<0.05 (CCL8, CXCL9, CXCL10, and CXCL11) are members of a 12-chemokine gene signature associated with lymphoid structures in melanoma, suggesting that chemokine-mediated recruitment of multiple IC types to the TME could support the activity of avelumab. A gene signature characteristic of a CXCL9-producing macrophage subset described in Examples 1-2 herein displayed a notable positive association with OS benefit from avelumab/BSC (HR per 1-unit increase in signature score=0.29; 95% CI [0.16, 0.54]; P<0.001; n=282) relative to BSC (HR per 1-unit increase in signature score=0.92; 95% CI [0.54, 1.58]; P=0.77; n=275), with a treatment-by-biomarker interaction term P value of 0.006.

Five immune gene signatures validated in previous clinical studies of IC's or radical cystectomy and adjuvant chemotherapy were associated with a lower HR for risk of death in the avelumab/BSC arm relative to the BSC arm (treatment-by-biomarker interaction P-value range, 0.0061-0.0803). No single gene was shared by all five signatures, although CXCL9 was present in four out of five. These findings, taken together, suggest that the OS benefit from avelumab may be associated with a network of innate as well as adaptive immune mechanisms in the TME, with CXCL9 possibly playing a key role.

A similar approach was taken to characterize broader biological mechanisms that may have an impact on OS benefit, using the Molecular Signatures Database hallmark gene set collection. Hallmark sets representing the interferon-γ response and the interferon-α response displayed the lowest treatment-by-biomarker interaction term P values (0.0417 and 0.0361, respectively), as expected from the immune gene signature analysis. None of the other gene sets presented treatment-by-biomarker interaction term P values<0.05. Reduced ICI benefit in UC has been previously associated with multiple pathways connected to tissue growth, such as transforming growth factor β (TGF-β) and interleukin-8, in addition to gene signatures of myeloid cells and fibroblasts. Inhibitors of the TGF-β pathway (e.g., vactosertib and bintrafusp alfa) and angiogenesis pathways (e.g., axitinib, cabozantinib, lenvatinib, and bevacizumab) are currently being evaluated in UC. To investigate whether these or other pathways might be associated with reduced OS benefit from avelumab in the presence of an immunologically active TME, the hallmark analysis was repeated in the patient subgroup with greater-than-median values of the JAVELIN-Immuno signature. The evaluated hallmark gene sets did not display treatment-by-biomarker interaction term P values<0.09: however, some pathways, such as Notch signaling, angiogenesis, and TGF-β, may have negative associations with OS benefit from avelumab. It is possible that such interventions in these pathways may improve OS benefit from maintenance avelumab following first-line chemotherapy, but additional studies are needed to confirm the association and quantify the effect size for the purpose of prioritizing possible combination therapies.

### Development of Multiparameter Models Based on Mutation and Gene Expression

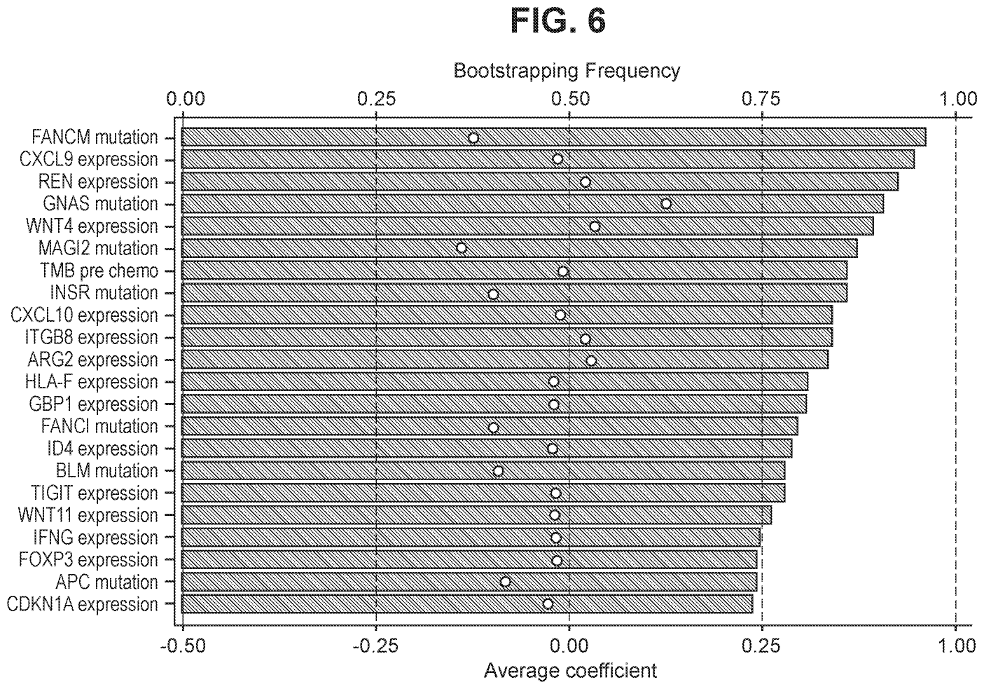

These analyses indicate that OS benefit from maintenance avelumab is associated with many molecular biomarkers representing multiple biological pathways. To identify a smaller group of biomarkers that may be most informative for research and clinical use, multiparameter models were developed to maximize retention of correlated biomarkers (e.g., biomarkers representing multiple arms of the immune response) while reducing variance and overfitting due to inclusion of excess features. Elastic net (EN) modeling is a well-established, regularized regression method that optimizes both the feature-retention properties of ridge and the feature-elimination properties of lasso. EN modeling was applied to a subset of gene mutation and gene expression data that may be obtained from commercially available targeted platforms. The resulting 22-feature model included TMB, mutations in seven genes, and expression of 14 genes (FIG. 6) that, taken together, represent genomic alterations, immune response, and tissue growth pathways (Table 10).

TABLE 10
22-feature model components

| # | Feature | Class |
|---|---|---|
| 1 | TMB (tumor mutational burden) before | Cancer genome |
| | chemotherapy | alteration |
| 2 | FANCM (Fanconi anemia complementation | Cancer genome |
| | group M) mutation | alteration |
| 3 | FANCI (Fanconi anemia complementation | Cancer genome |
| | group I) mutation | alteration |
| 4 | BLM (Bloom syndrome RecQ-like helicase) | Cancer genome |
| | mutation | alteration |
| 5 | Increased CXCL9 (C-X-C motif chemokine | Immunity: innate |
| | ligand 9) expression | and adaptive |
| 6 | Increased IFNG (interferon gamma) expression | Immunity: innate |
| | | and adaptive |
| 7 | Increased CXCL10 (C-X-C motif chemokine | Immunity: innate |
| | ligand 10) expression | and adaptive |
| 8 | Increased FOXP3 (Forkhead box protein P3) | Immunity: innate |
| | expression | and adaptive |
| 9 | Increased TIGIT (T cell immunoreceptor | Immunity: innate |
| | with Ig and ITIM domains) expression | and adaptive |
| 10 | Decreased ARG2 (arginase 2) expression | Immunity: innate |
| | | and adaptive |
| 11 | Wild-type GNAS (G protein subunit alpha S) | Immunity: innate |
| | | and adaptive |
| 12 | Increased GBP1 (guanylate binding protein 1) | Immunity: innate |
| | expression | and adaptive |
| 13 | Increased HLA-F (major histocompatibility | Immunity: innate |
| | complex, class I, F) expression | and adaptive |
| 14 | Decreased REN (renin) expression | Tissue growth |
| 15 | Increased WNT11 (wingless-type MMTV | Tissue growth |
| | integration site family, member 11) expression | |
| 16 | Increased ID4 (inhibitor of DNA binding 4, | Tissue growth |
| | HLH protein) expression | |
| 17 | Decreased WNT4 (wingless-type MMTV | Tissue growth |
| | integration site family, member 4) expression | |
| 18 | INSR (insulin receptor) mutation | Tissue growth |
| 19 | APC (adenomatous polyposis coli) mutation | Tissue growth |
| 20 | MAGI2 (membrane-associated guanylate kinase, | Tissue growth |
| | WW and PDZ domain containing 2) mutation | |
| 21 | Decreased ITGB8 (integrin subunit beta 8) | Tissue growth |
| | expression | |
| 22 | Increased CDKN1A (cyclin-dependent kinase | Tissue growth |
| | inhibitor 1A) expression | |

This model could identify patients with increased OS in two independent cohorts: (1) a holdout set from the avelumab/BSC arm (HR [higher than vs equal to or lower than median]=2.18; 95% CI (1.134, 4.190); P=0.0194; n=112) and (2) the IMvigor210 study (HR [higher than vs equal to or lower than median]=2.24; 95% CI (1.334, 3.749); P=0.0023; n=106). The 22-feature model did not identify patients with increased OS in the BSC arm (HR [higher than vs equal to or lower than median]=1.10; 95% CI (0.787, 1.540); P=0.5734; n=274), suggesting that this model was predictive rather than prognostic.

All references cited herein, including patents, patent applications, papers, text books, and the like, and the references cited therein, to the extent that they are not already, are hereby incorporated by reference in their entirety. In the event that one or more of the incorporated literature and similar materials differs from or contradicts this application, including but not limited to defined terms, term usage, described techniques, or the like, this application controls.

The foregoing description and Examples detail certain specific embodiments of the invention and describes the best mode contemplated by the inventors. It will be appreciated, however, that no matter how detailed the foregoing may appear in text, the invention may be practiced in many ways and the invention should be construed in accordance with the appended claims and any equivalents thereof.

1. A method of treating a patient having a cancer, comprising administering to the patient a therapeutically effective amount of a PD-1 axis binding antagonist, wherein the expression level of at least one gene selected from the group consisting of Irf1, Stat1, and Gbp2 in a sample obtained from the patient has been determined to be increased as compared to a reference level prior to treatment.

2. The method of claim 1, wherein the expression level of at least two or all three of the genes selected from the group consisting of Irf1, Stat1, and Gbp2 in the sample obtained from the patient has been determined to be increased as compared to a reference level.

3. The method of claim 1, further wherein the expression level of at least one gene selected from the group consisting of Tap1, Psmb9, Ccl5 and Cd38 in the sample obtained from the patient has been determined to be increased as compared to a reference level.

4. The method of claim 3, wherein the expression level of at least two genes selected from the group consisting of Tap1, Psmb9, Ccl5 and Cd38 in the sample obtained from the patient has been determined to be increased as compared to a reference level.

5. The method of claim 1, further wherein the expression level of at least one gene selected from the group consisting of Slamf8, Calhm6, Cd40, Cxcl9, PD-L1, Nos2, M6pr, Cd74, MHCII, and Ly6I in the sample obtained from the patient has been determined to be increased as compared to a reference level.

6. The method of claim 1, further wherein the expression level of at least one gene selected from the group consisting of Cxcl10, Tor3a, Xaf1, Nlrp3, Ptgs2, Bhlhe40, Socs3, Socs1, Irf8, Cd86, Cd273, Gbp3, Plekho1, Lap3, Tnfaip2, Psme2, Atox1, Ube2I6, Scimp, Nfkbie, Tapbp, Trafd1, Rnf19b, Pomp, Dram1, and Syngr2 in the sample obtained from the patient has been determined to be increased as compared to a reference level.

7. A method of treating a patient having a cancer, comprising administering to the patient a therapeutically effective amount of a PD-1 axis binding antagonist, wherein the expression level of at least one gene selected from the group consisting of Slamf8 and Calhm6 in a sample obtained from the patient has been determined to be increased as compared to a reference level prior to treatment.

8. The method of claim 7, further wherein the expression level of at least one gene selected from the group consisting of Cd40, Cxcl9, and PD-L1 in the sample obtained from the patient has been determined to be increased as compared to a reference level.

9. The method of claim 1, wherein the sample comprises leukocytes.

10. The method of claim 9, wherein the sample comprises at least one cell type selected from the group consisting of CD45+ cells, tumor-associated myeloid cells, and tumor-associated macrophages.

11. (canceled)

12. A method of treating a patient having a cancer, comprising administering to the patient a therapeutically effective amount of a PD-1 axis binding antagonist, wherein a sample from the patient is pre-determined to have at least one of and optionally two or all three of the following characteristics:
(i) it has one or both of the following characteristics: a) a protein altering mutation in at least one, two, or all three genes selected from the group consisting of Fanci, Fancm, and Blm; b) an increased tumor mutational burden (TMB) as compared to a reference level;
(ii) it has at least one, two, or all three of the following characteristics: a) an increased expression level of at least 1, 2, 3, 4, 5, 6 or all 7 genes selected from the group consisting of Cxcl9, Ifng, Cxcl10, FoxP3, Tigit, Gbp1, Hla-F as compared to a reference level; b) a decreased expression level of the gene Arg2 as compared to a reference level; c) a wild-type version of the gene Gnas;
(iii) it has at least one, two, or all three of the following characteristics: a) an increased expression level of at least one, two, or all three genes selected from the group consisting of Wnt11, Id4, CDKN1A as compared to a reference level; b) a decreased expression level of at least one, two, or all three genes selected from the group consisting of Ren, Wnt4, and Itgb8 as compared to a reference level; c) a protein altering mutation in at least one, two, or all three genes selected from the group consisting of Insr, Apc, and Magi2.

13. (canceled)

14. The method of claim 1, wherein the respective reference level of gene expression or TMB is determined based on an average level of the gene expression or TMB from a plurality of samples from patients having the cancer.

15. The method of claim 1, wherein the respective reference level of gene expression or TMB is determined based on an average level of the gene expression or TMB from a plurality of samples from human subjects.

16. The method of claim 1, wherein the sample obtained from the patient is a tissue sample, a whole blood sample, a plasma sample, or a serum sample.

17. The method of claim 1, wherein the sample comprises or consists of tumor associated leukocytes, tumor associated CD45+ cells, tumor associated myeloid cells, or tumor associated macrophages.

18. The method of claim 1, wherein the PD-1 axis binding antagonist is an anti-PD-L1 antibody.

19. The method of claim 1, wherein the anti-PD-L1 antibody is selected from the group consisting of avelumab, atezolizumab and durvalumab.

20. The method of claim 1, wherein the cancer is bladder cancer, breast cancer, clear cell kidney cancer, lung squamous cell carcinoma, malignant melanoma, non-small-cell lung cancer (NSCLC), ovarian cancer, pancreatic cancer, prostate cancer, renal cell carcinoma, small-cell lung cancer (SCLC), triple negative breast cancer, acute lymphoblastic leukemia (ALL), acute myeloid leukemia (AML), chronic lymphocytic leukemia (CLL), chronic myeloid leukemia (CML), diffuse large B-cell lymphoma (DLBCL), follicular lymphoma, Hodgkin's lymphoma (HL), liver cancer, mantle cell lymphoma (MCL), multiple myeloma (MM), myelodysplastic syndrome (MDS), non-Hodgkin's lymphoma (NHL), Squamous Cell Carcinoma of the Head and Neck (SCCHN), small lymphocytic lymphoma (SLL), endometrial cancer, B-cell acute lymphoblastic leukemia, colorectal cancer, glioblastoma, cervical cancer, penile cancer, or non-melanoma skin cancer.


SEQUENCE LISTING (24 sequences)

SEQ ID NO: 1 - protein, 444 aa, Artificial Sequence
  Synthetic Construct
QVQLVQSGAEVKKPGASVKVSCKASGYTFTSYWINWVRQAPGQGLEWMGNIYPGSSLTNYNEKFKNRVTMTRDTSTSTVYMELSSLRSEDTAVYYCARLSTGTFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 2 - protein, 443 aa, Artificial Sequence
  Synthetic Construct
QVQLVQSGAEVKKPGASVKVSCKASGYTFTSYWINWVRQAPGQGLEWMGNIYPGSSLTNYNEKFKNRVTMTRDTSTSTVYMELSSLRSEDTAVYYCARLSTGTFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 3 - protein, 221 aa, Artificial Sequence
  Synthetic Construct
DIVMTQSPDSLAVSLGERATINCKSSQSLWDSGNQKNFLTWYQQKPGQPPKLLIYWTSYRESGVPDRFSGSGSGTDFTLTISSLQAEDVAVYYCQNDYFYPHTFGGGTKVEIKRGTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 113 aa, Artificial Sequence
  Synthetic Construct
DIVMTQSPDSLAVSLGERATINCKSSQSLWDSGNQKNFLTWYQQKPGQPPKLLIYWTSYRESGVPDRFSGSGSGTDFTLTISSLQAEDVAVYYCQNDYFYPHTFGGGTKVEIK

SEQ ID NO: 5 - protein, 117 aa, Artificial Sequence
  Synthetic Construct
QVQLVQSGAEVKKPGASVKVSCKASGYTFTSYWINWVRQAPGQGLEWMGNIWPGSSLTNYNEKFKNRVTMTRDTSTSTVYMELSSLRSEDTAVYYCARLLTGTFAYWGQGTLVTVSS

SEQ ID NO: 6 - protein, 113 aa, Artificial Sequence; identical to SEQ ID NO: 4
  Synthetic Construct

SEQ ID NO: 7 - protein, 432 aa, Homo sapiens
QVQLVESGGGWQPGRSLRLDCKASGITFSNSGMHWVRQAPGKGLEWVAVRWYDGSKRYYADSVKGRFTISRDNSKNTLFLQMNSLRAEDTAVYYCATNDDYWGQGTLVTVSSASTKGPSVFPLAPCSRSTSESTAALGCLVDYFPEPVTVSWNSGALTSGVHTFPAVLQSSGLYSLSSVVTVPSSSLGTTYTCNVDHKPSNTKVDRVESYGPPCPPCPAPEFLGGPSVFLFPPKPKDTLMISRTPEVTCWVDVSQEDPEVQFNWYYDGVEVHNATKPREEQFNSTYRVVSVLTVLHQDWLNGKEYKCKVSNKGLPSSIEKTISKAGQPREPQVYTLPPSQEEMTKNQVSLTCLVKGFYPSDIAVEWESNGQPEKNYKTTPPVLDSDGSFFLYSRLTVDKSRWQEGNVFSCSVMHEALHNHYTQKSLSLSLGK

SEQ ID NO: 8 - protein, 208 aa, Homo sapiens
EIVLTQSPATLSLSPGERATLSCRASQSVSSYLAWYQQPGQAPRLLIYDASNRATGIPARFSGSGSGTDFTLTISSLEPEDFAVYYCQQSSNWPRTFGQGTKVEIRTVAAPSVFIFPPSDEQLSGTASVVCLLNNFYPREAVQWKVDNALQSGNSQESVTEQDSDSTYSLSSTLTLSKADYEKHKVYACEVTHQGLSSPVTSFNRGEC

SEQ ID NO: 9 - protein, 447 aa, Artificial Sequence
  Synthetic Construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 218 aa, Artificial Sequence
  Synthetic Construct
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 432 aa, Artificial Sequence
  Synthetic Construct
LFTVTVPKELYIIEHGSNVTLECNFDTGSHVNLGAITASLQKVENDTSPHRERATLLEEQLPLGKASFHIPQVQVRDEGQYQCIIIYGVAWDYKYLTLKVKASYRKINTHILKVPETDEVELTCQATGYPLAEVSWPNVSVPANTSHSRTPEGLYQVTSVLRLKPPPGRNFSCVFWNTHVRELTLASIDLQSQMEPRTHPTWEPKSCDKTHTCPPCPAPELLGGPSVFLFPPKPKDTLMISRTPEVTCWVDVSHEDPEVKFNWYVDGVEVHNAKTKPREEQYNSTYRWSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 118 aa, Artificial Sequence
  Synthetic Construct
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSA

SEQ ID NO: 13 - protein, 108 aa, Artificial Sequence
  Synthetic Construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKR

SEQ ID NO: 14 - protein, 117 aa, Artificial Sequence
  Synthetic Construct
EVQLLESGGGLVQPGGSLRLSCAASGFTFSSYIMWVRQAPGKGLEWVSSIYPSGGITFYADKGRFTISRDNSKNTLYLQMNSLRAEDTAVYYCARIKLGTVTTVDYWGQGTLVTVSS

SEQ ID NO: 15 - protein, 110 aa, Artificial Sequence
  Synthetic Construct
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVL

SEQ ID NO: 16 - protein, 290 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 17 - protein, 5 aa, Artificial Sequence
  Synthetic Construct
SYIMM

SEQ ID NO: 18 - protein, 11 aa, Artificial Sequence
  Synthetic Construct
SIYPSGGITFY

SEQ ID NO: 19 - protein, 11 aa, Artificial Sequence
  Synthetic Construct
IKLGTVTTVDY

SEQ ID NO: 20 - protein, 14 aa, Artificial Sequence
  Synthetic Construct
TGTSSDVGGYNYVS

SEQ ID NO: 21 - protein, 7 aa, Artificial Sequence
  Synthetic Construct
DVSNRPS

SEQ ID NO: 22 - protein, 10 aa, Artificial Sequence
  Synthetic Construct
SSYTSSSTRV

SEQ ID NO: 23 - protein, 450 aa, Artificial Sequence
  Synthetic Construct
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 216 aa, Artificial Sequence
  Synthetic Construct
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS